(19) United States
(12) Patent Application Publication
(10) Pub. No.: US 2021/0403579 A1
(43) Pub. Date: Dec. 30, 2021

(54) MULTIVALENT REGULATORY T CELL MODULATORS

(71) Applicant: Delinia, Inc., Emeryville, CA (US)

(72) Inventors: Jungmin Kim, Berkeley, CA (US); Niranjana Nagarajan, Oakland, CA (US); Jeffrey Greve, Berkeiey, CA (US)

(73) Assignee: Delinia, Inc., Emeryville, CA (US)

(21) Appl. No.: 17/290,129

(22) PCT Filed: Oct. 30, 2019
(86) PCT No.: PCT/US2019/058854

§ 371 Date: Apr. 29, 2021

Related U.S. Application Data
(60) Provisional application No. 62/753,397, filed on Oct. 31, 2018.

(51) Int. Cl. C07K 16/28; C07K 14/55; A61P 1/00

(52) U.S. Cl. CPC C07K 16/2866; C07K 14/55; A61P 1/00; A61K 2039/505; C07K 2319/30; C07K 2317/21; C07K 2317/31

(57) ABSTRACT

This disclosure provides compounds that contain an IL-2 receptor-binding domain and an ST2-binding domain, e.g. an antibody or fragment thereof that specifically binds ST2. The methods described in the present disclosure provide for a method for treating a condition by administering to a subject in need thereof a therapeutically-effective amount of a compound containing an IL-2 receptor-binding domain and an ST2-binding domain.

### RELATED APPLICATIONS

This application claims priority to U.S. Provisional Patent Application No. 62/753,397 filed on Oct. 31, 2018, the contents of which are incorporated herein in their entirety.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is 127754_00820_Sequence_Listing. The size of the text file is 1158 KB, and the text file was created on Oct. 30, 2019.

### BACKGROUND OF THE INVENTION

Inflammatory myopathies are conditions that are characterized by chronic muscle inflammation and muscle weakness. Muscular dystrophies are degenerative muscle diseases caused by a mutated dystrophin gene, but an underlying cause of the progressive degeneration is muscle inflammation. The inflammation associated with these diseases can damage muscle fibers, causing fatigue, pain, and progressive muscle degeneration. Regulatory T cells (Tregs) are a specialized subset of T cells. Tregs suppress activation of the immune system and thereby regulate the self-tolerance of the immune system. Subsets of Tregs expressing defined molecular markers, such as the receptor ST2, are found in inflamed tissues, such as injured skeletal muscle and inflamed lungs. Expansion and activation of ST2-expressing Tregs have been implicated in the resolution of acute muscle injury and of muscle inflammation associated with muscular dystrophy. Additionally, ST2+ Tregs are found in tissues such as visceral adipose, colon, and lung, and possess immunoregulatory and tissue repair functions in those tissues.

### SUMMARY OF THE INVENTION

In one aspect, the disclosure relates to a fusion protein comprising: a) a human IL-2 protein domain; b) an immunoglobulin Fc protein domain; and c) a protein domain that binds to Interleukin 1 receptor-like 1 (ST2). In certain embodiments, the protein domain that binds to ST2 is an antibody specific for ST2, or an antigen-binding fragment thereof. In certain embodiments, the fusion protein further comprises at least one peptide linker domain. In certain embodiments, the human IL-2 protein domain comprises human IL-2 with a substitution selected from the group consisting of: T3A, N88R, N88G, D20H, C125S, Q126L, and Q126F, relative to the amino acid sequence of SEQ ID NO: 2. In certain embodiments, the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9. In certain embodiments, the fusion protein comprises a human IgG1 heavy chain listed in Table 1, or a fragment thereof. In certain embodiments, the antibody specific for ST2, or the antigen-binding fragment thereof, comprises an IgG1 heavy chain fragment listed in Table 2. In certain embodiments, the peptide linker domain comprises the amino acid sequence of SEQ ID NO: 6. In certain embodiments, the fusion protein comprises a first peptide linker domain and a second peptide linker domain.

In certain embodiments of the fusion proteins described herein, each domain has an amino-terminus (N-terminus) and a carboxy terminus (C-terminus); wherein the fusion protein is configured so that a) the C-terminus of the human IL-2 protein domain is fused through a peptide bond to the N-terminus of the first peptide linker domain; b) the N-terminus of the IgG Fc protein domain is fused through a peptide bond to the C-terminus of the first peptide linker domain; c) the N-terminus of the second peptide linker domain is fused through a peptide bond to the C-terminus of the IgG Fc protein domain; and d) the N-terminus of the protein domain that binds to ST2 is fused through a peptide bond to the C-terminus of the second peptide linker domain. In certain embodiments, the fusion protein comprises the amino acid sequence of SEQ ID NO: 12, SEQ ID NO: 13, SEQ ID NO: 14, or SEQ ID NO: 15.

In one aspect, the disclosure relates to a nucleic acid encoding a fusion protein described herein.

In one aspect, the disclosure relates to a dimeric protein comprising a fusion protein described herein. In certain embodiments, the dimeric protein comprises a first fusion protein and a second fusion protein, wherein: each fusion protein comprises an immunoglobulin (IgG) Fc protein domain and at least one additional protein domain selected from the group consisting of i. a human IL-2 protein domain; and ii. a protein domain that binds to Interleukin 1 receptor-like 1 (ST2); and b. the dimeric protein comprises at least one human IL-2 protein domain and at least one protein domain that binds to ST2. In certain embodiments, the first fusion protein comprises a human IL-2 protein domain, a first immunoglobulin Fc protein domain, and a first peptide linker; and the second fusion protein comprises a protein domain that binds to ST2, a second immunoglobulin Fc protein domain, and a second peptide linker domain. In certain embodiments, a. each domain has an amino-terminus (N-terminus) and a carboxy terminus (C-terminus); b. the first fusion protein is configured so that i. the C-terminus of the human IL-2 protein domain is fused through a peptide bond to the N-terminus of the first peptide linker domain; and ii. the N-terminus of the first IgG Fc protein domain is fused through a peptide bond to the C-terminus of the first peptide linker domain; and c. the second fusion protein is configured so that i. the C-terminus of the second IgG Fc protein domain is fused through a peptide bond to the N-terminus of the second peptide linker domain; and ii. the N-terminus of the protein domain that binds to ST2 is fused through a peptide bond to the C-terminus of the second peptide linker domain. In certain embodiments, the protein domain that binds to ST2 is an antibody specific for ST2, or an antigen-binding fragment thereof. In certain embodiments, at least one of the fusion proteins comprises a human IgG1 ST2 antibody listed in Table 1. In certain embodiments, the antibody specific for ST2, or the antigen-binding fragment thereof, comprises an IgG1 heavy chain fragment listed in Table 2. In certain embodiments, the antibody specific for ST2, or the antigen-binding fragment thereof, comprises a light chain amino acid sequence listed in Table 1. In certain embodiments, at least one of the fusion proteins further comprises at least one peptide linker domain. In certain embodiments, the human IL-2 protein domain comprises human IL-2 with a substitution selected from the group consisting of: T3A, N88R, N88G, D20H, C125S, Q126L, and Q126F, relative to the amino acid sequence of SEQ ID NO: 2. In certain embodiments, the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9. In certain embodiments, the peptide linker domain comprises the amino acid sequence of SEQ ID NO: 6.

In certain embodiments, a. the first fusion protein and the second fusion protein each comprise the amino acid sequence of SEQ ID NO: 12, and the dimeric protein further comprises the amino acid sequence of SEQ ID NO: 19; b. the first fusion protein and the second fusion protein each comprise the amino acid sequence of SEQ ID NO: 13, and the dimeric protein further comprise the amino acid sequence of SEQ ID NO: 20; c. the first fusion protein comprises the amino acid sequence of SEQ ID NO: 16, the second fusion protein comprises the amino acid sequence of SEQ ID NO: 14, and the dimeric protein further comprises the amino acid sequence of SEQ ID NO: 19; d. the first fusion protein comprises the amino acid sequence of SEQ ID NO: 17, the second fusion protein comprises the amino acid sequence of SEQ ID NO: 15, and the dimeric protein further comprises the amino acid sequence of SEQ ID NO: 20; e. the first fusion protein comprises the amino acid sequence of SEQ ID NO: 16, the second fusion protein comprises the amino acid sequence of SEQ ID NO: 11, and the dimeric protein further comprises the amino acid sequence of SEQ ID NO: 19; f. the first fusion protein comprises the amino acid sequence of SEQ ID NO: 17, the second fusion protein comprises the amino acid sequence of SEQ ID NO: 11, and the dimeric protein further comprises the amino acid sequence of SEQ ID NO: 20; g. the first fusion protein comprises the amino acid sequence of SEQ ID NO: 16, the second fusion protein comprises the amino acid sequence of SEQ ID NO: 10, and the dimeric protein further comprises the amino acid sequence of SEQ ID NO: 19; h. the first fusion protein comprises the amino acid sequence of SEQ ID NO: 17, the second fusion protein comprises the amino acid sequence of SEQ ID NO: 10, and the dimeric protein further comprises the amino acid sequence of SEQ ID NO: 20; i. the first fusion protein comprises the amino acid sequence of SEQ ID NO: 18, the second fusion protein comprises the amino acid sequence of SEQ ID NO: 14, and the dimeric protein further comprises the amino acid sequence of SEQ ID NO: 19; or j. the first fusion protein comprises the amino acid sequence of SEQ ID NO: 18, the second fusion protein comprises the amino acid sequence of SEQ ID NO: 15, and the dimeric protein further comprises the amino acid sequence of SEQ ID NO: 20.

In one aspect, the disclosure relates to a pharmaceutical composition comprising a dimeric protein as disclosed herein. In one aspect, the disclosure relates to a method for treating a condition, the method comprising administering to a subject in need thereof a therapeutically-effective amount of the pharmaceutical composition as disclosed herein. In certain embodiments, the condition is selected from the group consisting of an inflammatory myopathy, muscular dystrophy, polymyositis, dermatomyositis, an inflammatory condition of adipose tissue, an inflammatory condition of the colon, an inflammatory condition of the lung, Graft-vs-Host Disease, Pemphigus Vulgaris, Systemic Lupus Erythematosus, Scleroderma, Ulcerative Colitis, Crohn's Disease, Psoriasis, Type 1 Diabetes, Multiple Sclerosis, Amyotrophic Lateral Sclerosis, Alopecia Areata, Uveitis, Neuromyelitis Optica, and Duchenne Muscular Dystrophy. In certain embodiments, the administration is subcutaneous.

In one aspect, the disclosure relates to a method of selectively activating an ST2⁺ regulatory T cell relative to an ST2⁻ regulatory T cell in a subject, the method comprising administering to the subject a therapeutically effective amount of a pharmaceutical composition as disclosed herein.

In certain aspects the disclosure relates to a recombinant antibody or an antigen-binding fragment thereof that specifically binds ST2, comprising:

(i) a light chain variable region having at least 85%, 90%, 95%, 96%, 97%, 98% or 99% sequence identity to an amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457; and
(ii) a heavy chain variable region having at least 90%, 95%, 96%, 97%, 98%, or 99% sequence identity to an amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239.

In certain embodiments, the antibody or antigen-binding fragment comprises:

a light chain variable region comprising amino acids 21-132 of SEQ ID NO: 241 and a heavy chain variable region comprising amino acids 25-147 of SEQ ID NO: 23;
a light chain variable region comprising amino acids 21-132 of SEQ ID NO: 243 and a heavy chain variable region comprising amino acids 25-147 of SEQ ID NO: 25;
a light chain variable region comprising amino acids 21-128 of SEQ ID NO: 245 and a heavy chain variable region comprising amino acids 25-145 of SEQ ID NO: 27;
a light chain variable region comprising amino acids 21-127 of SEQ ID NO: 247 and a heavy chain variable region comprising amino acids 25-148 of SEQ ID NO: 29;
a light chain variable region comprising amino acids 21-127 of SEQ ID NO: 249 and a heavy chain variable region comprising amino acids 25-141 of SEQ ID NO: 31;
a light chain variable region comprising amino acids 21-127 of SEQ ID NO: 251 and a heavy chain variable region comprising amino acids 25-143 of SEQ ID NO: 33;
a light chain variable region comprising amino acids 21-131 of SEQ ID NO: 253 and a heavy chain variable region comprising amino acids 25-150 of SEQ ID NO: 35;
a light chain variable region comprising amino acids 21-132 of SEQ ID NO: 255 and a heavy chain variable region comprising amino acids 25-148 of SEQ ID NO: 37;
a light chain variable region comprising amino acids 21-127 of SEQ ID NO: 257 and a heavy chain variable region comprising amino acids 25-146 of SEQ ID NO: 39;
a light chain variable region comprising amino acids 21-132 of SEQ ID NO: 259 and a heavy chain variable region comprising amino acids 25-145 of SEQ ID NO: 41;
a light chain variable region comprising amino acids 21-127 of SEQ ID NO: 261 and a heavy chain variable region comprising amino acids 25-143 of SEQ ID NO: 43;
a light chain variable region comprising amino acids 21-127 of SEQ ID NO: 263 and a heavy chain variable region comprising amino acids 25-151 of SEQ ID NO: 45;
a light chain variable region comprising amino acids 21-132 of SEQ ID NO: 265 and a heavy chain variable region comprising amino acids 25-141 of SEQ ID NO: 47;
a light chain variable region comprising amino acids 21-132 of SEQ ID NO: 267 and a heavy chain variable region comprising amino acids 25-140 of SEQ ID NO: 49;
a light chain variable region comprising amino acids 21-127 of SEQ ID NO: 269 and a heavy chain variable region comprising amino acids 25-145 of SEQ ID NO: 51;
a light chain variable region comprising amino acids 1-127 of SEQ ID NO: 271 and a heavy chain variable region comprising amino acids 25-152 of SEQ ID NO: 53;
a light chain variable region comprising amino acids 21-127 of SEQ ID NO: 273 and a heavy chain variable region comprising amino acids 25-142 of SEQ ID NO: 55;
a light chain variable region comprising amino acids 21-127 of SEQ ID NO: 275 and a heavy chain variable region comprising amino acids 25-147 of SEQ ID NO: 57;
a light chain variable region comprising amino acids 21-127 of SEQ ID NO: 277 and a heavy chain variable region comprising amino acids 25-141 of SEQ ID NO: 59;
a light chain variable region comprising amino acids 21-127 of SEQ ID NO: 279 and a heavy chain variable region comprising amino acids 25-148 of SEQ ID NO: 61;
a light chain variable region comprising amino acids 21-127 of SEQ ID NO: 281 and a heavy chain variable region comprising amino acids 25-142 of SEQ ID NO: 63;
a light chain variable region comprising amino acids 21-127 of SEQ ID NO: 283 and a heavy chain variable region comprising amino acids 25-145 of SEQ ID NO: 65;
a light chain variable region comprising amino acids 21-127 of SEQ ID NO: 285 and a heavy chain variable region comprising amino acids 25-140 of SEQ ID NO: 67;
a light chain variable region comprising amino acids 21-132 of SEQ ID NO: 287 and a heavy chain variable region comprising amino acids 25-145 of SEQ ID NO: 69;
a light chain variable region comprising amino acids 21-127 of SEQ ID NO: 289 and a heavy chain variable region comprising amino acids 25-140 of SEQ ID NO: 71;
a light chain variable region comprising amino acids 21-133 of SEQ ID NO: 291 and a heavy chain variable region comprising amino acids 25-140 of SEQ ID NO: 73;
a light chain variable region comprising amino acids 21-127 of SEQ ID NO: 293 and a heavy chain variable region comprising amino acids 25-145 of SEQ ID NO: 75;
a light chain variable region comprising amino acids 21-132 of SEQ ID NO: 295 and a heavy chain variable region comprising amino acids 25-143 of SEQ ID NO: 77;
a light chain variable region comprising amino acids 21-127 of SEQ ID NO: 297 and a heavy chain variable region comprising amino acids 25-143 of SEQ ID NO: 79;
a light chain variable region comprising amino acids 21-132 of SEQ ID NO: 299 and a heavy chain variable region comprising amino acids 25-151 of SEQ ID NO: 81;
a light chain variable region comprising amino acids 21-127 of SEQ ID NO: 301 and a heavy chain variable region comprising amino acids 25-142 of SEQ ID NO: 83;
a light chain variable region comprising amino acids 21-127 of SEQ ID NO: 303 and a heavy chain variable region comprising amino acids 25-144 of SEQ ID NO: 85;
a light chain variable region comprising amino acids 21-132 of SEQ ID NO: 305 and a heavy chain variable region comprising amino acids 25-148 of SEQ ID NO: 87;
a light chain variable region comprising amino acids 21-127 of SEQ ID NO: 307 and a heavy chain variable region comprising amino acids 25-144 of SEQ ID NO: 89;
a light chain variable region comprising amino acids 21-127 of SEQ ID NO: 309 and a heavy chain variable region comprising amino acids 25-140 of SEQ ID NO: 91;
a light chain variable region comprising amino acids 21-127 of SEQ ID NO: 311 and a heavy chain variable region comprising amino acids 25-143 of SEQ ID NO: 93;
a light chain variable region comprising amino acids 21-127 of SEQ ID NO: 313 and a heavy chain variable region comprising amino acids 25-150 of SEQ ID NO: 95;
a light chain variable region comprising amino acids 21-128 of SEQ ID NO: 315 and a heavy chain variable region comprising amino acids 25-147 of SEQ ID NO: 97;
a light chain variable region comprising amino acids 21-132 of SEQ ID NO: 317 and a heavy chain variable region comprising amino acids 25-141 of SEQ ID NO: 99;
a light chain variable region comprising amino acids 21-127 of SEQ ID NO: 319 and a heavy chain variable region comprising amino acids 25-153 of SEQ ID NO: 101;
a light chain variable region comprising amino acids 21-133 of SEQ ID NO: 321 and a heavy chain variable region comprising amino acids 25-152 of SEQ ID NO: 103;
a light chain variable region comprising amino acids 21-127 of SEQ ID NO: 323 and a heavy chain variable region comprising amino acids 25-145 of SEQ ID NO: 105;
a light chain variable region comprising amino acids 21-127 of SEQ ID NO: 325 and a heavy chain variable region comprising amino acids 25-144 of SEQ ID NO: 107;
a light chain variable region comprising amino acids 21-127 of SEQ ID NO: 327 and a heavy chain variable region comprising amino acids 25-146 of SEQ ID NO: 109;
a light chain variable region comprising amino acids 21-127 of SEQ ID NO: 329 and a heavy chain variable region comprising amino acids 25-140 of SEQ ID NO: 111;
a light chain variable region comprising amino acids 21-127 of SEQ ID NO: 331 and a heavy chain variable region comprising amino acids 25-143 of SEQ ID NO: 113;
a light chain variable region comprising amino acids 21-127 of SEQ ID NO: 333 and a heavy chain variable region comprising amino acids 25-144 of SEQ ID NO: 115;
a light chain variable region comprising amino acids 21-127 of SEQ ID NO: 335 and a heavy chain variable region comprising amino acids 25-141 of SEQ ID NO: 117;
a light chain variable region comprising amino acids 21-127 of SEQ ID NO: 337 and a heavy chain variable region comprising amino acids 25-149 of SEQ ID NO: 119;
a light chain variable region comprising amino acids 21-127 of SEQ ID NO: 339 and a heavy chain variable region comprising amino acids 25-145 of SEQ ID NO: 121;
a light chain variable region comprising amino acids 21-128 of SEQ ID NO: 341 and a heavy chain variable region comprising amino acids 25-149 of SEQ ID NO: 123;
a light chain variable region comprising amino acids 21-131 of SEQ ID NO: 343 and a heavy chain variable region comprising amino acids 25-149 of SEQ ID NO: 125;
a light chain variable region comprising amino acids 21-127 of SEQ ID NO: 345 and a heavy chain variable region comprising amino acids 25-147 of SEQ ID NO: 127;
a light chain variable region comprising amino acids 21-131 of SEQ ID NO: 347 and a heavy chain variable region comprising amino acids 25-147 of SEQ ID NO: 129;
a light chain variable region comprising amino acids 21-132 of SEQ ID NO: 349 and a heavy chain variable region comprising amino acids 25-145 of SEQ ID NO: 131;
a light chain variable region comprising amino acids 21-127 of SEQ ID NO: 351 and a heavy chain variable region comprising amino acids 25-146 of SEQ ID NO: 133;
a light chain variable region comprising amino acids 21-127 of SEQ ID NO: 353 and a heavy chain variable region comprising amino acids 25-152 of SEQ ID NO: 135;
a light chain variable region comprising amino acids 21-127 of SEQ ID NO: 355 and a heavy chain variable region comprising amino acids 25-146 of SEQ ID NO: 137;
a light chain variable region comprising amino acids 21-127 of SEQ ID NO: 357 and a heavy chain variable region comprising amino acids 25-149 of SEQ ID NO: 139;
a light chain variable region comprising amino acids 21-127 of SEQ ID NO: 359 and a heavy chain variable region comprising amino acids 25-149 of SEQ ID NO: 141;
a light chain variable region comprising amino acids 21-132 of SEQ ID NO: 361 and a heavy chain variable region comprising amino acids 25-145 of SEQ ID NO: 143;
a light chain variable region comprising amino acids 21-133 of SEQ ID NO: 363 and a heavy chain variable region comprising amino acids 25-142 of SEQ ID NO: 145;
a light chain variable region comprising amino acids 21-132 of SEQ ID NO: 365 and a heavy chain variable region comprising amino acids 25-147 of SEQ ID NO: 147;
a light chain variable region comprising amino acids 21-126 of SEQ ID NO: 367 and a heavy chain variable region comprising amino acids 25-141 of SEQ ID NO: 149;
a light chain variable region comprising amino acids 21-127 of SEQ ID NO: 369 and a heavy chain variable region comprising amino acids 25-140 of SEQ ID NO: 151;
a light chain variable region comprising amino acids 21-132 of SEQ ID NO: 371 and a heavy chain variable region comprising amino acids 25-145 of SEQ ID NO: 153;
a light chain variable region comprising amino acids 21-127 of SEQ ID NO: 373 and a heavy chain variable region comprising amino acids 25-153 of SEQ ID NO: 155;
a light chain variable region comprising amino acids 21-132 of SEQ ID NO: 375 and a heavy chain variable region comprising amino acids 25-146 of SEQ ID NO: 157;
a light chain variable region comprising amino acids 21-132 of SEQ ID NO: 377 and a heavy chain variable region comprising amino acids 25-149 of SEQ ID NO: 159;
a light chain variable region comprising amino acids 21-128 of SEQ ID NO: 379 and a heavy chain variable region comprising amino acids 25-141 of SEQ ID NO: 161;
a light chain variable region comprising amino acids 21-127 of SEQ ID NO: 381 and a heavy chain variable region comprising amino acids 25-156 of SEQ ID NO: 163;
a light chain variable region comprising amino acids 21-127 of SEQ ID NO: 383 and a heavy chain variable region comprising amino acids 25-141 of SEQ ID NO: 165;
a light chain variable region comprising amino acids 21-127 of SEQ ID NO: 385 and a heavy chain variable region comprising amino acids 25-140 of SEQ ID NO: 167;
a light chain variable region comprising amino acids 21-127 of SEQ ID NO: 387 and a heavy chain variable region comprising amino acids 25-140 of SEQ ID NO: 169;
a light chain variable region comprising amino acids 21-127 of SEQ ID NO: 389 and a heavy chain variable region comprising amino acids 25-141 of SEQ ID NO: 171;
a light chain variable region comprising amino acids 21-127 of SEQ ID NO: 391 and a heavy chain variable region comprising amino acids 25-140 of SEQ ID NO: 173;
a light chain variable region comprising amino acids 21-133 of SEQ ID NO: 393 and a heavy chain variable region comprising amino acids 25-144 of SEQ ID NO: 175;
a light chain variable region comprising amino acids 21-127 of SEQ ID NO: 395 and a heavy chain variable region comprising amino acids 25-142 of SEQ ID NO: 177;
a light chain variable region comprising amino acids 21-127 of SEQ ID NO: 397 and a heavy chain variable region comprising amino acids 25-145 of SEQ ID NO: 179;
a light chain variable region comprising amino acids 21-132 of SEQ ID NO: 399 and a heavy chain variable region comprising amino acids 25-145 of SEQ ID NO: 181;
a light chain variable region comprising amino acids 21-127 of SEQ ID NO: 401 and a heavy chain variable region comprising amino acids 25-143 of SEQ ID NO: 183;
a light chain variable region comprising amino acids 21-127 of SEQ ID NO: 403 and a heavy chain variable region comprising amino acids 25-147 of SEQ ID NO: 185;
a light chain variable region comprising amino acids 21-127 of SEQ ID NO: 405 and a heavy chain variable region comprising amino acids 25-143 of SEQ ID NO: 187;
a light chain variable region comprising amino acids 21-132 of SEQ ID NO: 407 and a heavy chain variable region comprising amino acids 25-145 of SEQ ID NO: 189;
a light chain variable region comprising amino acids 21-127 of SEQ ID NO: 409 and a heavy chain variable region comprising amino acids 25-144 of SEQ ID NO: 191;
a light chain variable region comprising amino acids 21-127 of SEQ ID NO: 411 and a heavy chain variable region comprising amino acids 25-143 of SEQ ID NO: 193;
a light chain variable region comprising amino acids 21-127 of SEQ ID NO: 413 and a heavy chain variable region comprising amino acids 25-146 of SEQ ID NO: 195;
a light chain variable region comprising amino acids 21-127 of SEQ ID NO: 415 and a heavy chain variable region comprising amino acids 25-141 of SEQ ID NO: 197;
a light chain variable region comprising amino acids 21-127 of SEQ ID NO: 417 and a heavy chain variable region comprising amino acids 25-146 of SEQ ID NO: 199;
a light chain variable region comprising amino acids 21-132 of SEQ ID NO: 419 and a heavy chain variable region comprising amino acids 25-142 of SEQ ID NO: 201;
a light chain variable region comprising amino acids 21-127 of SEQ ID NO: 421 and a heavy chain variable region comprising amino acids 25-139 of SEQ ID NO: 203;
a light chain variable region comprising amino acids 21-133 of SEQ ID NO: 423 and a heavy chain variable region comprising amino acids 25-144 of SEQ ID NO: 205;
a light chain variable region comprising amino acids 21-132 of SEQ ID NO: 425 and a heavy chain variable region comprising amino acids 25-146 of SEQ ID NO: 207;
a light chain variable region comprising amino acids 21-128 of SEQ ID NO: 427 and a heavy chain variable region comprising amino acids 25-142 of SEQ ID NO: 209;
a light chain variable region comprising amino acids 21-132 of SEQ ID NO: 429 and a heavy chain variable region comprising amino acids 25-151 of SEQ ID NO: 211;
a light chain variable region comprising amino acids 21-132 of SEQ ID NO: 431 and a heavy chain variable region comprising amino acids 25-141 of SEQ ID NO: 213;
a light chain variable region comprising amino acids 21-127 of SEQ ID NO: 433 and a heavy chain variable region comprising amino acids 25-140 of SEQ ID NO: 215;
a light chain variable region comprising amino acids 21-127 of SEQ ID NO: 435 and a heavy chain variable region comprising amino acids 25-146 of SEQ ID NO: 217;
a light chain variable region comprising amino acids 21-127 of SEQ ID NO: 437 and a heavy chain variable region comprising amino acids 25-142 of SEQ ID NO: 219;
a light chain variable region comprising amino acids 21-127 of SEQ ID NO: 439 and a heavy chain variable region comprising amino acids 25-143 of SEQ ID NO: 221;
a light chain variable region comprising amino acids 21-126 of SEQ ID NO: 441 and a heavy chain variable region comprising amino acids 25-150 of SEQ ID NO: 223;
a light chain variable region comprising amino acids 21-132 of SEQ ID NO: 443 and a heavy chain variable region comprising amino acids 25-144 of SEQ ID NO: 225;
a light chain variable region comprising amino acids 21-127 of SEQ ID NO: 445 and a heavy chain variable region comprising amino acids 25-145 of SEQ ID NO: 227;
a light chain variable region comprising amino acids 21-127 of SEQ ID NO: 447 and a heavy chain variable region comprising amino acids 25-149 of SEQ ID NO: 229;
a light chain variable region comprising amino acids 21-127 of SEQ ID NO: 449 and a heavy chain variable region comprising amino acids 251-147 of SEQ ID NO: 231;
a light chain variable region comprising amino acids 21-128 of SEQ ID NO: 451 and a heavy chain variable region comprising amino acids 25-140 of SEQ ID NO: 233;
a light chain variable region comprising amino acids 21-127 of SEQ ID NO: 453 and a heavy chain variable region comprising amino acids 25-141 of SEQ ID NO: 235;
a light chain variable region comprising amino acids 21-127 of SEQ ID NO: 455 and a heavy chain variable region comprising amino acids 25-140 of SEQ ID NO: 237; or
a light chain variable region comprising amino acids 21-133 of SEQ ID NO: 457 and a heavy chain variable region comprising amino acids 25-150 of SEQ ID NO: 239.

In certain embodiments, the antibody or antigen-binding fragment comprises a heavy chain fragment selected from the group consisting of:

amino acids 25-251 of SEQ ID NO: 23, amino acids 25-250 of SEQ ID NO: 25, amino acids 25-249 of SEQ ID NO: 27, amino acids 25-252 of SEQ ID NO: 29, amino acids 25-245 of SEQ ID NO: 31, amino acids 25-247 of SEQ ID NO: 33, amino acids 25-254 of SEQ ID NO: 35, amino acids 25-252 of SEQ ID NO: 37, amino acids 25-250 of SEQ ID NO: 39, amino acids 25-249 of SEQ ID NO: 41, amino acids 25-248 of SEQ ID NO: 43, amino acids 25-255 of SEQ ID NO: 45, amino acids 25-245 of SEQ ID NO: 47, amino acids 25-244 of SEQ ID NO: 49, amino acids 25-249 of SEQ ID NO: 51, amino acids 25-256 of SEQ ID NO: 53, amino acids 25-246 of SEQ ID NO: 55, amino acids 25-251 of SEQ ID NO: 57, amino acids 25-245 of SEQ ID NO: 59, amino acids 25-252 of SEQ ID NO: 61, amino acids 25-246 of SEQ ID NO: 63, amino acids 25-249 of SEQ ID NO: 65, amino acids 25-244 of SEQ ID NO: 67, amino acids 25-249 of SEQ ID NO: 69, amino acids 25-244 of SEQ ID NO: 71, amino acids 25-244 of SEQ ID NO: 73, amino acids 25-249 of SEQ ID NO: 75, amino acids 25-247 of SEQ ID NO: 77, amino acids 25-247 of SEQ ID NO: 79, amino acids 25-255 of SEQ ID NO: 81, amino acids 25-246 of SEQ ID NO: 83, amino acids 25-248 of SEQ ID NO: 85, amino acids 25-252 of SEQ ID NO: 87, amino acids 25-248 of SEQ ID NO: 89, amino acids 25-244 of SEQ ID NO: 91, amino acids 25-247 of SEQ ID NO: 93, amino acids 25-254 of SEQ ID NO: 95, amino acids 25-251 of SEQ ID NO: 97, amino acids 25-245 of SEQ ID NO: 99, amino acids 25-257 of SEQ ID NO: 101, amino acids 25-256 of SEQ ID NO: 103, amino acids 25-249 of SEQ ID NO: 105, amino acids 25-248 of SEQ ID NO: 107, amino acids 25-250 of SEQ ID NO: 109, amino acids 25-244 of SEQ ID NO: 111, amino acids 25-247 of SEQ ID NO: 113, amino acids 25-248 of SEQ ID NO: 115, amino acids 25-245 of SEQ ID NO: 117, amino acids 25-253 of SEQ ID NO: 119, amino acids 25-249 of SEQ ID NO: 121, amino acids 25-253 of SEQ ID NO: 123, amino acids 25-253 of SEQ ID NO: 125, amino acids 25-251 of SEQ ID NO: 127, amino acids 25-245 of SEQ ID NO: 129, amino acids 25-249 of SEQ ID NO: 131, amino acids 25-250 of SEQ ID NO: 133, amino acids 25-256 of SEQ ID NO: 135, amino acids 25-250 of SEQ ID NO: 137, amino acids 25-247 of SEQ ID NO: 139, amino acids 25-253 of SEQ ID NO: 141, amino acids 25-249 of SEQ ID NO: 143, amino acids 25-246 of SEQ ID NO: 145, amino acids 25-251 of SEQ ID NO: 147, amino acids 25-245 of SEQ ID NO: 149, amino acids 25-244 of SEQ ID NO: 151, amino acids 25-249 of SEQ ID NO: 153, amino acids 25-257 of SEQ ID NO: 155, amino acids 25-250 of SEQ ID NO: 157, amino acids 25-253 of SEQ ID NO: 159, amino acids 25-245 of SEQ ID NO: 161, amino acids 25-260 of SEQ ID NO: 163, amino acids 25-245 of SEQ ID NO: 165, amino acids 25-244 of SEQ ID NO: 167, amino acids 25-244 of SEQ ID NO: 169, amino acids 25-245 of SEQ ID NO: 171, amino acids 25-244 of SEQ ID NO: 173, amino acids 25-248 of SEQ ID NO: 175, amino acids 25-246 of SEQ ID NO: 177, amino acids 1-249 of SEQ ID NO: 179, amino acids 1-249 of SEQ ID NO: 181, amino acids 1-247 of SEQ ID NO: 183, amino acids 1-251 of SEQ ID NO: 185, amino acids 1-247 of SEQ ID NO: 187, amino acids 25-249 of SEQ ID NO: 189, amino acids 25-248 of SEQ ID NO: 191, amino acids 25-246 of SEQ ID NO: 193, amino acids 25-250 of SEQ ID NO: 195, amino acids 25-245 of SEQ ID NO: 197, amino acids 25-250 of SEQ ID NO: 199, amino acids 25-246 of SEQ ID NO: 201, amino acids 25-243 of SEQ ID NO: 203, amino acids 25-248 of SEQ ID NO: 205, amino acids 25-250 of SEQ ID NO: 207, amino acids 25-249 of SEQ ID NO: 209, amino acids 25-255 of SEQ ID NO: 211, amino acids 25-245 of SEQ ID NO: 213, amino acids 25-244 of SEQ ID NO: 215, amino acids 25-250 of SEQ ID NO: 217, amino acids 25-249 of SEQ ID NO: 219, amino acids 25-247 of SEQ ID NO: 221, amino acids 25-254 of SEQ ID NO: 223, amino acids 25-248 of SEQ ID NO: 225, amino acids 25-249 of SEQ ID NO: 227, amino acids 25-253 of SEQ ID NO: 229, amino acids 25-251 of SEQ ID NO: 231, amino acids 25-244 of SEQ ID NO: 233, amino acids 25-245 of SEQ ID NO: 235, amino acids 25-244 of SEQ ID NO: 237, and amino acids 25-254 of SEQ ID NO: 239.

In certain embodiments, the antibody or antigen-binding fragment comprises:

a heavy chain fragment comprising amino acids 25-251 of SEQ ID NO: 23 and a light chain comprising SEQ ID NO: 241;
a heavy chain fragment comprising amino acids 25-250 of SEQ ID NO: 25 and a light chain comprising SEQ ID NO: 243;
a heavy chain fragment comprising amino acids 25-249 of SEQ ID NO: 27 and a light chain comprising SEQ ID NO: 245;
a heavy chain fragment comprising amino acids 25-252 of SEQ ID NO: 29 and a light chain comprising SEQ ID NO: 247;
a heavy chain fragment comprising amino acids 25-245 of SEQ ID NO: 31 and a light chain comprising SEQ ID NO: 249;
a heavy chain fragment comprising amino acids 25-247 of SEQ ID NO: 33 and a light chain comprising SEQ ID NO: 251;
a heavy chain fragment comprising amino acids 25-254 of SEQ ID NO: 35 and a light chain comprising SEQ ID NO: 253;
a heavy chain fragment comprising amino acids 25-252 of SEQ ID NO: 37 and a light chain comprising SEQ ID NO: 255;
a heavy chain fragment comprising amino acids 25-250 of SEQ ID NO: 39 and a light chain comprising SEQ ID NO: 257;
a heavy chain fragment comprising amino acids 25-249 of SEQ ID NO: 41 and a light chain comprising SEQ ID NO: 259;
a heavy chain fragment comprising amino acids 25-248 of SEQ ID NO: 43 and a light chain comprising SEQ ID NO: 261;
a heavy chain fragment comprising amino acids 25-255 of SEQ ID NO: 45 and a light chain comprising SEQ ID NO: 263;
a heavy chain fragment comprising amino acids 25-245 of SEQ ID NO: 47 and a light chain comprising SEQ ID NO: 265;
a heavy chain fragment comprising amino acids 25-244 of SEQ ID NO: 49 and a light chain comprising SEQ ID NO: 267;
a heavy chain fragment comprising amino acids 25-249 of SEQ ID NO: 51 and a light chain comprising SEQ ID NO: 269;
a heavy chain fragment comprising amino acids 25-256 of SEQ ID NO: 53 and a light chain comprising SEQ ID NO: 271;
a heavy chain fragment comprising amino acids 25-246 of SEQ ID NO: 55 and a light chain comprising SEQ ID NO: 273;
a heavy chain fragment comprising amino acids 25-251 of SEQ ID NO: 57 and a light chain comprising SEQ ID NO: 275;
a heavy chain fragment comprising amino acids 25-245 of SEQ ID NO: 59 and a light chain comprising SEQ ID NO: 277;
a heavy chain fragment comprising amino acids 25-252 of SEQ ID NO: 61 and a light chain comprising SEQ ID NO: 279;
a heavy chain fragment comprising amino acids 25-246 of SEQ ID NO: 63 and a light chain comprising SEQ ID NO: 281;
a heavy chain fragment comprising amino acids 25-249 of SEQ ID NO: 65 and a light chain comprising SEQ ID NO: 283;
a heavy chain fragment comprising amino acids 25-244 of SEQ ID NO: 67 and a light chain comprising SEQ ID NO: 285;
a heavy chain fragment comprising amino acids 25-249 of SEQ ID NO: 69 and a light chain comprising SEQ ID NO: 287;
a heavy chain fragment comprising amino acids 25-244 of SEQ ID NO: 71 and a light chain comprising SEQ ID NO: 289;
a heavy chain fragment comprising amino acids 25-244 of SEQ ID NO: 73 and a light chain comprising SEQ ID NO: 291;
a heavy chain fragment comprising amino acids 25-249 of SEQ ID NO: 75 and a light chain comprising SEQ ID NO: 293;
a heavy chain fragment comprising amino acids 25-247 of SEQ ID NO: 77 and a light chain comprising SEQ ID NO: 295;
a heavy chain fragment comprising amino acids 25-247 of SEQ ID NO: 79 and a light chain comprising SEQ ID NO: 297;
a heavy chain fragment comprising amino acids 25-255 of SEQ ID NO: 81 and a light chain comprising SEQ ID NO: 299;
a heavy chain fragment comprising amino acids 25-246 of SEQ ID NO: 83 and a light chain comprising SEQ ID NO: 301;
a heavy chain fragment comprising amino acids 25-248 of SEQ ID NO: 85 and a light chain comprising SEQ ID NO: 303;
a heavy chain fragment comprising amino acids 25-252 of SEQ ID NO: 87 and a light chain comprising SEQ ID NO: 305;
a heavy chain fragment comprising amino acids 25-248 of SEQ ID NO: 89 and a light chain comprising SEQ ID NO: 307;
a heavy chain fragment comprising amino acids 25-244 of SEQ ID NO: 91 and a light chain comprising SEQ ID NO: 309;
a heavy chain fragment comprising amino acids 25-247 of SEQ ID NO: 93 and a light chain comprising SEQ ID NO: 311;
a heavy chain fragment comprising amino acids 25-254 of SEQ ID NO: 95 and a light chain comprising SEQ ID NO: 313;
a heavy chain fragment comprising amino acids 25-251 of SEQ ID NO: 97 and a light chain comprising SEQ ID NO: 315;
a heavy chain fragment comprising amino acids 25-245 of SEQ ID NO: 99 and a light chain comprising SEQ ID NO: 317;
a heavy chain fragment comprising amino acids 25-257 of SEQ ID NO: 101 and a light chain comprising SEQ ID NO: 319;
a heavy chain fragment comprising amino acids 25-256 of SEQ ID NO: 103 and a light chain comprising SEQ ID NO: 321;
a heavy chain fragment comprising amino acids 25-249 of SEQ ID NO: 105 and a light chain comprising SEQ ID NO: 323;
a heavy chain fragment comprising amino acids 25-248 of SEQ ID NO: 107 and a light chain comprising SEQ ID NO: 325;
a heavy chain fragment comprising amino acids 25-250 of SEQ ID NO: 109 and a light chain comprising SEQ ID NO: 327;
a heavy chain fragment comprising amino acids 25-244 of SEQ ID NO: 111 and a light chain comprising SEQ ID NO: 329;
a heavy chain fragment comprising amino acids 25-247 of SEQ ID NO: 113 and a light chain comprising SEQ ID NO: 331;
a heavy chain fragment comprising amino acids 25-248 of SEQ ID NO: 115 and a light chain comprising SEQ ID NO: 333;
a heavy chain fragment comprising amino acids 25-245 of SEQ ID NO: 117 and a light chain comprising SEQ ID NO: 335;
a heavy chain fragment comprising amino acids 25-253 of SEQ ID NO: 119 and a light chain comprising SEQ ID NO: 337;
a heavy chain fragment comprising amino acids 25-249 of SEQ ID NO: 121 and a light chain comprising SEQ ID NO: 339;
a heavy chain fragment comprising amino acids 25-253 of SEQ ID NO: 123 and a light chain comprising SEQ ID NO: 341
a heavy chain fragment comprising amino acids 25-253 of SEQ ID NO: 125 and a light chain comprising SEQ ID NO: 343;
a heavy chain fragment comprising amino acids 25-251 of SEQ ID NO: 127 and a light chain comprising SEQ ID NO: 345;
a heavy chain fragment comprising amino acids 25-245 of SEQ ID NO: 129 and a light chain comprising SEQ ID NO: 347;
a heavy chain fragment comprising amino acids 25-249 of SEQ ID NO: 131 and a light chain comprising SEQ ID NO: 349;
a heavy chain fragment comprising amino acids 25-250 of SEQ ID NO: 133 and a light chain comprising SEQ ID NO: 351;
a heavy chain fragment comprising amino acids 25-256 of SEQ ID NO: 135 and a light chain comprising SEQ ID NO: 353;
a heavy chain fragment comprising amino acids 25-250 of SEQ ID NO: 137 and a light chain comprising SEQ ID NO: 355;
a heavy chain fragment comprising amino acids 25-247 of SEQ ID NO: 139 and a light chain comprising SEQ ID NO: 357;
a heavy chain fragment comprising amino acids 25-253 of SEQ ID NO: 141 and a light chain comprising SEQ ID NO: 359;
a heavy chain fragment comprising amino acids 25-249 of SEQ ID NO: 143 and a light chain comprising SEQ ID NO: 361;
a heavy chain fragment comprising amino acids 25-246 of SEQ ID NO: 145 and a light chain comprising SEQ ID NO: 363;
a heavy chain fragment comprising amino acids 25-251 of SEQ ID NO: 147 and a light chain comprising SEQ ID NO: 365;
a heavy chain fragment comprising amino acids 25-245 of SEQ ID NO: 149 and a light chain comprising SEQ ID NO: 367;
a heavy chain fragment comprising amino acids 25-244 of SEQ ID NO: 151 and a light chain comprising SEQ ID NO: 369;
a heavy chain fragment comprising amino acids 25-249 of SEQ ID NO: 153 and a light chain comprising SEQ ID NO: 371;
a heavy chain fragment comprising amino acids 25-257 of SEQ ID NO: 155 and a light chain comprising SEQ ID NO: 373;
a heavy chain fragment comprising amino acids 25-250 of SEQ ID NO: 157 and a light chain comprising SEQ ID NO: 375;
a heavy chain fragment comprising amino acids 25-253 of SEQ ID NO: 159 and a light chain comprising SEQ ID NO: 377;
a heavy chain fragment comprising amino acids 25-245 of SEQ ID NO: 161 and a light chain comprising SEQ ID NO: 379;
a heavy chain fragment comprising amino acids 25-260 of SEQ ID NO: 163 and a light chain comprising SEQ ID NO: 381;
a heavy chain fragment comprising amino acids 25-245 of SEQ ID NO: 165 and a light chain comprising SEQ ID NO: 383;
a heavy chain fragment comprising amino acids 25-244 of SEQ ID NO: 167 and a light chain comprising SEQ ID NO: 385;
a heavy chain fragment comprising amino acids 25-244 of SEQ ID NO: 169 and a light chain comprising SEQ ID NO: 387;
a heavy chain fragment comprising amino acids 25-245 of SEQ ID NO: 171 and a light chain comprising SEQ ID NO: 389;
a heavy chain fragment comprising amino acids 25-244 of SEQ ID NO: 173 and a light chain comprising SEQ ID NO: 391;
a heavy chain fragment comprising amino acids 25-248 of SEQ ID NO: 175 and a light chain comprising SEQ ID NO: 393;
a heavy chain fragment comprising amino acids 25-246 of SEQ ID NO: 177 and a light chain comprising SEQ ID NO: 395;
a heavy chain fragment comprising amino acids 25-249 of SEQ ID NO: 179 and a light chain comprising SEQ ID NO: 397;
a heavy chain fragment comprising amino acids 25-249 of SEQ ID NO: 181 and a light chain comprising SEQ ID NO: 399;
a heavy chain fragment comprising amino acids 25-247 of SEQ ID NO: 183 and a light chain comprising SEQ ID NO: 401;
a heavy chain fragment comprising amino acids 25-251 of SEQ ID NO: 185 and a light chain comprising SEQ ID NO: 403;
a heavy chain fragment comprising amino acids 25-247 of SEQ ID NO: 187 and a light chain comprising SEQ ID NO: 405;
a heavy chain fragment comprising amino acids 25-249 of SEQ ID NO: 189 and a light chain comprising SEQ ID NO: 407;
a heavy chain fragment comprising amino acids 25-248 of SEQ ID NO: 191 and a light chain comprising SEQ ID NO: 409;
a heavy chain fragment comprising amino acids 25-246 of SEQ ID NO: 193 and a light chain comprising SEQ ID NO: 411;
a heavy chain fragment comprising amino acids 25-250 of SEQ ID NO: 195 and a light chain comprising SEQ ID NO: 413;
a heavy chain fragment comprising amino acids 25-245 of SEQ ID NO: 197 and a light chain comprising SEQ ID NO: 415;
a heavy chain fragment comprising amino acids 25-250 of SEQ ID NO: 199 and a light chain comprising SEQ ID NO: 417;
a heavy chain fragment comprising amino acids 25-246 of SEQ ID NO: 201 and a light chain comprising SEQ ID NO: 419;
a heavy chain fragment comprising amino acids 25-243 of SEQ ID NO: 203 and a light chain comprising SEQ ID NO: 421;
a heavy chain fragment comprising amino acids 25-248 of SEQ ID NO: 205 and a light chain comprising SEQ ID NO: 423;
a heavy chain fragment comprising amino acids 25-250 of SEQ ID NO: 207 and a light chain comprising SEQ ID NO: 425;
a heavy chain fragment comprising amino acids 25-249 of SEQ ID NO: 209 and a light chain comprising SEQ ID NO: 427;
a heavy chain fragment comprising amino acids 25-255 of SEQ ID NO: 211 and a light chain comprising SEQ ID NO: 429;
a heavy chain fragment comprising amino acids 25-245 of SEQ ID NO: 213 and a light chain comprising SEQ ID NO: 431;
a heavy chain fragment comprising amino acids 25-244 of SEQ ID NO: 215 and a light chain comprising SEQ ID NO: 433;
a heavy chain fragment comprising amino acids 25-250 of SEQ ID NO: 217 and a light chain comprising SEQ ID NO: 435;
a heavy chain fragment comprising amino acids 25-249 of SEQ ID NO: 219 and a light chain comprising SEQ ID NO: 437;
a heavy chain fragment comprising amino acids 25-247 of SEQ ID NO: 221 and a light chain comprising SEQ ID NO: 439;
a heavy chain fragment comprising amino acids 25-254 of SEQ ID NO: 223 and a light chain comprising SEQ ID NO: 441;
a heavy chain fragment comprising amino acids 25-248 of SEQ ID NO: 225 and a light chain comprising SEQ ID NO: 443;
a heavy chain fragment comprising amino acids 25-249 of SEQ ID NO: 227 and a light chain comprising SEQ ID NO: 445;
a heavy chain fragment comprising amino acids 25-253 of SEQ ID NO: 229 and a light chain comprising SEQ ID NO: 447;
a heavy chain fragment comprising amino acids 25-251 of SEQ ID NO: 231 and a light chain comprising SEQ ID NO: 449;
a heavy chain fragment comprising amino acids 25-244 of SEQ ID NO: 233 and a light chain comprising SEQ ID NO: 451;
a heavy chain fragment comprising amino acids 25-245 of SEQ ID NO: 235 and a light chain comprising SEQ ID NO: 453;
a heavy chain fragment comprising amino acids 25-244 of SEQ ID NO: 237 and a light chain comprising SEQ ID NO: 455; or
a heavy chain fragment comprising amino acids 25-254 of SEQ ID NO: 239 and a light chain comprising SEQ ID NO: 457.

In certain embodiments, the antibody or antigen-binding fragment comprises:

(i) a heavy chain amino acid sequence selected from the group consisting of:

### SEQ ID NO: 23, SEQ ID NO: 25, SEQ ID NO: 27, SEQ ID NO: 29, SEQ ID NO: 31,

### SEQ ID NO: 33, SEQ ID NO: 35, SEQ ID NO: 37, SEQ ID NO: 39, SEQ ID NO: 41,

### SEQ ID NO: 43, SEQ ID NO: 45, SEQ ID NO: 47, SEQ ID NO: 49, SEQ ID NO: 51,

### SEQ ID NO: 53, SEQ ID NO: 55, SEQ ID NO: 57, SEQ ID NO: 59, SEQ ID NO: 61,

### SEQ ID NO: 63, SEQ ID NO: 65, SEQ ID NO: 67, SEQ ID NO: 69, SEQ ID NO: 71,

### SEQ ID NO: 73, SEQ ID NO: 75, SEQ ID NO: 77, SEQ ID NO: 79, SEQ ID NO: 81,

### SEQ ID NO: 83, SEQ ID NO: 85, SEQ ID NO: 87, SEQ ID NO: 89, SEQ ID NO: 91,

### SEQ ID NO: 93, SEQ ID NO: 95, SEQ ID NO: 97, SEQ ID NO: 99, SEQ ID NO: 101,

### SEQ ID NO: 103, SEQ ID NO: 105, SEQ ID NO: 107, SEQ ID NO: 109, SEQ ID NO: 111,

### SEQ ID NO: 113, SEQ ID NO: 115, SEQ ID NO: 117, SEQ ID NO: 119, SEQ ID NO: 121,

### SEQ ID NO: 123, SEQ ID NO: 125, SEQ ID NO: 127, SEQ ID NO: 129, SEQ ID NO: 131,

### SEQ ID NO: 133, SEQ ID NO: 135, SEQ ID NO: 137, SEQ ID NO: 139, SEQ ID NO: 141,

### SEQ ID NO: 143, SEQ ID NO: 145, SEQ ID NO: 147, SEQ ID NO: 149, SEQ ID NO: 151,

### SEQ ID NO: 153, SEQ ID NO: 155, SEQ ID NO: 157, SEQ ID NO: 159, SEQ ID NO: 161,

### SEQ ID NO: 163, SEQ ID NO: 165, SEQ ID NO: 167, SEQ ID NO: 169, SEQ ID NO: 171,

### SEQ ID NO: 173, SEQ ID NO: 175, SEQ ID NO: 177, SEQ ID NO: 179, SEQ ID NO: 181,

### SEQ ID NO: 183, SEQ ID NO: 185, SEQ ID NO: 187, SEQ ID NO: 189, SEQ ID NO: 191,

### SEQ ID NO: 193, SEQ ID NO: 195, SEQ ID NO: 197, SEQ ID NO: 199, SEQ ID NO: 201,

### SEQ ID NO: 203, SEQ ID NO: 205, SEQ ID NO: 207, SEQ ID NO: 209, SEQ ID NO: 211,

### SEQ ID NO: 213, SEQ ID NO: 215, SEQ ID NO: 217, SEQ ID NO: 219, SEQ ID NO: 221,

### SEQ ID NO: 223, SEQ ID NO: 225, SEQ ID NO: 227, SEQ ID NO: 229, SEQ ID NO: 231,

### SEQ ID NO: 233, SEQ ID NO: 235, SEQ ID NO: 237 and SEQ ID NO: 239; and

(ii) a light chain amino acid sequence selected from the group consisting of:

### SEQ ID NO: 241, SEQ ID NO: 243, SEQ ID NO: 245, SEQ ID NO: 247, SEQ ID NO: 249,

### SEQ ID NO: 251, SEQ ID NO: 253, SEQ ID NO: 255, SEQ ID NO: 257, SEQ ID NO: 259,

### SEQ ID NO: 261, SEQ ID NO: 263, SEQ ID NO: 265, SEQ ID NO: 267, SEQ ID NO: 269,

### SEQ ID NO: 271, SEQ ID NO: 273, SEQ ID NO: 275, SEQ ID NO: 277, SEQ ID NO: 279,

### SEQ ID NO: 281, SEQ ID NO: 283, SEQ ID NO: 285, SEQ ID NO: 287, SEQ ID NO: 289,

### SEQ ID NO: 291, SEQ ID NO: 293, SEQ ID NO: 295, SEQ ID NO: 297, SEQ ID NO: 299,

### SEQ ID NO: 301, SEQ ID NO: 303, SEQ ID NO: 305, SEQ ID NO: 307, SEQ ID NO: 309,

### SEQ ID NO: 311, SEQ ID NO: 313, SEQ ID NO: 315, SEQ ID NO: 317, SEQ ID NO: 319,

### SEQ ID NO: 321, SEQ ID NO: 323, SEQ ID NO: 325, SEQ ID NO: 327, SEQ ID NO: 329,

### SEQ ID NO: 331, SEQ ID NO: 333, SEQ ID NO: 335, SEQ ID NO: 337, SEQ ID NO: 339,

### SEQ ID NO: 341, SEQ ID NO: 343, SEQ ID NO: 345, SEQ ID NO: 347, SEQ ID NO: 349,

### SEQ ID NO: 351, SEQ ID NO: 353, SEQ ID NO: 355, SEQ ID NO: 357, SEQ ID NO: 359,

### SEQ ID NO: 361, SEQ ID NO: 363, SEQ ID NO: 365, SEQ ID NO: 367, SEQ ID NO: 369,

### SEQ ID NO: 371, SEQ ID NO: 373, SEQ ID NO: 375, SEQ ID NO: 377, SEQ ID NO: 379,

### SEQ ID NO: 381, SEQ ID NO: 383, SEQ ID NO: 385, SEQ ID NO: 387, SEQ ID NO: 389,

### SEQ ID NO: 391, SEQ ID NO: 393, SEQ ID NO: 395, SEQ ID NO: 397, SEQ ID NO: 399,

### SEQ ID NO: 401, SEQ ID NO: 403, SEQ ID NO: 405, SEQ ID NO: 407, SEQ ID NO: 409,

### SEQ ID NO: 411, SEQ ID NO: 413, SEQ ID NO: 415, SEQ ID NO: 417, SEQ ID NO: 419,

### SEQ ID NO: 421, SEQ ID NO: 423, SEQ ID NO: 425, SEQ ID NO: 427, SEQ ID NO: 429,

### SEQ ID NO: 431, SEQ ID NO: 433, SEQ ID NO: 435, SEQ ID NO: 437, SEQ ID NO: 439,

### SEQ ID NO: 441, SEQ ID NO: 443, SEQ ID NO: 445, SEQ ID NO: 447, SEQ ID NO: 449,

### SEQ ID NO: 451, SEQ ID NO: 453, SEQ ID NO: 455 and SEQ ID NO: 457.

In certain aspects the disclosure relates to an isolated antibody or an antigen-binding fragment thereof that specifically binds ST2, comprising:

(i) a heavy chain variable domain comprising complementarity determining regions (CDRs) as set forth in a heavy chain variable domain amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239; and
(ii) a light chain variable domain comprising CDRs as set forth in a light chain variable region amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457.

In certain aspects the disclosure relates to a recombinant antibody or an antigen-binding fragment thereof that specifically binds ST2, wherein the antibody, or antigen-binding fragment thereof, comprises six complementarity determining regions (CDRs): CDRH1, CDRH2, CDRH3, CDRL1, CDRL2, and CDRL3,

wherein CDRH1 comprises an amino acid sequence that has at least 85%, 90%, 95%, 96%, 97%, 98%, or 99% sequence identity to a CDRH1 amino acid sequence selected from the group consisting of:

### SEQ ID NO: 458, SEQ ID NO: 464, SEQ ID NO: 470, SEQ ID NO: 476, SEQ ID NO: 482,

### SEQ ID NO: 488, SEQ ID NO: 494, SEQ ID NO: 500, SEQ ID NO: 506, SEQ ID NO: 512,

### SEQ ID NO: 518, SEQ ID NO: 524, SEQ ID NO: 530, SEQ ID NO: 536, SEQ ID NO: 542,

### SEQ ID NO: 548, SEQ ID NO: 554, SEQ ID NO: 560, SEQ ID NO: 566, SEQ ID NO: 572,

### SEQ ID NO: 578, SEQ ID NO: 584, SEQ ID NO: 590, SEQ ID NO: 596, SEQ ID NO: 602,

### SEQ ID NO: 608, SEQ ID NO: 614, SEQ ID NO: 620, SEQ ID NO: 626, SEQ ID NO: 632,

### SEQ ID NO: 638, SEQ ID NO: 644, SEQ ID NO: 650, SEQ ID NO: 656, SEQ ID NO: 662,

### SEQ ID NO: 668, SEQ ID NO: 674, SEQ ID NO: 680, SEQ ID NO: 686, SEQ ID NO: 692,

### SEQ ID NO: 698, SEQ ID NO: 704, SEQ ID NO: 710, SEQ ID NO: 716, SEQ ID NO: 722,

### SEQ ID NO: 728, SEQ ID NO: 734, SEQ ID NO: 740, SEQ ID NO: 746, SEQ ID NO: 752,

### SEQ ID NO: 758, SEQ ID NO: 764, SEQ ID NO: 770, SEQ ID NO: 776, SEQ ID NO: 782,

### SEQ ID NO: 788, SEQ ID NO: 794, SEQ ID NO: 800, SEQ ID NO: 806, SEQ ID NO: 812,

### SEQ ID NO: 818, SEQ ID NO: 824, SEQ ID NO: 830, SEQ ID NO: 836, SEQ ID NO: 842,

### SEQ ID NO: 848, SEQ ID NO: 854, SEQ ID NO: 860, SEQ ID NO: 866, SEQ ID NO: 872,

### SEQ ID NO: 878, SEQ ID NO: 884, SEQ ID NO: 890, SEQ ID NO: 896, SEQ ID NO: 902,

### SEQ ID NO: 908, SEQ ID NO: 914, SEQ ID NO: 920, SEQ ID NO: 926, SEQ ID NO: 932,

### SEQ ID NO: 938, SEQ ID NO: 944, SEQ ID NO: 950, SEQ ID NO: 956, SEQ ID NO: 962,

### SEQ ID NO: 968, SEQ ID NO: 974, SEQ ID NO: 980, SEQ ID NO: 986, SEQ ID NO: 992,

### SEQ ID NO: 998, SEQ ID NO: 1004, SEQ ID NO: 1010, SEQ ID NO: 1016, SEQ ID NO: 1022,

### SEQ ID NO: 1028, SEQ ID NO: 1034, SEQ ID NO: 1040, SEQ ID NO: 1046, SEQ ID NO: 1052,

### SEQ ID NO: 1058, SEQ ID NO: 1064, SEQ ID NO: 1070, SEQ ID NO: 1076, SEQ ID NO: 1082,

### SEQ ID NO: 1088, SEQ ID NO: 1094, SEQ ID NO: 1100 and SEQ ID NO: 1106;

wherein CDRH2 comprises an amino acid sequence that has at least 85%, 90%, 95%, 96%, 97%, 98%, or 99% sequence identity to a CDRH2 amino acid sequence selected from the group consisting of:

### SEQ ID NO: 459, SEQ ID NO: 465, SEQ ID NO: 471, SEQ ID NO: 477, SEQ ID NO: 483,

### SEQ ID NO: 489, SEQ ID NO: 495, SEQ ID NO: 501, SEQ ID NO: 507, SEQ ID NO: 513,

### SEQ ID NO: 519, SEQ ID NO: 525, SEQ ID NO: 531, SEQ ID NO: 537, SEQ ID NO: 543,

### SEQ ID NO: 549, SEQ ID NO: 555, SEQ ID NO: 561, SEQ ID NO: 567, SEQ ID NO: 573,

### SEQ ID NO: 579, SEQ ID NO: 585, SEQ ID NO: 591, SEQ ID NO: 597, SEQ ID NO: 603,

### SEQ ID NO: 609, SEQ ID NO: 615, SEQ ID NO: 621, SEQ ID NO: 627, SEQ ID NO: 633,

### SEQ ID NO: 639, SEQ ID NO: 645, SEQ ID NO: 651, SEQ ID NO: 657, SEQ ID NO: 663,

### SEQ ID NO: 669, SEQ ID NO: 675, SEQ ID NO: 681, SEQ ID NO: 687, SEQ ID NO: 693,

### SEQ ID NO: 699, SEQ ID NO: 705, SEQ ID NO: 711, SEQ ID NO: 717, SEQ ID NO: 723,

### SEQ ID NO: 729, SEQ ID NO: 735, SEQ ID NO: 741, SEQ ID NO: 747, SEQ ID NO: 753,

### SEQ ID NO: 759, SEQ ID NO: 765, SEQ ID NO: 771, SEQ ID NO: 777, SEQ ID NO: 783,

### SEQ ID NO: 789, SEQ ID NO: 795, SEQ ID NO: 801, SEQ ID NO: 807, SEQ ID NO: 813,

### SEQ ID NO: 819, SEQ ID NO: 825, SEQ ID NO: 831, SEQ ID NO: 837, SEQ ID NO: 843,

### SEQ ID NO: 849, SEQ ID NO: 855, SEQ ID NO: 861, SEQ ID NO: 867, SEQ ID NO: 873,

### SEQ ID NO: 879, SEQ ID NO: 885, SEQ ID NO: 891, SEQ ID NO: 897, SEQ ID NO: 903,

### SEQ ID NO: 909, SEQ ID NO: 915, SEQ ID NO: 921, SEQ ID NO: 927, SEQ ID NO: 933,

### SEQ ID NO: 939, SEQ ID NO: 945, SEQ ID NO: 951, SEQ ID NO: 957, SEQ ID NO: 963,

### SEQ ID NO: 969, SEQ ID NO: 975, SEQ ID NO: 981, SEQ ID NO: 987, SEQ ID NO: 993,

### SEQ ID NO: 999, SEQ ID NO: 1005, SEQ ID NO: 1011, SEQ ID NO: 1017, SEQ ID NO: 1023,

### SEQ ID NO: 1029, SEQ ID NO: 1035, SEQ ID NO: 1041, SEQ ID NO: 1047, SEQ ID NO: 1053,

### SEQ ID NO: 1059, SEQ ID NO: 1065, SEQ ID NO: 1071, SEQ ID NO: 1077, SEQ ID NO: 1083,

### SEQ ID NO: 1089, SEQ ID NO: 1095, SEQ ID NO: 1101 and SEQ ID NO: 1107,

wherein CDRH3 comprises an amino acid sequence that has at least 85%, 90%, 95%, 96%, 97%, 98% or 99% sequence identity to a CDRH3 amino acid sequence selected from the group consisting of:

### SEQ ID NO: 460, SEQ ID NO: 466, SEQ ID NO: 472, SEQ ID NO: 478, SEQ ID NO: 484,

### SEQ ID NO: 490, SEQ ID NO: 496, SEQ ID NO: 502, SEQ ID NO: 508, SEQ ID NO: 514,

### SEQ ID NO: 520, SEQ ID NO: 526, SEQ ID NO: 532, SEQ ID NO: 538, SEQ ID NO: 544,

### SEQ ID NO: 550, SEQ ID NO: 556, SEQ ID NO: 562, SEQ ID NO: 568, SEQ ID NO: 574,

### SEQ ID NO: 580, SEQ ID NO: 586, SEQ ID NO: 592, SEQ ID NO: 598, SEQ ID NO: 604,

### SEQ ID NO: 610, SEQ ID NO: 616, SEQ ID NO: 622, SEQ ID NO: 628, SEQ ID NO: 634,

### SEQ ID NO: 640, SEQ ID NO: 646, SEQ ID NO: 652, SEQ ID NO: 658, SEQ ID NO: 664,

### SEQ ID NO: 670, SEQ ID NO: 676, SEQ ID NO: 682, SEQ ID NO: 688, SEQ ID NO: 694,

### SEQ ID NO: 700, SEQ ID NO: 706, SEQ ID NO: 712, SEQ ID NO: 718, SEQ ID NO: 724,

### SEQ ID NO: 730, SEQ ID NO: 736, SEQ ID NO: 742, SEQ ID NO: 748, SEQ ID NO: 754,

### SEQ ID NO: 760, SEQ ID NO: 766, SEQ ID NO: 772, SEQ ID NO: 778, SEQ ID NO: 784,

### SEQ ID NO: 790, SEQ ID NO: 796, SEQ ID NO: 802, SEQ ID NO: 808, SEQ ID NO: 814,

### SEQ ID NO: 820, SEQ ID NO: 826, SEQ ID NO: 832, SEQ ID NO: 838, SEQ ID NO: 844,

### SEQ ID NO: 850, SEQ ID NO: 856, SEQ ID NO: 862, SEQ ID NO: 868, SEQ ID NO: 874,

### SEQ ID NO: 880, SEQ ID NO: 886, SEQ ID NO: 892, SEQ ID NO: 898, SEQ ID NO: 904,

### SEQ ID NO: 910, SEQ ID NO: 916, SEQ ID NO: 922, SEQ ID NO: 928, SEQ ID NO: 934,

### SEQ ID NO: 940, SEQ ID NO: 946, SEQ ID NO: 952, SEQ ID NO: 958, SEQ ID NO: 964,

### SEQ ID NO: 970, SEQ ID NO: 976, SEQ ID NO: 982, SEQ ID NO: 988, SEQ ID NO: 994,

### SEQ ID NO: 1000, SEQ ID NO: 1006, SEQ ID NO: 1012, SEQ ID NO: 1018, SEQ ID NO: 1024,

### SEQ ID NO: 1030, SEQ ID NO: 1036, SEQ ID NO: 1042, SEQ ID NO: 1048, SEQ ID NO: 1054,

### SEQ ID NO: 1060, SEQ ID NO: 1066, SEQ ID NO: 1072, SEQ ID NO: 1078, SEQ ID NO: 1084,

### SEQ ID NO: 1090, SEQ ID NO: 1096, SEQ ID NO: 1102 and SEQ ID NO: 1108,

wherein CDRL1 comprises an amino acid sequence that has at least 85%, 90%, 95%, 96%, 97%, 98%, or 99% sequence identity to a CDRL1 amino acid sequence selected from the group consisting of:

### SEQ ID NO: 461, SEQ ID NO: 467, SEQ ID NO: 473, SEQ ID NO: 479, SEQ ID NO: 485,

### SEQ ID NO: 491, SEQ ID NO: 497, SEQ ID NO: 503, SEQ ID NO: 509, SEQ ID NO: 515,

### SEQ ID NO: 521, SEQ ID NO: 527, SEQ ID NO: 533, SEQ ID NO: 539, SEQ ID NO: 545,

### SEQ ID NO: 551, SEQ ID NO: 557, SEQ ID NO: 563, SEQ ID NO: 569, SEQ ID NO: 575,

### SEQ ID NO: 581, SEQ ID NO: 587, SEQ ID NO: 593, SEQ ID NO: 599, SEQ ID NO: 605,

### SEQ ID NO: 611, SEQ ID NO: 617, SEQ ID NO: 623, SEQ ID NO: 629, SEQ ID NO: 635,

### SEQ ID NO: 641, SEQ ID NO: 647, SEQ ID NO: 653, SEQ ID NO: 659, SEQ ID NO: 665,

### SEQ ID NO: 671, SEQ ID NO: 677, SEQ ID NO: 683, SEQ ID NO: 689, SEQ ID NO: 695,

### SEQ ID NO: 701, SEQ ID NO: 707, SEQ ID NO: 713, SEQ ID NO: 719, SEQ ID NO: 725,

### SEQ ID NO: 731, SEQ ID NO: 737, SEQ ID NO: 743, SEQ ID NO: 749, SEQ ID NO: 755,

### SEQ ID NO: 761, SEQ ID NO: 767, SEQ ID NO: 773, SEQ ID NO: 779, SEQ ID NO: 785,

### SEQ ID NO: 791, SEQ ID NO: 797, SEQ ID NO: 803, SEQ ID NO: 809, SEQ ID NO: 815,

### SEQ ID NO: 821, SEQ ID NO: 827, SEQ ID NO: 833, SEQ ID NO: 839, SEQ ID NO: 845,

### SEQ ID NO: 851, SEQ ID NO: 857, SEQ ID NO: 863, SEQ ID NO: 869, SEQ ID NO: 875,

### SEQ ID NO: 881, SEQ ID NO: 887, SEQ ID NO: 893, SEQ ID NO: 899, SEQ ID NO: 905,

### SEQ ID NO: 911, SEQ ID NO: 917, SEQ ID NO: 923, SEQ ID NO: 929, SEQ ID NO: 935,

### SEQ ID NO: 941, SEQ ID NO: 947, SEQ ID NO: 953, SEQ ID NO: 959, SEQ ID NO: 965,

### SEQ ID NO: 971, SEQ ID NO: 977, SEQ ID NO: 983, SEQ ID NO: 989, SEQ ID NO: 995,

### SEQ ID NO: 1001, SEQ ID NO: 1007, SEQ ID NO: 1013, SEQ ID NO: 1019, SEQ ID NO: 1025,

### SEQ ID NO: 1031, SEQ ID NO: 1037, SEQ ID NO: 1043, SEQ ID NO: 1049, SEQ ID NO: 1055,

### SEQ ID NO: 1061, SEQ ID NO: 1067, SEQ ID NO: 1073, SEQ ID NO: 1079, SEQ ID NO: 1085,

### SEQ ID NO: 1091, SEQ ID NO: 1097, SEQ ID NO: 1103 and SEQ ID NO: 1109,

wherein CDRL2 comprises an amino acid sequence that that has at least 85%, 90%, 95%, 96%, 97%, 98%, or 99% sequence identity to a CDRL2 sequence selected from the group consisting of:

### SEQ ID NO: 462, SEQ ID NO: 468, SEQ ID NO: 474, SEQ ID NO: 480, SEQ ID NO: 486,

### SEQ ID NO: 492, SEQ ID NO: 498, SEQ ID NO: 504, SEQ ID NO: 510, SEQ ID NO: 516,

### SEQ ID NO: 522, SEQ ID NO: 528, SEQ ID NO: 534, SEQ ID NO: 540, SEQ ID NO: 546,

### SEQ ID NO: 552, SEQ ID NO: 558, SEQ ID NO: 564, SEQ ID NO: 570, SEQ ID NO: 576,

### SEQ ID NO: 582, SEQ ID NO: 588, SEQ ID NO: 594, SEQ ID NO: 600, SEQ ID NO: 606,

### SEQ ID NO: 612, SEQ ID NO: 618, SEQ ID NO: 624, SEQ ID NO: 630, SEQ ID NO: 636,

### SEQ ID NO: 642, SEQ ID NO: 648, SEQ ID NO: 654, SEQ ID NO: 660, SEQ ID NO: 666,

### SEQ ID NO: 672, SEQ ID NO: 678, SEQ ID NO: 684, SEQ ID NO: 690, SEQ ID NO: 696,

### SEQ ID NO: 702, SEQ ID NO: 708, SEQ ID NO: 714, SEQ ID NO: 720, SEQ ID NO: 726,

### SEQ ID NO: 732, SEQ ID NO: 738, SEQ ID NO: 744, SEQ ID NO: 750, SEQ ID NO: 756,

### SEQ ID NO: 762, SEQ ID NO: 768, SEQ ID NO: 774, SEQ ID NO: 780, SEQ ID NO: 786,

### SEQ ID NO: 792, SEQ ID NO: 798, SEQ ID NO: 804, SEQ ID NO: 810, SEQ ID NO: 816,

### SEQ ID NO: 822, SEQ ID NO: 828, SEQ ID NO: 834, SEQ ID NO: 840, SEQ ID NO: 846,

### SEQ ID NO: 852, SEQ ID NO: 858, SEQ ID NO: 864, SEQ ID NO: 870, SEQ ID NO: 876,

### SEQ ID NO: 882, SEQ ID NO: 888, SEQ ID NO: 894, SEQ ID NO: 900, SEQ ID NO: 906,

### SEQ ID NO: 912, SEQ ID NO: 918, SEQ ID NO: 924, SEQ ID NO: 930, SEQ ID NO: 936,

### SEQ ID NO: 942, SEQ ID NO: 948, SEQ ID NO: 954, SEQ ID NO: 960, SEQ ID NO: 966,

### SEQ ID NO: 972, SEQ ID NO: 978, SEQ ID NO: 984, SEQ ID NO: 990, SEQ ID NO: 996,

### SEQ ID NO: 1002, SEQ ID NO: 1008, SEQ ID NO: 1014, SEQ ID NO: 1020, SEQ ID NO: 1026,

### SEQ ID NO: 1032, SEQ ID NO: 1038, SEQ ID NO: 1044, SEQ ID NO: 1050, SEQ ID NO: 1056,

### SEQ ID NO: 1062, SEQ ID NO: 1068, SEQ ID NO: 1074, SEQ ID NO: 1080, SEQ ID NO: 1086,

### SEQ ID NO: 1092, SEQ ID NO: 1098, SEQ ID NO: 1104 and SEQ ID NO: 1110,

wherein CDRL3 comprises an amino acid sequence that has at least 85%, 90%, 95%, 96%, 97%, 98%, or 99% sequence identity to a CDRL3 sequence selected from the group consisting of:

### SEQ ID NO: 463, SEQ ID NO: 469, SEQ ID NO: 475, SEQ ID NO: 481, SEQ ID NO: 487,

### SEQ ID NO: 493, SEQ ID NO: 499, SEQ ID NO: 505, SEQ ID NO: 511, SEQ ID NO: 517,

### SEQ ID NO: 523, SEQ ID NO: 529, SEQ ID NO: 535, SEQ ID NO: 541, SEQ ID NO: 547,

### SEQ ID NO: 553, SEQ ID NO: 559, SEQ ID NO: 565, SEQ ID NO: 571, SEQ ID NO: 577,

### SEQ ID NO: 583, SEQ ID NO: 589, SEQ ID NO: 595, SEQ ID NO: 601, SEQ ID NO: 607,

### SEQ ID NO: 613, SEQ ID NO: 619, SEQ ID NO: 625, SEQ ID NO: 631, SEQ ID NO: 637,

### SEQ ID NO: 643, SEQ ID NO: 649, SEQ ID NO: 655, SEQ ID NO: 661, SEQ ID NO: 667,

### SEQ ID NO: 673, SEQ ID NO: 679, SEQ ID NO: 685, SEQ ID NO: 691, SEQ ID NO: 697,

### SEQ ID NO: 703, SEQ ID NO: 709, SEQ ID NO: 715, SEQ ID NO: 721, SEQ ID NO: 727,

### SEQ ID NO: 733, SEQ ID NO: 739, SEQ ID NO: 745, SEQ ID NO: 751, SEQ ID NO: 757,

### SEQ ID NO: 763, SEQ ID NO: 769, SEQ ID NO: 775, SEQ ID NO: 781, SEQ ID NO: 787,

### SEQ ID NO: 793, SEQ ID NO: 799, SEQ ID NO: 805, SEQ ID NO: 811, SEQ ID NO: 817,

### SEQ ID NO: 823, SEQ ID NO: 829, SEQ ID NO: 835, SEQ ID NO: 841, SEQ ID NO: 847,

### SEQ ID NO: 853, SEQ ID NO: 859, SEQ ID NO: 865, SEQ ID NO: 871, SEQ ID NO: 877,

### SEQ ID NO: 883, SEQ ID NO: 889, SEQ ID NO: 895, SEQ ID NO: 901, SEQ ID NO: 907,

### SEQ ID NO: 913, SEQ ID NO: 919, SEQ ID NO: 925, SEQ ID NO: 931, SEQ ID NO: 937,

### SEQ ID NO: 943, SEQ ID NO: 949, SEQ ID NO: 955, SEQ ID NO: 961, SEQ ID NO: 967,

### SEQ ID NO: 973, SEQ ID NO: 979, SEQ ID NO: 985, SEQ ID NO: 991, SEQ ID NO: 997,

### SEQ ID NO: 1003, SEQ ID NO: 1009, SEQ ID NO: 1015, SEQ ID NO: 1021, SEQ ID NO: 1027,

### SEQ ID NO: 1033, SEQ ID NO: 1039, SEQ ID NO: 1045, SEQ ID NO: 1051, SEQ ID NO: 1057,

### SEQ ID NO: 1063, SEQ ID NO: 1069, SEQ ID NO: 1075, SEQ ID NO: 1081, SEQ ID NO: 1087,

### SEQ ID NO: 1093, SEQ ID NO: 1099, SEQ ID NO: 1105 and SEQ ID NO: 1111.

In certain embodiments, the recombinant antibody, or the antigen-binding fragment thereof is a fully human antibody. In certain embodiments, the antigen-binding fragment is selected from the group consisting of a Fab fragment, a F(ab′)2 fragment, a Fd fragment, a Fv fragment, a dAb fragment, a single chain Fv (scFv), a dimerized variable region (V region) fragment (diabody), and a disulfide-stabilized V region fragment (dsFv).

In certain aspects the disclosure relates to a fusion protein comprising: a) a human IL-2 protein domain; b) an immunoglobulin Fc protein domain; and c) an ST2-binding domain comprising an antibody specific for ST2, or an antigen-binding fragment thereof as disclosed herein. In certain embodiments of the fusion proteins disclosed herein, the fusion protein further comprises at least one peptide linker domain. In certain embodiments of the fusion proteins disclosed herein, the human IL-2 protein domain comprises human IL-2 with a substitution selected from the group consisting of: T3A, N88R, N88G, D20H, C125S, Q126L, and Q126F, relative to the amino acid sequence of SEQ ID NO: 2. In certain embodiments of the fusion proteins disclosed herein, the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9. In certain embodiments of the fusion proteins disclosed herein, the peptide linker domain comprises the amino acid sequence of SEQ ID NO: 6. In certain embodiments of the fusion proteins disclosed herein, the fusion protein comprises a first peptide linker domain and a second peptide linker domain.

In certain embodiments of the fusion proteins disclosed herein, each domain has an amino-terminus (N-terminus) and a carboxy terminus (C-terminus); and wherein the fusion protein is configured so that

a) the C-terminus of the human IL-2 protein domain is fused through a peptide bond to the N-terminus of the first peptide linker domain;
b) the N-terminus of the IgG Fc protein domain is fused through a peptide bond to the C-terminus of the first peptide linker domain;
c) the N-terminus of the second peptide linker domain is fused through a peptide bond to the C-terminus of the IgG Fc protein domain; and
d) the N-terminus of the ST2-binding domain is fused through a peptide bond to the C-terminus of the second peptide linker domain.

In certain aspects the disclosure relates to a nucleic acid encoding a fusion protein as disclosed herein. In certain aspects the disclosure relates to a dimeric protein comprising a fusion protein as disclosed herein.

In certain aspects the disclosure relates to a dimeric protein comprising a first fusion protein and a second fusion protein, wherein:
a. each fusion protein comprises an immunoglobulin (IgG) Fc protein domain and at least one additional protein domain selected from the group consisting of
   i. a human IL-2 protein domain; and
   ii. an ST2-binding domain comprising an antibody or antigen-binding fragment as disclosed herein; and
b. the dimeric protein comprises at least one of the human IL-2 protein domain and at least one of the ST2-binding domain.

In certain embodiments of the dimeric proteins disclosed herein,
a. the first fusion protein comprises the human IL-2 protein domain, a first immunoglobulin Fc protein domain, and a first peptide linker; and
b. the second fusion protein comprises the ST2-binding domain, a second immunoglobulin Fc protein domain, and a second peptide linker domain.

In certain embodiments of the dimeric proteins disclosed herein,
a. each domain has an amino-terminus (N-terminus) and a carboxy terminus (C-terminus);
b. the first fusion protein is configured so that
   i. the C-terminus of the human IL-2 protein domain is fused through a peptide bond to the N-terminus of the first peptide linker domain; and
   ii. the N-terminus of the first IgG Fc protein domain is fused through a peptide bond to the C-terminus of the first peptide linker domain; and
c. the second fusion protein is configured so that
   i. the C-terminus of the second IgG Fc protein domain is fused through a peptide bond to the N-terminus of the second peptide linker domain; and
   ii. the N-terminus of the ST2-binding domain is fused through a peptide bond to the C-terminus of the second peptide linker domain.

In certain embodiments of the dimeric proteins disclosed herein, at least one of the fusion proteins further comprises at least one peptide linker domain. In certain embodiments of the dimeric proteins disclosed herein, the peptide linker domain comprises the amino acid sequence of SEQ ID NO: 6.

In certain embodiments, the human IL-2 protein domain comprises human IL-2 with a substitution selected from the group consisting of: T3A, N88R, N88G, D20H, C125S, Q126L, and Q126F, relative to the amino acid sequence of SEQ ID NO: 2. In certain embodiments of the dimeric proteins disclosed herein, the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9.

In certain aspects the disclosure relates to a pharmaceutical composition comprising a dimeric protein as disclosed herein.

In certain aspects the disclosure relates to a method for treating a condition, the method comprising administering to a subject in need thereof a therapeutically-effective amount of a pharmaceutical composition as disclosed herein. In certain embodiments, the condition is selected from the group consisting of an inflammatory myopathy, muscular dystrophy, polymyositis, dermatomyositis, an inflammatory condition of adipose tissue, an inflammatory condition of the colon, an inflammatory condition of the lung, Graft-vs-Host Disease, Pemphigus Vulgaris, Systemic Lupus Erythematosus, Scleroderma, Ulcerative Colitis, Crohn's Disease, Psoriasis, Type 1 Diabetes, Multiple Sclerosis, Amyotrophic Lateral Sclerosis, Alopecia Areata, Uveitis, Neuromyelitis Optica, and Duchenne Muscular Dystrophy. In certain embodiments, the administration is subcutaneous.

In certain aspects the disclosure relates to a method of selectively activating an ST2⁺ regulatory T cell relative to an ST2⁻ regulatory T cell in a subject, the method comprising administering to the subject a therapeutically effective amount of a pharmaceutical composition as disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a diagram illustrating the overlap of cells expressing IL-2Rαβγ and ST2.

FIG. 2A shows a schematic diagram of a compound having an IL2R-binding moiety, an ST2-binding moiety, and a linker between them.

FIG. 2B shows a schematic diagram of an exemplary compound having an IL2R-binding moiety covalently linked to a multimerization moiety, an ST2-binding moiety covalently linked to a multimerization moiety, and covalent bonds between the multimerization moieties.

FIG. 2C shows a schematic diagram of an exemplary compound having an IL2R-binding moiety covalently linked to a multimerization moiety, an ST2-binding moiety covalently linked to a linker covalently linked to a multimerization moiety, and covalent bonds between the multimerization moieties.

FIG. 2D shows a schematic diagram of an exemplary compound having an IL2R-binding moiety covalently linked to a linker covalently linked to a multimerization moiety, an ST2-binding moiety covalently linked to a multimerization moiety, and covalent bonds between the multimerization moieties.

FIG. 2E shows a schematic diagram of an exemplary compound having an IL2R-binding moiety covalently linked to a linker covalently linked to a multimerization moiety, an ST2-binding moiety covalently linked to a linker covalently linked to a multimerization moiety, and covalent bonds between the multimerization moieties.

FIG. 2F shows a schematic diagram of an exemplary compound having a) an IL2R-binding moiety covalently linked to a multimerization moiety covalently linked to an ST2-binding moiety, b) an ST2-binding moiety covalently linked to a multimerization moiety covalently linked to an IL2R-binding moiety, and c) covalent bonds between the multimerization moieties.

FIG. 3A-3E show schematic diagrams of dimeric proteins comprising IgG1 Fc regions. The dimeric proteins comprise an IL-2 variant (N88R, C125S) and an antigen-binding fragment (Fab) that binds to ST2 (Ab2 or Ab4). Each diagram represents two different dimeric proteins, one containing Ab2 as the Fab region, and the other containing Ab4 as the Fab region. In the protein names, “N” indicates N-terminal, “C” indicates C-terminal, “v” indicates variant, “B” indicates bivalent, and “M” indicates monovalent.

FIG. 4A shows Biacore sensorgrams showing binding between human ST2 and Ab2/IL-2v bispecific molecules.

FIG. 4B shows Biacore sensorgrams showing binding between human ST2 and Ab4/IL-2v bispecific molecules.

FIG. 5A shows Biacore sensorgrams showing binding between human IL2R alpha and Ab2/IL-2v bispecific molecules.

FIG. 5B shows Biacore sensorgrams showing binding between human IL2R alpha and Ab4/IL-2v bispecific molecules.

### DETAILED DESCRIPTION OF THE INVENTION

Regulatory T cells (Tregs) are a class of CD4+CD25+ T cells that suppress the activity of other immune cells, such as CD4+ conventional T cells (Tconv) and CD8+ cells. Tregs are central to immune system homeostasis, maintain tolerance to self-antigens, and modulate the immune response to foreign antigens. Tregs can be robustly activated by Interleukin 2 (IL-2), but IL-2 also activates many other cell types, which can result in significant toxicity. It has become clear that there are subsets of Tregs that can be defined by expression of specific molecular markers and by their roles in different immunological responses. One Treg subset is the ST2+ Treg subset. The defining cell surface marker for ST2+ Tregs is ST2, a component of a cytokine receptor also known interleukin 1 receptor-like 1 protein (IL1RL1), and which is a subunit of the IL-33 receptor. IL-33 is categorized as an “alarmin”, an inflammatory cytokine associated with acute inflammatory responses. ST2+ Tregs are found in tissues such as muscle, visceral adipose, colon, and lung, and possess immunoregulatory and tissue repair functions.

Skeletal muscle is normally devoid of T cells, but following acute muscle injury, large numbers of ST2+ Tregs rapidly migrate into muscle tissue in large numbers. The recruitment of these Tregs into muscle tissue, and production of the growth factor amphiregulin (AREG) has been associated with activation of muscle satellite cells and with tissue repair. Treg deficiency impairs muscle repair after injury, and expansion of Tregs in muscle using IL-2-IL-2R complexes leads to improved outcomes in an animal model of dystrophin-deficient muscular dystrophy. A significant fraction of Tregs that produce AREG are ST2+. ST2+ Tregs also have also been associated with inflamed lung tissue following influenza virus infection, and are associated with lung tissue repair following infection. Treatment of ST2+ Treg with the ligand for the ST2 receptor, IL-33, increases AREG production and tissue repair. Therefore, enhancing the number of ST2+ Tregs or the activity of ST2+ Tregs can treat, or prevent, autoimmune and inflammatory diseases such as inflammatory myopathies, inflammatory muscle diseases, and improve tissue healing after injury or stress.

For example, the role of ST2+ Tregs has been established in animal models of muscle inflammation. One of those animal models is acute muscle injury (Burzyn et al., 2013, Cell 155(6): 1282-1295) in wild type mice, and a second model is the mdx mouse muscular dystrophy model, a model of chronic muscle inflammation caused by genetic deficiency in dystrophin (mdx mice; Villalta et al., 2014, Sci Transl Med 5(258): 258ra142). A role for ST2+ Treg has also been established in a mouse model of inflammatory bowel disease (Schiering et al., 2014, Nature 513(7519):564-568).

By providing compounds and methods that are able to selectively expand ST2+ Tregs numbers and/or enhance ST2+ Treg activity, the present disclosure makes possible new treatments of inflammatory and degenerative diseases. For example, the present disclosure provides a compound with a first moiety that binds to the IL-2 receptor (IL-2R or IL2R) and a second moiety that binds to ST2. IL-2R is a heterotrimeric protein expressed on a variety of different immune cell types, including T cells, NK cells, eosinophils, and monocytes. This broad expression pattern provides a pleiotropic effect on the immune system and a high systemic toxicity of IL-2 treatments, and can make targeting IL-2R+ cells challenging.

IL2-R has three forms, generated by different combinations of three different IL-2R proteins: α (alpha), β (beta), and γ (gamma). These receptor chains assemble to generate the three different receptor forms: (1) the low affinity receptor, IL2Rα, which does not signal; (2) the intermediate affinity receptor (IL2Rβγ), composed of IL2Rβ and IL2Rγ, which is broadly expressed on CD4+ conventional T cells (Tconv), NK cells, eosinophils, and monocytes; and (3) the high affinity receptor (IL2Rαβγ), composed of IL2Rα, IL2Rβ, and IL2Rγ, which is expressed transiently on activated T cells and constitutively on Treg cells. Conventional T cells (Tconv) are those which are activated by antigens and participate in the immune attack. Conventional T cells include helper T cells, cytotoxic T cells, and memory T cells. Mutations in IL-2 can change the binding affinity of IL-2 to different IL-2R receptor forms. Thus, the present disclosure provides compounds that selectively activate and expand Tregs, for example ST2+ Tregs, by comprising a moiety that selectively binds to the high affinity receptor (IL2Rαβγ). An exemplary moiety includes, but is not limited to, an IL-2 variant comprising one or more mutations that modifies the binding relative to wild-type IL-2 so that the IL-2 variant selectively binds to the high affinity receptor (IL2Rαβγ) relative to the intermediate affinity receptor and the low affinity receptor.

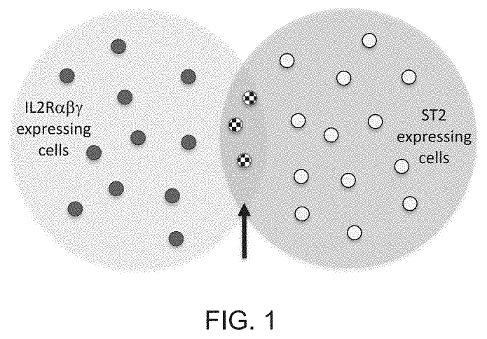

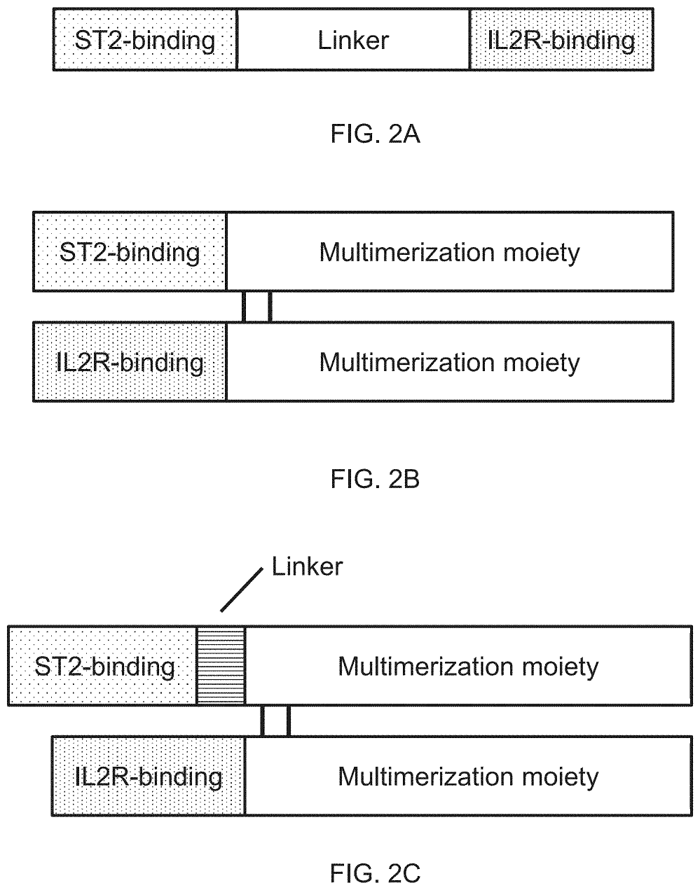

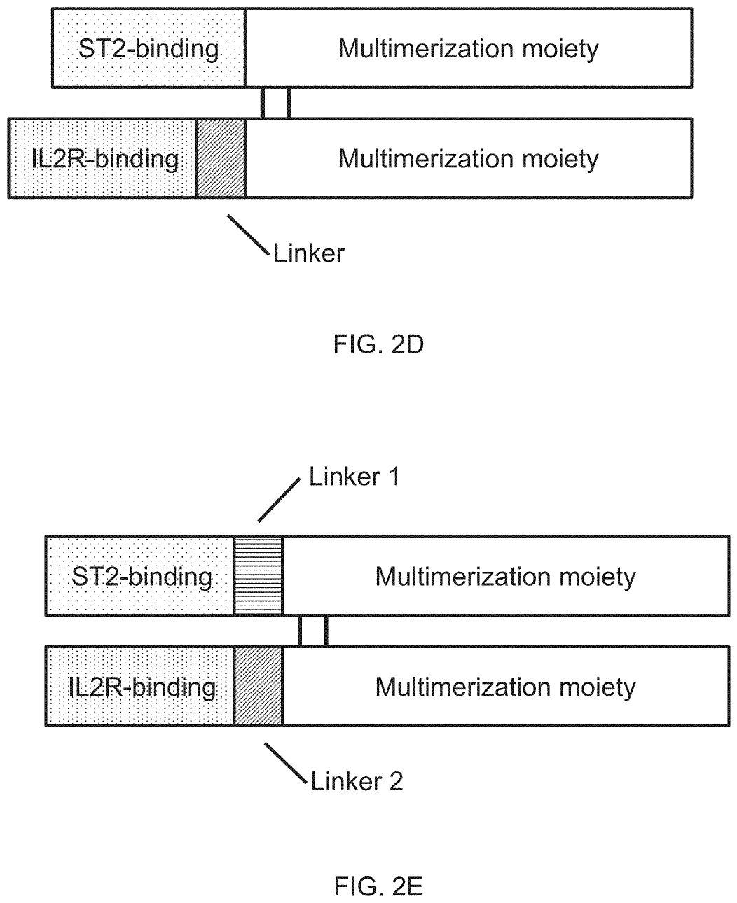

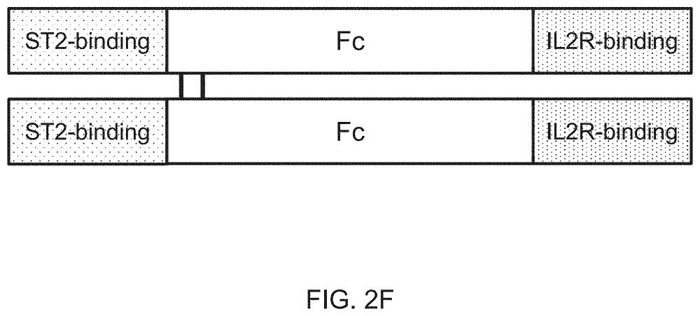

Methods and compositions of the present disclosure relate to a compound comprising a first moiety that binds to the IL-2 receptor and a second moiety that binds to ST2, enabling the compound to be much more selective and potent in its ability to activate and expand ST2+ Tregs. In some embodiments, these compounds target cells that express both the IL-2 receptor or a specific isoform thereof, and ST2. For example, a compound that specifically binds to the IL2Rαβγ isoform can bind to Treg cells expressing ST2. FIG. 1 shows expression domains of IL-2Rαβγ and ST2 in a mixed population of Tregs and an example of an area of overlap where a compound of this disclosure can bind. In some embodiments, the first and second moieties are covalently linked, for example, by an immunoglobulin Fc domain. In some embodiments, the compound also comprises a linker joining the IL-2 receptor-binding moiety and the immunoglobulin Fc domain and/or a linker joining the ST2-binding moiety and the immunoglobulin Fc domain. In some embodiments, the compound can regulate the activities of white blood cells, for example, leukocytes or lymphocytes, that are responsible for immunity. The immunoglobulin Fc domain can increase the in vivo stability of the molecule, and the linker covalently joins a moiety and an Fc domain. By providing moieties that bind to the IL2 receptor and to ST2, the compounds described herein can target cells (for example, T regulatory cells) that express both the IL2 receptor and ST2. In some embodiments, an Fc domain is deficient in its effector functions. An exemplary compound comprises a first moiety that binds to the IL-2 receptor and a second moiety that binds to ST2, wherein the first moiety that binds to the IL-2 receptor is covalently linked to a first effector-deficient Fc domain via a linker and the second moiety that binds to ST2 is covalently linked to a second effector-deficient Fc domain via a linker, and wherein the first effector-deficient Fc domain and the second effector-deficient Fc domain are covalently linked via a disulfide bond. Exemplary compounds are shown in FIGS. 2A-2E, which depict compounds comprising ST2-binding and IL2R-binding moieties, in various combinations with linkers and multimerization domains (which can be, for example, Fc domains). Another example of such a compound is shown in FIG. 2F, wherein an IL2 moiety and an ST2-binding moiety are at the N-terminus and the C-terminus, respectively, of an IgG Fc protein. Such a protein may be in reversed orientation, wherein the ST2-binding moiety is at the N-terminus and the IL2 moiety is at the C-terminus, and may incorporate peptide linkers between one or both of the ST-2 binding moieties and their respective Fc domains, or between one or both of the IL2R binding moieties and their respective Fc domains. An exemplary method for treating a condition, for example, an inflammatory condition such as an inflammatory myopathy, comprises administering to a subject in need thereof a therapeutically-effective amount of a compound as described herein. Exemplary conditions include, but are not limited to, muscular dystrophy and dermatomyositis.

Moieties that Bind the IL-2 Receptor

As described above, the present disclosure provides a compound comprising a first moiety that binds an IL-2 receptor and a second moiety that binds ST2. A moiety that binds an IL-2 receptor can be a polypeptide comprising the full length of wild-type IL-2, shorter, or longer. The IL-2 receptor-binding moiety can have a wild-type IL-2 sequence, as shown in SEQ ID NO: 2: (APTSSSTKKTQLQLEHLLLDLQMILNGINNYKNPKLTRMLTFKFYMPKKATELKHLQCLEE ELKPLEEVLNLAQSKNFHLRPRDLISNINVIVLELKGSETTFMCEYADETATIVEFLNRWITFX QSIISTLT) or a variant of IL-2. IL-2 variants can contain one or more substitutions, deletions, or insertions that deviate from the wild-type IL-2 amino acid sequence. Residues are designated herein by the one letter amino acid code followed by the IL-2 amino acid position, e.g., K35 is the lysine residue at position 35 of the wild-type IL-2 sequence. Substitutions are designated herein by the one letter amino acid code followed by the IL-2 amino acid position followed by the substituting one letter amino acid code, e.g., K35A is a substitution of the lysine residue at position 35 of SEQ ID NO: 2 with an alanine residue.

Compounds herein can exhibit specificity for different IL-2 receptor classes that is similar or dissimilar to the specificity of wild-type IL-2. Compounds herein can exhibit increased stability or biological effect in comparison to wild-type IL-2. For example, a mutation can provide a compound with increased specificity for certain IL-2 receptors in comparison to wild-type IL-2. In some embodiments, a compound selectively binds to the IL2Rαβγ receptor relative to the IL2Rβγ receptor, for example, through its IL-2 binding moiety. In some embodiments, this selective binding is due to one or more mutations in an IL-2 sequence as compared to a wild-type IL-2 sequence. For example, IL-2 N88R is selective for binding to the IL2Rαβγ receptor over the IL2Rβγ receptor. IL-2 can stimulate the proliferation of IL2Rαβγ-expressing PHA-activated T cells as effectively as wildtype IL-2, while exhibiting a 3,000-fold reduced stimulation of the proliferation of IL2Rβγ-expressing NK cells. Other mutations that exhibit increased selectivity for IL2Rαβγ include the substitutions D20H, N88I, N88G, Q126L, and Q126F.

In some embodiments, an IL-2 receptor-binding moiety comprises a mutation that enhances the stability of a compound of the present disclosure. For example, an IL-2 C125S mutation promotes stability by eliminating an unpaired cysteine residue, thereby preventing misfolding of the IL-2 polypeptide. Misfolding can lead to protein aggregation and increase clearance of the polypeptide in vivo. In some embodiments, an IL-2 polypeptide comprises a mutation that creates or removes a glycosylation site. For example the IL-2 mutation T3A removes an O-linked glycosylation site. In some embodiments, an IL-2 variant with the T3A mutation also comprises an N88R mutation and/or a C125S mutation. In some embodiments, an IL-2 variant comprises T3A, N88R, and C125S mutations, as in SEQ ID NO: 3.

In some embodiments, substitutions occur at one or more of positions 3, 20, 88, 125, and 126. In some embodiments, substitutions occur at one, two, three, four, or five of the positions. In some embodiments, an IL-2 variant comprises mutations at positions 88 and 125, for example, N88R and C125S. In some embodiments, an IL-2 receptor-binding moiety comprises the amino acid sequence set forth in SEQ ID NO: 1: APTSSSTKKTQLQLEHLLLDLQMILNGINNYKNPKLTRMLTFKFYMPKKATELKHLQCLEEE LKPLEEVLNLAQSKNFHLRPRDLISRINVIVLELKGSETTFMCEYADETATIVEFLNRWITFSQ SIISTLT. In some embodiments, an IL-2 variant comprises mutations at positions 3, 88 and 125, for example, T3A, N88R and C125S, as in SEQ ID NO: 3: APASSSTKKTQLQLEHLLLDLQMILNGINNYKNPKLTRMLTFKFYMPKKATELKHLQCLEE ELKPLEEVLNLAQSKNFHLRPRDLISRINVIVLELKGSETTFMCEYADETATIVEFLNRWITFS QSIISTLT. In some embodiments, an IL-2 variant comprises 1, 2, 3, 4, 5, 6, 7, 8, 9, 10, 11, 12, 13, 14, 15, 16, 17, 18, 19, or 20 mutations (e.g., substitutions) in comparison to a wild-type IL-2 sequence.

Compounds herein include IL-2 variants comprising an amino acid sequence that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the wild-type IL-2 amino acid sequence (SEQ ID NO: 2). Compounds herein include IL-2 variants comprising an amino acid sequence that is 60%-99% identical to the wild-type IL-2 amino acid sequence, for example, an amino acid sequence that is 80%-99% identical to the wild-type IL-2 amino acid sequence, an amino acid sequence that is 85%-99% identical to the wild-type IL-2 amino acid sequence, an amino acid sequence that is 90%-99% identical to the wild-type IL-2 amino acid sequence, or an amino acid sequence that is 95%-99% identical to the wild-type IL-2 amino acid sequence (SEQ ID NO: 2). Compounds herein include IL-2 variants that comprise an amino acid sequence having an N88R mutation that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the wild-type IL-2 amino acid sequence (SEQ ID NO: 2). Compounds herein include IL-2 variants comprising an amino acid sequence having an N88R mutation that is 60%-99% identical to the wild-type IL-2 amino acid sequence, for example, an amino acid sequence that is 80%-99% identical to the wild-type IL-2 amino acid sequence (SEQ ID NO: 2), an amino acid sequence that is 85%-99% identical to the wild-type IL-2 amino acid sequence (SEQ ID NO: 2), an amino acid sequence that is 90%-99% identical to the wild-type IL-2 amino acid sequence (SEQ ID NO: 2), or an amino acid sequence that is 95%-99% identical to the wild-type IL-2 amino acid sequence (SEQ ID NO: 2).

Embodiments also include IL-2 variants that selectively stimulate Treg cells and comprise an amino acid sequence having N88R and C125S mutations that is at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to the wild-type IL-2 amino acid sequence (SEQ ID NO: 2). Compounds herein include IL-2 variants comprising an amino acid sequence having N88R and C125S mutations that is 60%-99% identical to the wild-type IL-2 amino acid sequence (SEQ ID NO: 2), for example, an amino acid sequence that is 80%-99% identical to the wild-type IL-2 amino acid sequence (SEQ ID NO: 2), an amino acid sequence that is 85%-99% identical to the wild-type IL-2 amino acid sequence (SEQ ID NO: 2), an amino acid sequence that is 90%-99% identical to the wild-type IL-2 amino acid sequence (SEQ ID NO: 2), or an amino acid sequence that is 95%-99% identical to the wild-type IL-2 amino acid sequence (SEQ ID NO: 2).

Compounds also include IL-2 variants that selectively stimulate Treg cells and comprise an amino acid sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the wild-type IL-2 amino acid sequence (SEQ ID NO: 2). Compounds also include IL-2 variants that selectively stimulate Treg cells and comprise an amino acid sequence that is 60%-99% identical to the wild-type IL-2 amino acid sequence (SEQ ID NO: 2), for example, an amino acid sequence that is 80%-99% identical to the wild-type IL-2 amino acid sequence (SEQ ID NO: 2), an amino acid sequence that is 85%-99% identical to the wild-type IL-2 amino acid sequence (SEQ ID NO: 2), an amino acid sequence that is 90%-99% identical to the wild-type IL-2 amino acid sequence (SEQ ID NO: 2), or an amino acid sequence that is 95%-99% identical to the wild-type IL-2 amino acid sequence (SEQ ID NO: 2).

Various methods and software programs can be used to determine the homology between two or more peptides or nucleic acids, such as NCBI BLAST, Clustal W, MAFFT, Clustal Omega, AlignMe, Praline, or another suitable method or algorithm. In some embodiments, percent identity is calculated by FastDB based upon the following parameters: mismatch penalty of 1; gap penalty of 1; gap size penalty of 0.33; and joining penalty of 30.

An example of a useful algorithm is PILEUP. PILEUP creates a multiple sequence alignment from a group of related sequences using progressive, pairwise alignments. The algorithm can also plot a tree showing the clustering relationships used to create the alignment. A non-limiting example of PILEUP parameters includes a default gap weight of 3.00, a default gap length weight of 0.10, and weighted end gaps.

Another example of a useful algorithm is the BLAST algorithm. A non-limiting example of a BLAST program is the WU-BLAST-2 program. WU-BLAST-2 uses several search parameters, most of which are set, for example, to the default values. The adjustable parameters are set, for example, with the following values: overlap span=1, overlap fraction=0.125, word threshold (T)=ll. The HSP S and HSP S2 parameters are dynamic values and are established by the program itself depending upon the composition of the particular sequence and composition of the particular database against which the sequence of interest is being searched. The values can be adjusted to increase sensitivity.

An additional useful algorithm is gapped BLAST. Gapped BLAST uses BLOSUM-62 substitution scores; threshold T parameter set to 9; the two-hit method to trigger ungapped extensions, charges gap lengths of k a cost of 10+k; Xu set to 16, and Xg set to 40 for database search stage and to 67 for the output stage of the algorithms. Gapped alignments are triggered by a score corresponding to, for example, about 22 bits.

An additional useful tool is Clustal, a series of commonly used computer programs for multiple sequence alignment. Recent versions of Clustal include ClustalW, ClustalX and Clustal Omega. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4.

Mutations can be installed at chosen sites or at random. For example, random mutagenesis at a target codon or region can provide mutants to be screened for an activity. Techniques for making substitution mutations at predetermined sites in DNA having a known sequence include, for example, M13 primer mutagenesis and PCR mutagenesis. Screening of the mutants can be accomplished, for example, using assays described herein.

Amino acid substitutions can be of single or multiple residues. Insertions can be, for example, from about 1 to about 20 amino acid residues, or more. Deletions can be, for example, from about 1 to about 20 amino acid residues, or more. Substitutions, deletions, insertions, or any combination thereof can occur in the sample compound.

Moieties that Bind ST2

ST2 (Interleukin 1 receptor-like 1) is a membrane-bound cytokine receptor and a member of the IL-1 receptor family. Human ST2 consists of a 310 amino acid (aa) extracellular domain (ECD) with three Ig-like domains, a 21 aa transmembrane segment, and a 207 aa cytoplasmic domain with an intracellular TIR domain (Tominaga, S. et al., 1992, Biochim. Biophys. Acta 1171:215; and Li, H. et al., 2000, Genomics 67:284). ST2 binds IL-33 and heterodimerizes with the IL-1 receptor accessory protein (1RAcP). In some embodiments, a compound of the present disclosure comprises a binding moiety that binds ST2. Compounds herein can have increased or decreased affinity for ST2 or for the ST2-1RAcP receptor complex. Some compounds may have enhanced affinity for ST2. Other compounds may have reduced affinity for 1RAcP, which would lead to reduced ability to activate the IL-33 receptor.

ST2-binding ligands may include antibodies and antigen-binding antibody fragments with binding affinity toward ST2. As used herein, an “antibody” is a protein that includes at least one complementary determining region that binds to a specific target antigen, e.g. ST2. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. For example, the VH region comprises three CDRs designated as CDRH1, CDRH2, CDRH3, and the VL region comprises three CDRs designated as CDRL1, CDRL2, and CDRL3. The light chains of the immunoglobulin can be of types kappa or lambda. For example, an antibody can be a monoclonal antibody, a modified antibody, a chimeric antibody, a reshaped antibody, or a humanized antibody. The term “monoclonal antibody”, as used herein, refers to a population of antibody molecules that contain only one species of an antigen binding site capable of immunoreacting with a particular epitope. Antibodies can be obtained from commercial sources or produced using known methods. The antibody can be any immunoglobulin type, e.g., IgG, IgM, IgY, IgA1, IgA2, IgD, or IgE. In an embodiment, the antibody can be a human antibody.

Methods and techniques for identifying CDRs within HCVR and LCVR amino acid sequences are known in the art and can be applied to identify CDRs within the specified HCVR and/or LCVR amino acid sequences disclosed herein. Conventional definitions that can be applied to identify the boundaries of CDRs include the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. For example, CDRs can be defined according to the Kabat numbering system (Kabat et al., Sequences of proteins of immunological interest, 5.sup.th Ed., U.S. Department of Health and Human Services, NIH, 1991, and later editions). There are three heavy-chain CDRs and three light-chain CDRs. Here, the terms “CDR” and “CDRs” are used to indicate, depending on the case, one or more, or even all, of the regions containing the majority of the amino acid residues responsible for the antibody's binding affinity for the antigen or epitope it recognizes. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989).

The CDR sequences disclosed herein, for example in Table 2D, indicate the hypervariable regions of the heavy and light chains of the immunoglobulins as defined by IMGT.

The IMGT unique numbering has been defined to compare the variable domains whatever the antigen receptor, the chain type, or the species [Lefranc M.-P., Immunology Today 18, 509 (1997)/Lefranc M.-P., The Immunologist, 7, 132-136 (1999)/Lefranc, M.-P., Pommie, C., Ruiz, M., Giudicelli, V., Foulquier, E., Truong, L., Thouvenin-Contet, V. and Lefranc, Dev. Comp. Immunol., 27, 55-77 (2003)]. In the IMGT unique numbering, the conserved amino acids always have the same position, for instance cystein 23 (1st-CYS), tryptophan 41 (CONSERVED-TRP), hydrophobic amino acid 89, cystein 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT unique numbering is used in 2D graphical representations, designated as IMGT Colliers de Perles [Ruiz, M. and Lefranc, M.-P., Immunogenetics, 53, 857-883 (2002)/Kaas, Q. and Lefranc, M.-P., Current Bioinformatics, 2, 21-30 (2007)], and in 3D structures in IMGT/3D structure-DB [Kaas, Q., Ruiz, M. and Lefranc, M.-P., T cell receptor and MHC structural data. Nucl. Acids. Res., 32, D208-D210 (2004)].

The term “antigen-binding fragment” of an antibody (or simply “antibody fragment”), as used herein, refers to one or more fragments of an antibody that retain the ability to specifically bind to an antigen (e.g., ST1). It has been shown that the antigen-binding function of an antibody can be performed by fragments of a full-length antibody. Antigen-binding antibody fragments (i.e. antibody fragments that specifically bind ST2) suitable for use in the invention include, but are not limited to, a Fab fragment, a F(ab′)2 fragment, a Fd fragment, a Fv fragment, a dAb fragment, single chain Fv (scFv), a dimerized variable region (V region) fragment (diabody), a disulfide-stabilized V region fragment (dsFv), affibodies, antibody mimetics, and one or more isolated complementarity determining regions (CDR) that retain specific binding to ST2. As used herein, an “isolated” CDR is a CDR not in the context of a naturally occurring antibody.

A Fab fragment consists of the light chain variable region (VL), the heavy chain variable region (VH), the light chain constant region (CL) and the heavy chain constant CH1 domain. A F(ab′)2 fragment is a bivalent fragment comprising two linked Fab fragments. An Fd fragment consists of the VH and CH1 domains. The Fv fragment consists of the VL and VH domains of a single antibody. A dAb fragment consists of a VH or a VL domain. A single chain Fv molecule (scFv) consists of a VH domain and a VL domain linked by a peptide linker which allows the two domains to associate to form an antigen binding site. Fv, scFv or diabody molecules may be stabilized by the incorporation of disulphide bridges linking the VH and VL domains. Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding fragments are known in the art (Kontermann and Dubel eds., Antibody Engineering (2001) Springer-Verlag. New York. 790 pp. (ISBN 3-540-41354-5).

Polyclonal antibodies can be prepared by immunizing a suitable subject with a protein of the invention as an immunogen. The antibody titer in the immunized subject can be monitored over time by standard techniques, such as with an enzyme linked immunosorbent assay (ELISA) using immobilized polypeptide. At an appropriate time after immunization, e.g., when the specific antibody titers are highest, antibody-producing cells can be obtained from the subject and used to prepare monoclonal antibodies (mAb) by standard techniques, such as the hybridoma technique originally described by Kohler and Milstein (1975) Nature 256:495-497, the human B cell hybridoma technique (see Kozbor et al., 1983, Immunol. Today 4:72), the EBV-hybridoma technique (see Cole et al., pp. 77-96 In Monoclonal Antibodies and Cancer Therapy, Alan R. Liss, Inc., 1985) or trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest (i.e. ST2), e.g., using a standard ELISA assay.

Alternative to preparing monoclonal antibody-secreting hybridomas, a monoclonal antibody directed against ST2 can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Recombinant antibodies that specifically bind ST2 can also be prepared. Recombinant antibodies include, but are not limited to, chimeric and humanized monoclonal antibodies, comprising both human and non-human portions, single-chain antibodies and multi-specific antibodies. A “fully human” antibody or antibody fragment as defined herein refers to an antibody or antibody fragment in which each portion of the antibody or antibody fragment is of human origin. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Single-chain antibodies have an antigen binding site and consist of a single polypeptide. They can be produced by techniques known in the art, for example using methods described in Ladner et. al U.S. Pat. No. 4,946,778 (which is incorporated herein by reference in its entirety); Bird et al., (1988) Science 242:423-426; Whitlow et al., (1991) Methods in Enzymology 2:1-9; Whitlow et al., (1991) Methods in Enzymology 2:97-105; and Huston et al., (1991) Methods in Enzymology Molecular Design and Modeling: Concepts and Applications 203:46-88.

Humanized antibodies are antibody molecules from non-human species having one or more complementarity determining regions (CDRs) from the non-human species and a framework region from a human immunoglobulin molecule. (See, e.g., Queen, U.S. Pat. No. 5,585,089, which is incorporated herein by reference in its entirety.) Humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

More particularly, humanized antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., ST2. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce IgG, IgA and IgE antibodies. For an overview of this technology for producing human antibodies, see Lonberg and Huszar (1995) Int. Rev. Immunol. 13:65-93). For a detailed discussion of this technology for producing human antibodies and human monoclonal antibodies and protocols for producing such antibodies, see, e.g., U.S. Pat. Nos. 5,625,126; 5,633,425; 5,569,825; 5,661,016; and 5,545,806. In addition, companies can be engaged to provide human antibodies directed against a selected antigen (e.g. ST2) using technology similar to that described above.

Completely human antibodies which recognize ST2 can be generated using a technique referred to as “guided selection.” In this approach a selected non-human monoclonal antibody, e.g., a murine antibody, is used to guide the selection of a completely human antibody recognizing the same epitope (Jespers et al., 1994, Bio/technology 12:899-903).

The ST2 antibodies can be isolated after production (e.g., from the blood or serum of the subject) or synthesis and further purified by well-known techniques. For example, IgG antibodies can be purified using protein A chromatography. Antibodies specific for ST2 can be selected or (e.g., partially purified) or purified by, e.g., affinity chromatography. For example, a recombinantly expressed and purified (or partially purified) ST2 protein is produced, and covalently or non-covalently coupled to a solid support such as, for example, a chromatography column. The column can then be used to affinity purify antibodies specific for ST2 from a sample containing antibodies directed against a large number of different epitopes, thereby generating a substantially purified antibody composition, i.e., one that is substantially free of contaminating antibodies.

Antibodies that bind ST2 are well known in the art. For example, US2017/0002079 describes a range of ST2-binding antibodies (e.g. Ab1, Ab2, Ab3, Ab4 and Ab12-Ab36) directed against human ST2 that were prepared using XENOMOUSE® technology (U.S. Pat. Nos. 6,114,598; 6,162,963; 6,833,268; 7,049,426; 7,064,244, which are incorporated herein by reference in their entirety; Green et al., 1994, Nature Genetics 7:13-21; Mendez et al., 1997, Nature Genetics 15:146-156; Green and Jakobovitis, 1998, J. Ex. Med. 188:483-495, Kellermann and Green, 2002, Current Opinion in Biotechnology, 13:593-597). See, in particular, Example 2 of US2017/0002079, which is incorporated by reference herein in its entirety. Anti-ST2 antibodies directed against human ST2 are also described in WO2012/113813 (e.g. monoclonal antibody ral70) and U.S. Pat. No. 7,087,396 (e.g. monoclonal antibodies 2A5, FB9 and HB12), each of which is incorporated by reference herein in its entirety. For example, U.S. Pat. No. 7,087,396 describes preparation of monoclonal antibodies directed to human ST2 in Example 1. ST2-binding antibodies are also commercially available (e.g. R&D Systems, Inc., Minneapolis, Minn., Cat. Nos. MAB523 and AF523). MAB523 is a monoclonal mouse IgG1 antibody that detects human ST2. AF523 is an antigen affinity-purified polyclonal goat IgG1 that detects human ST2.

In some embodiments, the antibody or antigen-binding fragment thereof that specifically binds ST2 comprises a heavy chain and a light chain. In some embodiments, the heavy chain comprises the amino acid sequence of SEQ ID NO: 16, and the light chain comprises the amino acid sequence of SEQ ID NO: 19 (Ab2). In some embodiments, the heavy chain comprises the amino acid sequence of SEQ ID NO: 17, and the light chain comprises the amino acid sequence of SEQ ID NO: 20 (Ab4). In some embodiments, the heavy chain comprises an amino acid sequence having at least 90%, at least 95%, at least 96%, at least 97%, at least 98%, or at least 99% sequence identity to the amino acid sequence of SEQ ID NO: 16. In some embodiments, the heavy chain comprises an amino acid sequence having at least 90%, at least 95%, at least 96%, at least 97%, at least 98%, or at least 99% sequence identity to the amino acid sequence of SEQ ID NO: 17. In some embodiments, the light chain comprises an amino acid sequence having at least 90%, at least 95%, at least 96%, at least 97%, at least 98%, or at least 99% sequence identity to the amino acid sequence of SEQ ID NO: 19. In some embodiments, the light chain comprises an amino acid sequence having at least 90%, at least 95%, at least 96%, at least 97%, at least 98%, or at least 99% sequence identity to the amino acid sequence of SEQ ID NO: 20.

In some embodiments, the antibody or antigen-binding fragment thereof that specifically binds ST2 comprises a combination of a heavy chain and a light chain as shown in Table 1 below. Table 1 lists the human ST2 IgG1 antibodies that were identified through screening of a phage display human antibody library, as described in Example 3. The IgG1 heavy chain and light chain for each of the 109 ST2 antibodies identified in the screen are provided. For example, in some embodiments, the antibody or antigen-binding fragment thereof that specifically binds ST2 comprises a heavy chain comprising the amino acid sequence of SEQ ID NO: 23 and a light chain comprising the amino acid sequence of SEQ ID NO: 241 (e.g. ST2 Antibody 1). In some embodiments, the antibody or antigen-binding fragment thereof that specifically binds ST2 comprises a heavy chain encoded by the nucleic acid sequence of SEQ ID NO: 22 and a light chain encoded by the nucleic acid sequence of SEQ ID NO: 240 (e.g. ST2 Antibody 1). In some embodiments, the antibody or antigen-binding fragment thereof that specifically binds ST2 comprises a heavy chain comprising an amino acid sequence having at least 90%, at least 95%, at least 96%, at least 97%, at least 98%, or at least 99% sequence identity to an IgG1 heavy chain amino acid sequence listed in Table 1. In some embodiments, the antibody or antigen-binding fragment thereof that specifically binds ST2 comprises a light chain comprising an amino acid sequence having at least 90%, at least 95%, at least 96%, at least 97%, at least 98%, or at least 99% sequence identity to a light chain amino acid sequence listed in Table 1. In some embodiments, the antibody or antigen-binding fragment thereof that specifically binds ST2 comprises a heavy chain encoded by a nucleic acid sequence having at least 90%, at least 95%, at least 96%, at least 97%, at least 98%, or at least 99% sequence identity to an IgG1 heavy chain nucleic acid sequence listed in Table 1. In some embodiments, the antibody or antigen-binding fragment thereof that specifically binds ST2 comprises a light chain encoded by a nucleic acid sequence having at least 90%, at least 95%, at least 96%, at least 97%, at least 98%, or at least 99% sequence identity to a light chain nucleic acid sequence listed in Table 1.

In some embodiments, the antibody or antigen-binding fragment thereof that specifically binds ST2 comprises or consists of a human ST2 IgG1 antibody listed in Table 1. In some embodiments, the antibody or antigen-binding fragment thereof that specifically binds ST2 comprises or consists of a human ST2 IgG1 antibody having at least 90%, at least 95%, at least 96%, at least 97%, at least 98%, or at least 99% sequence identity to an ST2 antibody listed in Table 1.

The human ST2 IgG1 antibody heavy chains in Table 1 consist of the signal peptide (amino acids 1-24), the heavy chain variable region, the CH1 domain, and the Fc domain. For example, in SEQ ID NO: 23, amino acids 1-24 are the human IgG1 heavy chain signal peptide, amino acids 25-147 are the heavy chain variable region, amino acids 148-251 are the human IgG1 heavy chain constant domain CH1, and amino acids 252-476 are the human IgG1 Fc domain. The human ST2 IgG1 antibody lights chains in Table 1 consist of the signal peptide (amino acids 1-20), the light chain variable region, and the light chain constant domain. For example, in SEQ ID NO: 241, amino acids 1-20 are the human IgG1 light chain signal peptide, amino acids 21-132 are the light chain variable region, and amino acids 133-239 are the human IgG1 light chain constant domain.

TABLE 1
Human ST2 IgG1 antibody heavy chains and light chains.

| Human | IgG1 Heavy | Light Chain | IgG1 Heavy | Light Chain |
|---|---|---|---|---|
| ST2 IgG1 | Chain Amino | (Kappa) Amino | Chain Nucleic | (Kappa) Nucleic |
| Antibody | Acid Sequence | Acid Sequence | Acid Sequence | AcidSequence |
| 1 | SEQ ID NO: 23 | SEQ ID NO: 241 | SEQ ID NO: 22 | SEQ ID NO: 240 |
| 2 | SEQ ID NO: 25 | SEQ ID NO: 243 | SEQ ID NO: 24 | SEQ ID NO: 242 |
| 3 | SEQ ID NO: 27 | SEQ ID NO: 245 | SEQ ID NO: 26 | SEQ ID NO: 244 |
| 4 | SEQ ID NO: 29 | SEQ ID NO: 247 | SEQ ID NO: 28 | SEQ ID NO: 246 |
| 5 | SEQ ID NO: 31 | SEQ ID NO: 249 | SEQ ID NO: 30 | SEQ ID NO: 248 |
| 6 | SEQ ID NO: 33 | SEQ ID NO: 251 | SEQ ID NO: 32 | SEQ ID NO: 250 |
| 7 | SEQ ID NO: 35 | SEQ ID NO: 253 | SEQ ID NO: 34 | SEQ ID NO: 252 |
| 8 | SEQ ID NO: 37 | SEQ ID NO: 255 | SEQ ID NO: 36 | SEQ ID NO: 254 |
| 9 | SEQ ID NO: 39 | SEQ ID NO: 257 | SEQ ID NO: 38 | SEQ ID NO: 256 |
| 10 | SEQ ID NO: 41 | SEQ ID NO: 259 | SEQ ID NO: 40 | SEQ ID NO: 258 |
| 11 | SEQ ID NO: 43 | SEQ ID NO: 261 | SEQ ID NO: 42 | SEQ ID NO: 260 |
| 12 | SEQ ID NO: 45 | SEQ ID NO: 263 | SEQ ID NO: 44 | SEQ ID NO: 262 |
| 13 | SEQ ID NO: 47 | SEQ ID NO: 265 | SEQ ID NO: 46 | SEQ ID NO: 264 |
| 14 | SEQ ID NO: 49 | SEQ ID NO: 267 | SEQ ID NO: 48 | SEQ ID NO: 266 |
| 15 | SEQ ID NO: 51 | SEQ ID NO: 269 | SEQ ID NO: 50 | SEQ ID NO: 268 |
| 16 | SEQ ID NO: 53 | SEQ ID NO: 271 | SEQ ID NO: 52 | SEQ ID NO: 270 |
| 17 | SEQ ID NO: 55 | SEQ ID NO: 273 | SEQ ID NO: 54 | SEQ ID NO: 272 |
| 18 | SEQ ID NO: 57 | SEQ ID NO: 275 | SEQ ID NO: 56 | SEQ ID NO: 274 |
| 19 | SEQ ID NO: 59 | SEQ ID NO: 277 | SEQ ID NO: 58 | SEQ ID NO: 276 |
| 20 | SEQ ID NO: 61 | SEQ ID NO: 279 | SEQ ID NO: 60 | SEQ ID NO: 278 |
| 21 | SEQ ID NO: 63 | SEQ ID NO: 281 | SEQ ID NO: 62 | SEQ ID NO: 280 |
| 22 | SEQ ID NO: 65 | SEQ ID NO: 283 | SEQ ID NO: 64 | SEQ ID NO: 282 |
| 23 | SEQ ID NO: 67 | SEQ ID NO: 285 | SEQ ID NO: 66 | SEQ ID NO: 284 |
| 24 | SEQ ID NO: 69 | SEQ ID NO: 287 | SEQ ID NO: 68 | SEQ ID NO: 286 |
| 25 | SEQ ID NO: 71 | SEQ ID NO: 289 | SEQ ID NO: 70 | SEQ ID NO: 288 |
| 26 | SEQ ID NO: 73 | SEQ ID NO: 291 | SEQ ID NO: 72 | SEQ ID NO: 290 |
| 27 | SEQ ID NO: 75 | SEQ ID NO: 293 | SEQ ID NO: 74 | SEQ ID NO: 292 |
| 28 | SEQ ID NO: 77 | SEQ ID NO: 295 | SEQ ID NO: 76 | SEQ ID NO: 294 |
| 29 | SEQ ID NO: 79 | SEQ ID NO: 297 | SEQ ID NO: 78 | SEQ ID NO: 296 |
| 30 | SEQ ID NO: 81 | SEQ ID NO: 299 | SEQ ID NO: 80 | SEQ ID NO: 298 |
| 31 | SEQ ID NO: 83 | SEQ ID NO: 301 | SEQ ID NO: 82 | SEQ ID NO: 300 |
| 32 | SEQ ID NO: 85 | SEQ ID NO: 303 | SEQ ID NO: 84 | SEQ ID NO: 302 |
| 33 | SEQ ID NO: 87 | SEQ ID NO: 305 | SEQ ID NO: 86 | SEQ ID NO: 304 |
| 34 | SEQ ID NO: 89 | SEQ ID NO: 307 | SEQ ID NO: 88 | SEQ ID NO: 306 |
| 35 | SEQ ID NO: 91 | SEQ ID NO: 309 | SEQ ID NO: 90 | SEQ ID NO: 308 |
| 36 | SEQ ID NO: 93 | SEQ ID NO: 311 | SEQ ID NO: 92 | SEQ ID NO: 310 |
| 37 | SEQ ID NO: 95 | SEQ ID NO: 313 | SEQ ID NO: 94 | SEQ ID NO: 312 |
| 38 | SEQ ID NO: 97 | SEQ ID NO: 315 | SEQ ID NO: 96 | SEQ ID NO: 314 |
| 39 | SEQ ID NO: 99 | SEQ ID NO: 317 | SEQ ID NO: 98 | SEQ ID NO: 316 |
| 40 | SEQ ID NO: 101 | SEQ ID NO: 319 | SEQ ID NO: 100 | SEQ ID NO: 318 |
| 41 | SEQ ID NO: 103 | SEQ ID NO: 321 | SEQ ID NO: 102 | SEQ ID NO: 320 |
| 42 | SEQ ID NO: 105 | SEQ ID NO: 323 | SEQ ID NO: 104 | SEQ ID NO: 322 |
| 43 | SEQ ID NO: 107 | SEQ ID NO: 325 | SEQ ID NO: 106 | SEQ ID NO: 324 |
| 44 | SEQ ID NO: 109 | SEQ ID NO: 327 | SEQ ID NO: 108 | SEQ ID NO: 326 |
| 45 | SEQ ID NO: 111 | SEQ ID NO: 329 | SEQ ID NO: 110 | SEQ ID NO: 328 |
| 46 | SEQ ID NO: 113 | SEQ ID NO: 331 | SEQ ID NO: 112 | SEQ ID NO: 330 |
| 47 | SEQ ID NO: 115 | SEQ ID NO: 333 | SEQ ID NO: 114 | SEQ ID NO: 332 |
| 48 | SEQ ID NO: 117 | SEQ ID NO: 335 | SEQ ID NO: 116 | SEQ ID NO: 334 |
| 49 | SEQ ID NO: 119 | SEQ ID NO: 337 | SEQ ID NO: 118 | SEQ ID NO: 336 |
| 50 | SEQ ID NO: 121 | SEQ ID NO: 339 | SEQ ID NO: 120 | SEQ ID NO: 338 |
| 51 | SEQ ID NO: 123 | SEQ ID NO: 341 | SEQ ID NO: 122 | SEQ ID NO: 340 |
| 52 | SEQ ID NO: 125 | SEQ ID NO: 343 | SEQ ID NO: 124 | SEQ ID NO: 342 |
| 53 | SEQ ID NO: 127 | SEQ ID NO: 345 | SEQ ID NO: 126 | SEQ ID NO: 344 |
| 54 | SEQ ID NO: 129 | SEQ ID NO: 347 | SEQ ID NO: 128 | SEQ ID NO: 346 |
| 55 | SEQ ID NO: 131 | SEQ ID NO: 349 | SEQ ID NO: 130 | SEQ ID NO: 348 |
| 56 | SEQ ID NO: 133 | SEQ ID NO: 351 | SEQ ID NO: 132 | SEQ ID NO: 350 |
| 57 | SEQ ID NO: 135 | SEQ ID NO: 353 | SEQ ID NO: 134 | SEQ ID NO: 352 |
| 58 | SEQ ID NO: 137 | SEQ ID NO: 355 | SEQ ID NO: 136 | SEQ ID NO: 354 |
| 59 | SEQ ID NO: 139 | SEQ ID NO: 357 | SEQ ID NO: 138 | SEQ ID NO: 356 |
| 60 | SEQ ID NO: 141 | SEQ ID NO: 359 | SEQ ID NO: 140 | SEQ ID NO: 358 |
| 61 | SEQ ID NO: 143 | SEQ ID NO: 361 | SEQ ID NO: 142 | SEQ ID NO: 360 |
| 62 | SEQ ID NO: 145 | SEQ ID NO: 363 | SEQ ID NO: 144 | SEQ ID NO: 362 |
| 63 | SEQ ID NO: 147 | SEQ ID NO: 365 | SEQ ID NO: 146 | SEQ ID NO: 364 |
| 64 | SEQ ID NO: 149 | SEQ ID NO: 367 | SEQ ID NO: 148 | SEQ ID NO: 366 |
| 65 | SEQ ID NO: 151 | SEQ ID NO: 369 | SEQ ID NO: 150 | SEQ ID NO: 368 |
| 66 | SEQ ID NO: 153 | SEQ ID NO: 371 | SEQ ID NO: 152 | SEQ ID NO: 370 |
| 67 | SEQ ID NO: 155 | SEQ ID NO: 373 | SEQ ID NO: 154 | SEQ ID NO: 372 |
| 68 | SEQ ID NO: 157 | SEQ ID NO: 375 | SEQ ID NO: 156 | SEQ ID NO: 374 |
| 69 | SEQ ID NO: 159 | SEQ ID NO: 377 | SEQ ID NO: 158 | SEQ ID NO: 376 |
| 70 | SEQ ID NO: 161 | SEQ ID NO: 379 | SEQ ID NO: 160 | SEQ ID NO: 378 |
| 71 | SEQ ID NO: 163 | SEQ ID NO: 381 | SEQ ID NO: 162 | SEQ ID NO: 380 |
| 72 | SEQ ID NO: 165 | SEQ ID NO: 383 | SEQ ID NO: 164 | SEQ ID NO: 382 |
| 73 | SEQ ID NO: 167 | SEQ ID NO: 385 | SEQ ID NO: 166 | SEQ ID NO: 384 |
| 74 | SEQ ID NO: 169 | SEQ ID NO: 387 | SEQ ID NO: 168 | SEQ ID NO: 386 |
| 75 | SEQ ID NO: 171 | SEQ ID NO: 389 | SEQ ID NO: 170 | SEQ ID NO: 388 |
| 76 | SEQ ID NO: 173 | SEQ ID NO: 391 | SEQ ID NO: 172 | SEQ ID NO: 390 |
| 77 | SEQ ID NO: 175 | SEQ ID NO: 393 | SEQ ID NO: 174 | SEQ ID NO: 392 |
| 78 | SEQ ID NO: 177 | SEQ ID NO: 395 | SEQ ID NO: 176 | SEQ ID NO: 394 |
| 79 | SEQ ID NO: 179 | SEQ ID NO: 397 | SEQ ID NO: 178 | SEQ ID NO: 396 |
| 80 | SEQ ID NO: 181 | SEQ ID NO: 399 | SEQ ID NO: 180 | SEQ ID NO: 398 |
| 81 | SEQ ID NO: 183 | SEQ ID NO: 401 | SEQ ID NO: 182 | SEQ ID NO: 400 |
| 82 | SEQ ID NO: 185 | SEQ ID NO: 403 | SEQ ID NO: 184 | SEQ ID NO: 402 |
| 83 | SEQ ID NO: 187 | SEQ ID NO: 405 | SEQ ID NO: 186 | SEQ ID NO: 404 |
| 84 | SEQ ID NO: 189 | SEQ ID NO: 407 | SEQ ID NO: 188 | SEQ ID NO: 406 |
| 85 | SEQ ID NO: 191 | SEQ ID NO: 409 | SEQ ID NO: 190 | SEQ ID NO: 408 |
| 86 | SEQ ID NO: 193 | SEQ ID NO: 411 | SEQ ID NO: 192 | SEQ ID NO: 410 |
| 87 | SEQ ID NO: 195 | SEQ ID NO: 413 | SEQ ID NO: 194 | SEQ ID NO: 412 |
| 88 | SEQ ID NO: 197 | SEQ ID NO: 415 | SEQ ID NO: 196 | SEQ ID NO: 414 |
| 89 | SEQ ID NO: 199 | SEQ ID NO: 417 | SEQ ID NO: 198 | SEQ ID NO: 416 |
| 90 | SEQ ID NO: 201 | SEQ ID NO: 419 | SEQ ID NO: 200 | SEQ ID NO: 418 |
| 91 | SEQ ID NO: 203 | SEQ ID NO: 421 | SEQ ID NO: 202 | SEQ ID NO: 420 |
| 92 | SEQ ID NO: 205 | SEQ ID NO: 423 | SEQ ID NO: 204 | SEQ ID NO: 422 |
| 93 | SEQ ID NO: 207 | SEQ ID NO: 425 | SEQ ID NO: 206 | SEQ ID NO: 424 |
| 94 | SEQ ID NO: 209 | SEQ ID NO: 427 | SEQ ID NO: 208 | SEQ ID NO: 426 |
| 95 | SEQ ID NO: 211 | SEQ ID NO: 429 | SEQ ID NO: 210 | SEQ ID NO: 428 |
| 96 | SEQ ID NO: 213 | SEQ ID NO: 431 | SEQ ID NO: 212 | SEQ ID NO: 430 |
| 97 | SEQ ID NO: 215 | SEQ ID NO: 433 | SEQ ID NO: 214 | SEQ ID NO: 432 |
| 98 | SEQ ID NO: 217 | SEQ ID NO: 435 | SEQ ID NO: 216 | SEQ ID NO: 434 |
| 99 | SEQ ID NO: 219 | SEQ ID NO: 437 | SEQ ID NO: 218 | SEQ ID NO: 436 |
| 100 | SEQ ID NO: 221 | SEQ ID NO: 439 | SEQ ID NO: 220 | SEQ ID NO: 438 |
| 101 | SEQ ID NO: 223 | SEQ ID NO: 441 | SEQ ID NO: 222 | SEQ ID NO: 440 |
| 102 | SEQ ID NO: 225 | SEQ ID NO: 443 | SEQ ID NO: 224 | SEQ ID NO: 442 |
| 103 | SEQ ID NO: 227 | SEQ ID NO: 445 | SEQ ID NO: 226 | SEQ ID NO: 444 |
| 104 | SEQ ID NO: 229 | SEQ ID NO: 447 | SEQ ID NO: 228 | SEQ ID NO: 446 |
| 105 | SEQ ID NO: 231 | SEQ ID NO: 449 | SEQ ID NO: 230 | SEQ ID NO: 448 |
| 106 | SEQ ID NO: 233 | SEQ ID NO: 451 | SEQ ID NO: 232 | SEQ ID NO: 450 |
| 107 | SEQ ID NO: 235 | SEQ ID NO: 453 | SEQ ID NO: 234 | SEQ ID NO: 452 |
| 108 | SEQ ID NO: 237 | SEQ ID NO: 455 | SEQ ID NO: 236 | SEQ ID NO: 454 |
| 109 | SEQ ID NO: 239 | SEQ ID NO: 457 | SEQ ID NO: 238 | SEQ ID NO: 456 |

In some embodiments, the antibody or antigen-binding fragment thereof that specifically binds ST2 comprises or consists of a fragment of a human ST2 IgG1 antibody listed in Table 1 that specifically binds ST2. For example, in some embodiments, the antibody or antigen-binding fragment thereof that specifically binds ST2 comprises or consists of an IgG1 heavy chain fragment listed in Table 2A or 2B. Table 2A lists fragments of the IgG1 heavy chain amino acid sequences listed in Table 1 in which the Fc region has been removed, i.e. the fragment consists of the heavy chain variable region and the C_{H}1 heavy chain constant region. Table 2B lists the heavy chain variable region of each ST2 antibody. In some embodiments, the antibody or antigen-binding fragment thereof that specifically binds ST2 comprises an IgG1 heavy chain fragment having at least 85% sequence identity to an IgG1 heavy chain fragment listed in Table 2A or 2B. In some embodiments, the antibody or antigen-binding fragment thereof that specifically binds ST2 comprises an IgG1 heavy chain fragment having at least 90% sequence identity to an IgG1 heavy chain fragment listed in Table 2A or 2B. In some embodiments, the antibody or antigen-binding fragment thereof that specifically binds ST2 comprises an IgG1 heavy chain fragment having at least 95% sequence identity to an IgG1 heavy chain fragment listed in Table 2A or 2B. In some embodiments, the antibody or antigen-binding fragment thereof that specifically binds ST2 comprises an IgG1 heavy chain fragment having at least 96% sequence identity to an IgG1 heavy chain fragment listed in Table 2A or 2B. In some embodiments, the antibody or antigen-binding fragment thereof that specifically binds ST2 comprises an IgG1 heavy chain fragment having at least 97% sequence identity to an IgG1 heavy chain fragment listed in Table 2A or 2B. In some embodiments, the antibody or antigen-binding fragment thereof that specifically binds ST2 comprises an IgG1 heavy chain fragment having at least 98% sequence identity to an IgG1 heavy chain fragment listed in Table 2A or 2B. In some embodiments, the antibody or antigen-binding fragment thereof that specifically binds ST2 comprises an IgG1 heavy chain fragment having at least 99% sequence identity to an IgG1 heavy chain fragment listed in Table 2A or 2B.

In some embodiments, the antibody or antigen-binding fragment thereof that specifically binds ST2 comprises or consists of an IgG1 light chain variable region listed in Table 2C. In some embodiments, the antibody or antigen-binding fragment thereof that specifically binds ST2 comprises or consists of a heavy chain variable region listed in Table 2B and the corresponding light chain variable region from the same antibody listed in Table 2C. For example, in some embodiments, the antibody or antigen-binding fragment thereof that specifically binds ST2 comprises or consists of the heavy chain variable region of Antibody 1 (i.e. amino acids 1-147 of SEQ ID NO: 23) and the light chain variable region of Antibody 1 (i.e. amino acids 1-132 of SEQ ID NO: 241). In some embodiments, the antibody or antigen-binding fragment thereof that specifically binds ST2 comprises or consists of a heavy chain fragment (heavy chain variable region +C_{H}1) listed in Table 2A and the corresponding light chain from the same antibody listed in Table 2C. For example, in some embodiments, the antibody or antigen-binding fragment thereof that specifically binds ST2 comprises or consists of amino acids 1-251 of SEQ ID NO: 23 (Antibody 1) and the light chain of Antibody 1 (i.e. SEQ ID NO: 24).

In some embodiments, the antibody or antigen-binding fragment thereof that specifically binds ST2 comprises an IgG1 light chain variable region having at least 85% sequence identity to an IgG1 light chain variable region listed in Table 2C. In some embodiments, the antibody or antigen-binding fragment thereof that specifically binds ST2 comprises an IgG1 light chain variable region having at least 90% sequence identity to an IgG1 light chain variable region listed in Table 2C. In some embodiments, the antibody or antigen-binding fragment thereof that specifically binds ST2 comprises an IgG1 light chain variable region having at least 95% sequence identity to an IgG1 light chain variable region listed in Table 2C. In some embodiments, the antibody or antigen-binding fragment thereof that specifically binds ST2 comprises an IgG1 light chain variable region having at least 96% sequence identity to an IgG1 light chain variable region listed in Table 2C. In some embodiments, the antibody or antigen-binding fragment thereof that specifically binds ST2 comprises an IgG1 light chain variable region having at least 97% sequence identity to an IgG1 light chain variable region listed in Table 2C. In some embodiments, the antibody or antigen-binding fragment thereof that specifically binds ST2 comprises an IgG1 light chain variable region having at least 98% sequence identity to an IgG1 light chain variable region listed in Table 2C. In some embodiments, the antibody or antigen-binding fragment thereof that specifically binds ST2 comprises an IgG1 light chain variable region having at least 99% sequence identity to an IgG1 light chain variable region listed in Table 2C.

TABLE 2A
ST2 antibody IgG1 heavy chain fragments. Each fragment consists
of the heavy chain variable region and the heavy chain constant region
C_{H}1. The fragments do not contain the Fc region. The amino acid
numbers refer to the amino acid positions in the listed sequence.
For example, the IgG1 heavy chain fragment of Antibody 1 consists
of amino acid positions 25-251 of SEQ ID NO: 23.

| Human | IgG1 Heavy | Amino Acids in |
|---|---|---|
| ST2 IgG1 | Chain Amino | IgG1 Heavy Chain |
| Antibody | Acid Sequence | Fragment |
| 1 | SEQ ID NO: 23 | 25-251 |
| 2 | SEQ ID NO: 25 | 25-250 |
| 3 | SEQ ID NO: 27 | 25-249 |
| 4 | SEQ ID NO: 29 | 25-252 |
| 5 | SEQ ID NO: 31 | 25-245 |
| 6 | SEQ ID NO: 33 | 25-247 |
| 7 | SEQ ID NO: 35 | 25-254 |
| 8 | SEQ ID NO: 37 | 25-252 |
| 9 | SEQ ID NO: 39 | 25-250 |
| 10 | SEQ ID NO: 41 | 25-249 |
| 11 | SEQ ID NO: 43 | 25-248 |
| 12 | SEQ ID NO: 45 | 25-255 |
| 13 | SEQ ID NO: 47 | 25-245 |
| 14 | SEQ ID NO: 49 | 25-244 |
| 15 | SEQ ID NO: 51 | 25-249 |
| 16 | SEQ ID NO: 53 | 25-256 |
| 17 | SEQ ID NO: 55 | 25-246 |
| 18 | SEQ ID NO: 57 | 25-251 |
| 19 | SEQ ID NO: 59 | 25-245 |
| 20 | SEQ ID NO: 61 | 25-252 |
| 21 | SEQ ID NO: 63 | 25-246 |
| 22 | SEQ ID NO: 65 | 25-249 |
| 23 | SEQ ID NO: 67 | 25-244 |
| 24 | SEQ ID NO: 69 | 25-249 |
| 25 | SEQ ID NO: 71 | 25-244 |
| 26 | SEQ ID NO: 73 | 25-244 |
| 27 | SEQ ID NO: 75 | 25-249 |
| 28 | SEQ ID NO: 77 | 25-247 |
| 29 | SEQ ID NO: 79 | 25-247 |
| 30 | SEQ ID NO: 81 | 25-255 |
| 31 | SEQ ID NO: 83 | 25-246 |
| 32 | SEQ ID NO: 85 | 25-248 |
| 33 | SEQ ID NO: 87 | 25-252 |
| 34 | SEQ ID NO: 89 | 25-248 |
| 35 | SEQ ID NO: 91 | 25-244 |
| 36 | SEQ ID NO: 93 | 25-247 |
| 37 | SEQ ID NO: 95 | 25-254 |
| 38 | SEQ ID NO: 97 | 25-251 |
| 39 | SEQ ID NO: 99 | 25-245 |
| 40 | SEQ ID NO: 101 | 25-257 |
| 41 | SEQ ID NO: 103 | 25-256 |
| 42 | SEQ ID NO: 105 | 25-249 |
| 43 | SEQ ID NO: 107 | 25-248 |
| 44 | SEQ ID NO: 109 | 25-250 |
| 45 | SEQ ID NO: 111 | 25-244 |
| 46 | SEQ ID NO: 113 | 25-247 |
| 47 | SEQ ID NO: 115 | 25-248 |
| 48 | SEQ ID NO: 117 | 25-245 |
| 49 | SEQ ID NO: 119 | 25-253 |
| 50 | SEQ ID NO: 121 | 25-249 |
| 51 | SEQ ID NO: 123 | 25-253 |
| 52 | SEQ ID NO: 125 | 25-253 |
| 53 | SEQ ID NO: 127 | 25-251 |
| 54 | SEQ ID NO: 129 | 25-245 |
| 55 | SEQ ID NO: 131 | 25-249 |
| 56 | SEQ ID NO: 133 | 25-250 |
| 57 | SEQ ID NO: 135 | 25-256 |
| 58 | SEQ ID NO: 137 | 25-250 |
| 59 | SEQ ID NO: 139 | 25-247 |
| 60 | SEQ ID NO: 141 | 25-253 |
| 61 | SEQ ID NO: 143 | 25-249 |
| 62 | SEQ ID NO: 145 | 25-246 |
| 63 | SEQ ID NO: 147 | 25-251 |
| 64 | SEQ ID NO: 149 | 25-245 |
| 65 | SEQ ID NO: 151 | 25-244 |
| 66 | SEQ ID NO: 153 | 25-249 |
| 67 | SEQ ID NO: 155 | 25-257 |
| 68 | SEQ ID NO: 157 | 25-250 |
| 69 | SEQ ID NO: 159 | 25-253 |
| 70 | SEQ ID NO: 161 | 25-245 |
| 71 | SEQ ID NO: 163 | 25-260 |
| 72 | SEQ ID NO: 165 | 25-245 |
| 73 | SEQ ID NO: 167 | 25-244 |
| 74 | SEQ ID NO: 169 | 25-244 |
| 75 | SEQ ID NO: 171 | 25-245 |
| 76 | SEQ ID NO: 173 | 25-244 |
| 77 | SEQ ID NO: 175 | 25-248 |
| 78 | SEQ ID NO: 177 | 25-246 |
| 79 | SEQ ID NO: 179 | 25-249 |
| 80 | SEQ ID NO: 181 | 25-249 |
| 81 | SEQ ID NO: 183 | 25-247 |
| 82 | SEQ ID NO: 185 | 25-251 |
| 83 | SEQ ID NO: 187 | 25-247 |
| 84 | SEQ ID NO: 189 | 25-249 |
| 85 | SEQ ID NO: 191 | 25-248 |
| 86 | SEQ ID NO: 193 | 25-246 |
| 87 | SEQ ID NO: 195 | 25-250 |
| 88 | SEQ ID NO: 197 | 25-245 |
| 89 | SEQ ID NO: 199 | 25-250 |
| 90 | SEQ ID NO: 201 | 25-246 |
| 91 | SEQ ID NO: 203 | 25-243 |
| 92 | SEQ ID NO: 205 | 25-248 |
| 93 | SEQ ID NO: 207 | 25-250 |
| 94 | SEQ ID NO: 209 | 25-249 |
| 95 | SEQ ID NO: 211 | 25-255 |
| 96 | SEQ ID NO: 213 | 25-245 |
| 97 | SEQ ID NO: 215 | 25-244 |
| 98 | SEQ ID NO: 217 | 25-250 |
| 99 | SEQ ID NO: 219 | 25-246 |
| 100 | SEQ ID NO: 221 | 25-247 |
| 101 | SEQ ID NO: 223 | 25-254 |
| 102 | SEQ ID NO: 225 | 25-248 |
| 103 | SEQ ID NO: 227 | 25-249 |
| 104 | SEQ ID NO: 229 | 25-253 |
| 105 | SEQ ID NO: 231 | 25-251 |
| 106 | SEQ ID NO: 233 | 25-244 |
| 107 | SEQ ID NO: 235 | 25-245 |
| 108 | SEQ ID NO: 237 | 25-244 |
| 109 | SEQ ID NO: 239 | 25-254 |

TABLE 2B
ST2 antibody IgG1 heavy chain variable regions. The amino acid
numbers refer to the amino acid positions in the listed sequence.
For example, the IgG1 heavy chain variable region of Antibody
1 consists of amino acid positions 25-147 of SEQ ID NO: 23.

| Human | IgG1 Heavy | Amino Acids in |
|---|---|---|
| ST2 IgG1 | Chain Amino | IgG1 Heavy Chain |
| Antibody | Acid Sequence | Variable Region |
| 1 | SEQ ID NO: 23 | 25-147 |
| 2 | SEQ ID NO: 25 | 25-147 |
| 3 | SEQ ID NO: 27 | 25-145 |
| 4 | SEQ ID NO: 29 | 25-148 |
| 5 | SEQ ID NO: 31 | 25-141 |
| 6 | SEQ ID NO: 33 | 25-143 |
| 7 | SEQ ID NO: 35 | 25-150 |
| 8 | SEQ ID NO: 37 | 25-148 |
| 9 | SEQ ID NO: 39 | 25-146 |
| 10 | SEQ ID NO: 41 | 25-145 |
| 11 | SEQ ID NO: 43 | 25-143 |
| 12 | SEQ ID NO: 45 | 25-151 |
| 13 | SEQ ID NO: 47 | 25-141 |
| 14 | SEQ ID NO: 49 | 25-140 |
| 15 | SEQ ID NO: 51 | 25-145 |
| 16 | SEQ ID NO: 53 | 25-152 |
| 17 | SEQ ID NO: 55 | 25-142 |
| 18 | SEQ ID NO: 57 | 25-147 |
| 19 | SEQ ID NO: 59 | 25-141 |
| 20 | SEQ ID NO: 61 | 25-148 |
| 21 | SEQ ID NO: 63 | 25-142 |
| 22 | SEQ ID NO: 65 | 25-145 |
| 23 | SEQ ID NO: 67 | 25-140 |
| 24 | SEQ ID NO: 69 | 25-145 |
| 25 | SEQ ID NO: 71 | 25-140 |
| 26 | SEQ ID NO: 73 | 25-140 |
| 27 | SEQ ID NO: 75 | 25-145 |
| 28 | SEQ ID NO: 77 | 25-143 |
| 29 | SEQ ID NO: 79 | 25-143 |
| 30 | SEQ ID NO: 81 | 25-151 |
| 31 | SEQ ID NO: 83 | 25-142 |
| 32 | SEQ ID NO: 85 | 25-144 |
| 33 | SEQ ID NO: 87 | 25-148 |
| 34 | SEQ ID NO: 89 | 25-144 |
| 35 | SEQ ID NO: 91 | 25-140 |
| 36 | SEQ ID NO: 93 | 25-143 |
| 37 | SEQ ID NO: 95 | 25-150 |
| 38 | SEQ ID NO: 97 | 25-147 |
| 39 | SEQ ID NO: 99 | 25-141 |
| 40 | SEQ ID NO: 101 | 25-153 |
| 41 | SEQ ID NO: 103 | 25-152 |
| 42 | SEQ ID NO: 105 | 25-145 |
| 43 | SEQ ID NO: 107 | 25-144 |
| 44 | SEQ ID NO: 109 | 25-146 |
| 45 | SEQ ID NO: 111 | 25-140 |
| 46 | SEQ ID NO: 113 | 25-143 |
| 47 | SEQ ID NO: 115 | 25-144 |
| 48 | SEQ ID NO: 117 | 25-141 |
| 49 | SEQ ID NO: 119 | 25-149 |
| 50 | SEQ ID NO: 121 | 25-145 |
| 51 | SEQ ID NO: 123 | 25-149 |
| 52 | SEQ ID NO: 125 | 25-149 |
| 53 | SEQ ID NO: 127 | 25-147 |
| 54 | SEQ ID NO: 129 | 25-147 |
| 55 | SEQ ID NO: 131 | 25-145 |
| 56 | SEQ ID NO: 133 | 25-146 |
| 57 | SEQ ID NO: 135 | 25-152 |
| 58 | SEQ ID NO: 137 | 25-146 |
| 59 | SEQ ID NO: 139 | 25-149 |
| 60 | SEQ ID NO: 141 | 25-149 |
| 61 | SEQ ID NO: 143 | 25-145 |
| 62 | SEQ ID NO: 145 | 25-142 |
| 63 | SEQ ID NO: 147 | 25-147 |
| 64 | SEQ ID NO: 149 | 25-141 |
| 65 | SEQ ID NO: 151 | 25-140 |
| 66 | SEQ ID NO: 153 | 25-145 |
| 67 | SEQ ID NO: 155 | 25-153 |
| 68 | SEQ ID NO: 157 | 25-146 |
| 69 | SEQ ID NO: 159 | 25-149 |
| 70 | SEQ ID NO: 161 | 25-141 |
| 71 | SEQ ID NO: 163 | 25-156 |
| 72 | SEQ ID NO: 165 | 25-141 |
| 73 | SEQ ID NO: 167 | 25-140 |
| 74 | SEQ ID NO: 169 | 25-140 |
| 75 | SEQ ID NO: 171 | 25-141 |
| 76 | SEQ ID NO: 173 | 25-140 |
| 77 | SEQ ID NO: 175 | 25-144 |
| 78 | SEQ ID NO: 177 | 25-142 |
| 79 | SEQ ID NO: 179 | 25-145 |
| 80 | SEQ ID NO: 181 | 25-145 |
| 81 | SEQ ID NO: 183 | 25-143 |
| 82 | SEQ ID NO: 185 | 25-147 |
| 83 | SEQ ID NO: 187 | 25-143 |
| 84 | SEQ ID NO: 189 | 25-145 |
| 85 | SEQ ID NO: 191 | 25-144 |
| 86 | SEQ ID NO: 193 | 25-143 |
| 87 | SEQ ID NO: 195 | 25-146 |
| 88 | SEQ ID NO: 197 | 25-141 |
| 89 | SEQ ID NO: 199 | 25-146 |
| 90 | SEQ ID NO: 201 | 25-142 |
| 91 | SEQ ID NO: 203 | 25-139 |
| 92 | SEQ ID NO: 205 | 25-144 |
| 93 | SEQ ID NO: 207 | 25-146 |
| 94 | SEQ ID NO: 209 | 25-142 |
| 95 | SEQ ID NO: 211 | 25-151 |
| 96 | SEQ ID NO: 213 | 25-141 |
| 97 | SEQ ID NO: 215 | 25-140 |
| 98 | SEQ ID NO: 217 | 25-146 |
| 99 | SEQ ID NO: 219 | 25-142 |
| 100 | SEQ ID NO: 221 | 25-143 |
| 101 | SEQ ID NO: 223 | 25-150 |
| 102 | SEQ ID NO: 225 | 25-144 |
| 103 | SEQ ID NO: 227 | 25-145 |
| 104 | SEQ ID NO: 229 | 25-149 |
| 105 | SEQ ID NO: 231 | 25-147 |
| 106 | SEQ ID NO: 233 | 25-140 |
| 107 | SEQ ID NO: 235 | 25-141 |
| 108 | SEQ ID NO: 237 | 25-140 |
| 109 | SEQ ID NO: 239 | 25-150 |

TABLE 2C
ST2 antibody IgG1 light chain variable regions. The amino acid
numbers refer to the amino acid positions in the listed sequence.
For example, the IgG1 light chain variable region of Antibody
1 consists of amino acid positions 21-132 of SEQ ID NO: 241.

| Human | IgG1 Light | Amino Acids in |
|---|---|---|
| ST2 IgG1 | Chain Amino | IgG1 Light Chain |
| Antibody | Acid Sequence | Variable Region |
| 1 | SEQ ID NO: 241 | 21-132 |
| 2 | SEQ ID NO: 243 | 21-132 |
| 3 | SEQ ID NO: 245 | 21-128 |
| 4 | SEQ ID NO: 247 | 21-127 |
| 5 | SEQ ID NO: 249 | 21-127 |
| 6 | SEQ ID NO: 251 | 21-127 |
| 7 | SEQ ID NO: 253 | 21-131 |
| 8 | SEQ ID NO: 255 | 21-132 |
| 9 | SEQ ID NO: 257 | 21-127 |
| 10 | SEQ ID NO: 259 | 21-132 |
| 11 | SEQ ID NO: 261 | 21-127 |
| 12 | SEQ ID NO: 263 | 21-127 |
| 13 | SEQ ID NO: 265 | 21-132 |
| 14 | SEQ ID NO: 267 | 21-132 |
| 15 | SEQ ID NO: 269 | 21-127 |
| 16 | SEQ ID NO: 271 | 21-127 |
| 17 | SEQ ID NO: 273 | 21-127 |
| 18 | SEQ ID NO: 275 | 21-127 |
| 19 | SEQ ID NO: 277 | 21-127 |
| 20 | SEQ ID NO: 279 | 21-127 |
| 21 | SEQ ID NO: 281 | 21-127 |
| 22 | SEQ ID NO: 283 | 21-127 |
| 23 | SEQ ID NO: 285 | 21-127 |
| 24 | SEQ ID NO: 287 | 21-132 |
| 25 | SEQ ID NO: 289 | 21-127 |
| 26 | SEQ ID NO: 291 | 21-133 |
| 27 | SEQ ID NO: 293 | 21-127 |
| 28 | SEQ ID NO: 295 | 21-132 |
| 29 | SEQ ID NO: 297 | 21-127 |
| 30 | SEQ ID NO: 299 | 21-132 |
| 31 | SEQ ID NO: 301 | 21-127 |
| 32 | SEQ ID NO: 303 | 21-127 |
| 33 | SEQ ID NO: 305 | 21-132 |
| 34 | SEQ ID NO: 307 | 21-127 |
| 35 | SEQ ID NO: 309 | 21-127 |
| 36 | SEQ ID NO: 311 | 21-127 |
| 37 | SEQ ID NO: 313 | 21-127 |
| 38 | SEQ ID NO: 315 | 21-128 |
| 39 | SEQ ID NO: 317 | 21-132 |
| 40 | SEQ ID NO: 319 | 21-127 |
| 41 | SEQ ID NO: 321 | 21-133 |
| 42 | SEQ ID NO: 323 | 21-127 |
| 43 | SEQ ID NO: 325 | 21-127 |
| 44 | SEQ ID NO: 327 | 21-127 |
| 45 | SEQ ID NO: 329 | 21-127 |
| 46 | SEQ ID NO: 331 | 21-127 |
| 47 | SEQ ID NO: 333 | 21-127 |
| 48 | SEQ ID NO: 335 | 21-127 |
| 49 | SEQ ID NO: 337 | 21-127 |
| 50 | SEQ ID NO: 339 | 21-127 |
| 51 | SEQ ID NO: 341 | 21-128 |
| 52 | SEQ ID NO: 343 | 21-131 |
| 53 | SEQ ID NO: 345 | 21-127 |
| 54 | SEQ ID NO: 347 | 21-131 |
| 55 | SEQ ID NO: 349 | 21-132 |
| 56 | SEQ ID NO: 351 | 21-127 |
| 57 | SEQ ID NO: 353 | 21-127 |
| 58 | SEQ ID NO: 355 | 21-127 |
| 59 | SEQ ID NO: 357 | 21-127 |
| 60 | SEQ ID NO: 359 | 21-127 |
| 61 | SEQ ID NO: 361 | 21-132 |
| 62 | SEQ ID NO: 363 | 21-133 |
| 63 | SEQ ID NO: 365 | 21-132 |
| 64 | SEQ ID NO: 367 | 21-126 |
| 65 | SEQ ID NO: 369 | 21-127 |
| 66 | SEQ ID NO: 371 | 21-132 |
| 67 | SEQ ID NO: 373 | 21-127 |
| 68 | SEQ ID NO: 375 | 21-132 |
| 69 | SEQ ID NO: 377 | 21-132 |
| 70 | SEQ ID NO: 379 | 21-128 |
| 71 | SEQ ID NO: 381 | 21-127 |
| 72 | SEQ ID NO: 383 | 21-127 |
| 73 | SEQ ID NO: 385 | 21-127 |
| 74 | SEQ ID NO: 387 | 21-127 |
| 75 | SEQ ID NO: 389 | 21-127 |
| 76 | SEQ ID NO: 391 | 21-127 |
| 77 | SEQ ID NO: 393 | 21-133 |
| 78 | SEQ ID NO: 395 | 21-127 |
| 79 | SEQ ID NO: 397 | 21-127 |
| 80 | SEQ ID NO: 399 | 21-132 |
| 81 | SEQ ID NO: 401 | 21-127 |
| 82 | SEQ ID NO: 403 | 21-127 |
| 83 | SEQ ID NO: 405 | 21-127 |
| 84 | SEQ ID NO: 407 | 21-132 |
| 85 | SEQ ID NO: 409 | 21-127 |
| 86 | SEQ ID NO: 411 | 21-127 |
| 87 | SEQ ID NO: 413 | 21-127 |
| 88 | SEQ ID NO: 415 | 21-127 |
| 89 | SEQ ID NO: 417 | 21-127 |
| 90 | SEQ ID NO: 419 | 21-132 |
| 91 | SEQ ID NO: 421 | 21-127 |
| 92 | SEQ ID NO: 423 | 21-133 |
| 93 | SEQ ID NO: 425 | 21-132 |
| 94 | SEQ ID NO: 427 | 21-128 |
| 95 | SEQ ID NO: 429 | 21-132 |
| 96 | SEQ ID NO: 431 | 21-132 |
| 97 | SEQ ID NO: 433 | 21-127 |
| 98 | SEQ ID NO: 435 | 21-127 |
| 99 | SEQ ID NO: 437 | 21-127 |
| 100 | SEQ ID NO: 439 | 21-127 |
| 101 | SEQ ID NO: 441 | 21-126 |
| 102 | SEQ ID NO: 443 | 21-132 |
| 103 | SEQ ID NO: 445 | 21-127 |
| 104 | SEQ ID NO: 447 | 21-127 |
| 105 | SEQ ID NO: 449 | 21-127 |
| 106 | SEQ ID NO: 451 | 21-128 |
| 107 | SEQ ID NO: 453 | 21-127 |
| 108 | SEQ ID NO: 455 | 21-127 |
| 109 | SEQ ID NO: 457 | 21-133 |

In certain aspects, the disclosure relates to a recombinant antibody or an antigen-binding fragment thereof that specifically binds ST2, wherein the antibody, or antigen-binding fragment thereof, comprises six complementarity determining regions (CDRs): CDRH1, CDRH2, CDRH3, CDRL1, CDRL2, and CDRL3, wherein CDRH1 comprises an amino acid sequence that has at least 85%, 90%, 95%, 96%, 97%, 98%, or 99% sequence identity to a CDRH1 amino acid sequence as set forth in a heavy chain variable domain amino acid sequence selected from the group consisting of:

amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239,
wherein CDRH2 comprises an amino acid sequence that has at least 85%, 90%, 95%, 96%, 97%, 98%, or 99% sequence identity to a CDRH2 amino acid sequence as set forth in a heavy chain variable domain amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239,
wherein CDRH3 comprises an amino acid sequence that has at least 85%, 90%, 95%, 96%, 97%, 98% or 99% sequence identity to a CDRH3 amino acid sequence as set forth in a heavy chain variable domain amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239,
wherein CDRL1 comprises an amino acid sequence that has at least 85%, 90%, 95%, 96%, 97%, 98%, or 99% sequence identity to a CDRL1 amino acid sequence as set forth in a light chain variable region amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457,
wherein CDRL2 comprises an amino acid sequence that that has at least 85%, 90%, 95%, 96%, 97%, 98%, or 99% sequence identity to a CDRL2 sequence as set forth in a light chain variable region amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457, and
wherein CDRL3 comprises an amino acid sequence that has at least 85%, 90%, 95%, 96%, 97%, 98%, or 99% sequence identity to a CDRL3 sequence as set forth in a light chain variable region amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457.

The CDRH1, CDRH2, CDRH3, CDRL1, CDRL2 and CDRL3 amino acid sequence for human ST2 IgG1 Antibodies 1-109 are provided below in Table 2D.

TABLE 2D
Sequence identifiers for human ST2 IgG1 antibody CDR amino acid
sequences. The CDR sequences are defined according to IMGT.

| Human | CDRH1 | CDRH2 | CDRH3 | CDRL1 | CDRL2 | CDRL3 |
|---|---|---|---|---|---|---|
| ST2 IgG1 | SEQ ID | SEQ ID | SEQ ID | SEQ ID | SEQ ID | SEQ ID |
| Antibody | NO: | NO: | NO: | NO: | NO: | NO: |
| 1 | 458 | 459 | 460 | 461 | 462 | 463 |
| 2 | 464 | 465 | 466 | 467 | 468 | 469 |
| 3 | 470 | 471 | 472 | 473 | 474 | 475 |
| 4 | 476 | 477 | 478 | 479 | 480 | 481 |
| 5 | 482 | 483 | 484 | 485 | 486 | 487 |
| 6 | 488 | 489 | 490 | 491 | 492 | 493 |
| 7 | 494 | 495 | 496 | 497 | 498 | 499 |
| 8 | 500 | 501 | 502 | 503 | 504 | 505 |
| 9 | 506 | 507 | 508 | 509 | 510 | 511 |
| 10 | 512 | 513 | 514 | 515 | 516 | 517 |
| 11 | 518 | 519 | 520 | 521 | 522 | 523 |
| 12 | 524 | 525 | 526 | 527 | 528 | 529 |
| 13 | 530 | 531 | 532 | 533 | 534 | 535 |
| 14 | 536 | 537 | 538 | 539 | 540 | 541 |
| 15 | 542 | 543 | 544 | 545 | 546 | 547 |
| 16 | 548 | 549 | 550 | 551 | 552 | 553 |
| 17 | 554 | 555 | 556 | 557 | 558 | 559 |
| 18 | 560 | 561 | 562 | 563 | 564 | 565 |
| 19 | 566 | 567 | 568 | 569 | 570 | 571 |
| 20 | 572 | 573 | 574 | 575 | 576 | 577 |
| 21 | 578 | 579 | 580 | 581 | 582 | 583 |
| 22 | 584 | 585 | 586 | 587 | 588 | 589 |
| 23 | 590 | 591 | 592 | 593 | 594 | 595 |
| 24 | 596 | 597 | 598 | 599 | 600 | 601 |
| 25 | 602 | 603 | 604 | 605 | 606 | 607 |
| 26 | 608 | 609 | 610 | 611 | 612 | 613 |
| 27 | 614 | 615 | 616 | 617 | 618 | 619 |
| 28 | 620 | 621 | 622 | 623 | 624 | 625 |
| 29 | 626 | 627 | 628 | 629 | 630 | 631 |
| 30 | 632 | 633 | 634 | 635 | 636 | 637 |
| 31 | 638 | 639 | 640 | 641 | 642 | 643 |
| 32 | 644 | 645 | 646 | 647 | 648 | 649 |
| 33 | 650 | 651 | 652 | 653 | 654 | 655 |
| 34 | 656 | 657 | 658 | 659 | 660 | 661 |
| 35 | 662 | 663 | 664 | 665 | 666 | 667 |
| 36 | 668 | 669 | 670 | 671 | 672 | 673 |
| 37 | 674 | 675 | 676 | 677 | 678 | 679 |
| 38 | 680 | 681 | 682 | 683 | 684 | 685 |
| 39 | 686 | 687 | 688 | 689 | 690 | 691 |
| 40 | 692 | 693 | 694 | 695 | 696 | 697 |
| 41 | 698 | 699 | 700 | 701 | 702 | 703 |
| 42 | 704 | 705 | 706 | 707 | 708 | 709 |
| 43 | 710 | 711 | 712 | 713 | 714 | 715 |
| 44 | 716 | 717 | 718 | 719 | 720 | 721 |
| 45 | 722 | 723 | 724 | 725 | 726 | 727 |
| 46 | 728 | 729 | 730 | 731 | 732 | 733 |
| 47 | 734 | 735 | 736 | 737 | 738 | 739 |
| 48 | 740 | 741 | 742 | 743 | 744 | 745 |
| 49 | 746 | 747 | 748 | 749 | 750 | 751 |
| 50 | 752 | 753 | 754 | 755 | 756 | 757 |
| 51 | 758 | 759 | 760 | 761 | 762 | 763 |
| 52 | 764 | 765 | 766 | 767 | 768 | 769 |
| 53 | 770 | 771 | 772 | 773 | 774 | 775 |
| 54 | 776 | 777 | 778 | 779 | 780 | 781 |
| 55 | 782 | 783 | 784 | 785 | 786 | 787 |
| 56 | 788 | 789 | 790 | 791 | 792 | 793 |
| 57 | 794 | 795 | 796 | 797 | 798 | 799 |
| 58 | 800 | 801 | 802 | 803 | 804 | 805 |
| 59 | 806 | 807 | 808 | 809 | 810 | 811 |
| 60 | 812 | 813 | 814 | 815 | 816 | 817 |
| 61 | 818 | 819 | 820 | 821 | 822 | 823 |
| 62 | 824 | 825 | 826 | 827 | 828 | 829 |
| 63 | 830 | 831 | 832 | 833 | 834 | 835 |
| 64 | 836 | 837 | 838 | 839 | 840 | 841 |
| 65 | 842 | 843 | 844 | 845 | 846 | 847 |
| 66 | 848 | 849 | 850 | 851 | 852 | 853 |
| 67 | 854 | 855 | 856 | 857 | 858 | 859 |
| 68 | 860 | 861 | 862 | 863 | 864 | 865 |
| 69 | 866 | 867 | 868 | 869 | 870 | 871 |
| 70 | 872 | 873 | 874 | 875 | 876 | 877 |
| 71 | 878 | 879 | 880 | 881 | 882 | 883 |
| 72 | 884 | 885 | 886 | 887 | 888 | 889 |
| 73 | 890 | 891 | 892 | 893 | 894 | 895 |
| 74 | 896 | 897 | 898 | 899 | 900 | 901 |
| 75 | 902 | 903 | 904 | 905 | 906 | 907 |
| 76 | 908 | 909 | 910 | 911 | 912 | 913 |
| 77 | 914 | 915 | 916 | 917 | 918 | 919 |
| 78 | 920 | 921 | 922 | 923 | 924 | 925 |
| 79 | 926 | 927 | 928 | 929 | 930 | 931 |
| 80 | 932 | 933 | 934 | 935 | 936 | 937 |
| 81 | 938 | 939 | 940 | 941 | 942 | 943 |
| 82 | 944 | 945 | 946 | 947 | 948 | 949 |
| 83 | 950 | 951 | 952 | 953 | 954 | 955 |
| 84 | 956 | 957 | 958 | 959 | 960 | 961 |
| 85 | 962 | 963 | 964 | 965 | 966 | 967 |
| 86 | 968 | 969 | 970 | 971 | 972 | 973 |
| 87 | 974 | 975 | 976 | 977 | 978 | 979 |
| 88 | 980 | 981 | 982 | 983 | 984 | 985 |
| 89 | 986 | 987 | 988 | 989 | 990 | 991 |
| 90 | 992 | 993 | 994 | 995 | 996 | 997 |
| 91 | 998 | 999 | 1000 | 1001 | 1002 | 1003 |
| 92 | 1004 | 1005 | 1006 | 1007 | 1008 | 1009 |
| 93 | 1010 | 1011 | 1012 | 1013 | 1014 | 1015 |
| 94 | 1016 | 1017 | 1018 | 1019 | 1020 | 1021 |
| 95 | 1022 | 1023 | 1024 | 1025 | 1026 | 1027 |
| 96 | 1028 | 1029 | 1030 | 1031 | 1032 | 1033 |
| 97 | 1034 | 1035 | 1036 | 1037 | 1038 | 1039 |
| 98 | 1040 | 1041 | 1042 | 1043 | 1044 | 1045 |
| 99 | 1046 | 1047 | 1048 | 1049 | 1050 | 1051 |
| 100 | 1052 | 1053 | 1054 | 1055 | 1056 | 1057 |
| 101 | 1058 | 1059 | 1060 | 1061 | 1062 | 1063 |
| 102 | 1064 | 1065 | 1066 | 1067 | 1068 | 1069 |
| 103 | 1070 | 1071 | 1072 | 1073 | 1074 | 1075 |
| 104 | 1076 | 1077 | 1078 | 1079 | 1080 | 1081 |
| 105 | 1082 | 1083 | 1084 | 1085 | 1086 | 1087 |
| 106 | 1088 | 1089 | 1090 | 1091 | 1092 | 1093 |
| 107 | 1094 | 1095 | 1096 | 1097 | 1098 | 1099 |
| 108 | 1100 | 1101 | 1102 | 1103 | 1104 | 1105 |
| 109 | 1106 | 1107 | 1108 | 1109 | 1110 | 1111 |

In some embodiments, the disclosure relates to an isolated antibody or antigen-binding fragment thereof that specifically binds ST2, comprising a heavy chain variable domain comprising complementarity determining regions (CDRs) as set forth in a heavy chain variable domain amino acid sequence selected from the group consisting of:

amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239; and

comprising a light chain variable domain comprising CDRs as set forth in a light chain variable region amino acid sequence selected from the group consisting of:

amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457.

In some aspects, the disclosure relates to a recombinant antibody or an antigen-binding fragment thereof that specifically binds ST2, wherein the antibody, or antigen-binding fragment thereof, comprises six complementarity determining regions (CDRs): CDRH1, CDRH2, CDRH3, CDRL1, CDRL2, and CDRL3 as set forth in Table 2D. For example, in some embodiments, the antibody or antigen-binding fragment thereof comprises the six complementarity determining regions (CDRs): CDRH1, CDRH2, CDRH3, CDRL1, CDRL2, and CDRL3 of Antibody1, the six complementarity determining regions (CDRs): CDRH1, CDRH2, CDRH3, CDRL1, CDRL2, and CDRL3 of Antibody 2, or the six complementarity determining regions (CDRs): CDRH1, CDRH2, CDRH3, CDRL1, CDRL2, and CDRL3 of Antibody 3, etc., as set forth in Table 2D. In some embodiments, the CDRH1 comprises an amino acid sequence that has at least 85%, 90%, 95%, 96%, 97%, 98%, or 99% sequence identity to a CDRH1 amino acid sequence set forth in Table 2D. In some embodiments, the CDRH2 comprises an amino acid sequence that has at least 85%, 90%, 95%, 96%, 97%, 98%, or 99% sequence identity to a CDRH2 amino acid sequence set forth in Table 2D. In some embodiments, the CDRH3 comprises an amino acid sequence that has at least 85%, 90%, 95%, 96%, 97%, 98%, or 99% sequence identity to a CDRH3 amino acid sequence set forth in Table 2D. In some embodiments, the CDRL1 comprises an amino acid sequence that has at least 85%, 90%, 95%, 96%, 97%, 98%, or 99% sequence identity to a CDRL1 amino acid sequence set forth in Table 2D. In some embodiments, the CDRL2 comprises an amino acid sequence that has at least 85%, 90%, 95%, 96%, 97%, 98%, or 99% sequence identity to a CDRL2 amino acid sequence set forth in Table 2D. In some embodiments, the CDRL3 comprises an amino acid sequence that has at least 85%, 90%, 95%, 96%, 97%, 98%, or 99% sequence identity to a CDRL3 amino acid sequence set forth in Table 2D.

### Linkage Between an IL-2R-Binding Moiety and an ST2-Binding Moiety

The IL-2R and ST2 binding moieties are linked. A first moiety that binds to the IL-2 receptor and a second moiety that binds to ST2 are linked covalently or non-covalently. In some embodiments, the first moiety that binds to the IL-2 receptor and the second moiety that binds to ST2 are covalently linked. For example, the first moiety that binds to the IL-2 receptor and the second moiety that binds to ST2 can be covalently linked by a sulfide bond or a disulfide bond. In some embodiments, a compound comprising a first moiety that binds to the IL-2 receptor and a second moiety that binds to ST2 comprises a multimerization moiety or two multimerization moieties, for example, Fc domains. For example, a first multimerization moiety can be covalently linked to the first moiety that binds to the IL-2 receptor and a second multimerization moiety can be covalently linked to the second moiety that binds to ST2. The two multimerization moieties also can be covalently linked to each other. In some embodiments, the two multimerization moieties are polypeptide sequences. For example, in some embodiments, a disulfide bond covalently links a first Fc domain that is covalently linked to the IL-2R-binding moiety and a second Fc domain that is covalently linked to the ST2-binding moiety.

### Immunoglobulin Fc Domains

In some embodiments, a multimerization moiety is an immunoglobulin Fc domain, for example, an immunoglobulin Fc domain that is deficient in effector functions relative to a corresponding wild-type immunoglobulin Fc domain. Non-limiting examples of immunoglobulin Fc domains are IgG, IgA, IgD, IgM, and IgE immunoglobulin Fc domains. In some embodiments, an immunoglobulin Fc domains is an IgG1 immunoglobulin Fc domain.

Immunoglobulin Fc domains have a number of therapeutic benefits when incorporated into fusion proteins. For example, immunoglobulin Fc domains can increase the circulating half-life of the fusion partner protein.

In some embodiments, the increased circulating half-life is due to the Fc domain preventing aggregation of the fusion protein, thereby increasing its stability and slowing clearance.

The four human IgG subclasses differ in effector functions (CDC, ADCC), circulating half-life, and stability. IgG1 possesses Fc effector functions, and is the most abundant IgG subclass. IgG2 is deficient in Fc effector functions, but is subject to both dimerization with other IgG2 molecules, and instability due to scrambling of disulfide bonds in the hinge region. IgG3 possesses Fc effector functions, and has a long, rigid hinge region. IgG4 is deficient in Fc effector functions, and has a shorter circulating half-life than the other subclasses. The IgG4 dimer is biochemically unstable due to having only a single disulfide bond in the hinge region leading to the exchange of H chains between different IgG4 molecules. Fc sequence modifications can be made to the hinge region of an IgG2 Fc to prevent aggregation, or to the hinge region of an IgG4 Fc to stabilize dimers.

Effector function-deficient variants of IgG1 can be generated. For example, an amino acid substitution can be made at position N297, the location of an N-linked glycosylation site. In some embodiments, the substitution is N297A. Substitution of this asparagine residue removes the glycosylation site and significantly reduces antibody-dependent cell-mediated cytotoxicity (ADCC) and complement-dependent cytotoxicity (CDC) activity, thereby preventing unwanted cell lysis.

Various other effector function-deficient IgG1 variants can also be appreciated by the skilled worker. One non-limiting example of such a variant is IgG1 (L234F/L235E/P331S), which mutates amino acids in the Clq and FcyR binding sites. These (or similar) Fc variants can be used to generate effector-deficient and stable IL-2 selective agonist-Fc fusion proteins (IL2SA-Fc). Forms of Fc protein moieties also can be engineered to create stable monomers rather than dimers. These modified Fc protein moieties also can be combined with an IL-2 compound of the present disclosure. Additionally, a functionally monomeric heterodimer comprising an IL-2-Fc H chain polypeptide can be combined with an Fc H chain polypeptide and assembled using bispecific antibody technology with an IL-2 selective agonist. IL-2 Fc fusion proteins also can be made with intact IgG antibody molecules, either with or without antigen specificity in the IgG moiety. Moreover, Fc variants that lack some of the hinge region can be used with the compounds and methods described herein.

In some embodiments, the sequence of an immunoglobulin Fc moiety is an IgG1 Fc moiety comprising an N297A mutation, for example, the sequence shown below:

(SEQ ID NO: 7; N297A mutation
is shown in bold and underlined)
DKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFS
CSVMHEALHNHYTQKSLSLSPG.

In some embodiments, the IgG1 Fc moiety has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 7.

The compound of the present disclosure can be produced under conditions that allow two Ig polypeptides to form an Fc domain. The two Ig polypeptides can be conjugated with different moieties. In some cases one IgG polypeptide is conjugated to an IL-2 moiety and the second Ig polypeptide is bound to a moiety that binds a cell surface protein other than the IL2 receptor. In some embodiments the cell surface protein bound by the binding moiety is ST2.

### Linker

The linkage at the junction between an Fc domain and an IL2 receptor-binding moiety or an ST2-binding moiety can be: (1) a direct fusion of the two protein sequences; (2) a fusion with an intervening linker peptide; or (3) a fusion by a non-peptide moiety. In some embodiments, a linker directly links an IL2R-binding moiety and an ST2-binding moiety. Linker peptides can be included as spacers between two protein moieties. Linker peptides can promote proper protein folding, stability, expression, and bioactivity of the component protein moieties. Long flexible linker peptides can be composed of glycine, serine, or threonine, with multiple glycine residues providing a highly flexible conformation. Serine or threonine residues provide polar surface area to limit hydrophobic interaction within the peptide or with the component fusion protein moieties. In some embodiments, peptide linkers are rich in glycine and serine, such as repeats of the sequence GGGGS (SEQ ID NO: 21). In some embodiments, a peptide linker has a sequence of (GGGGS)ₙ (SEQ ID NO: 21), wherein n is 1, 2, 3, 4, 5, 6, 7, 8, 9, or 10. In some embodiments, n is 3; i.e., a peptide linker has a sequence of GGGGSGGGGSGGGGS (SEQ ID NO: 6). In some embodiments, the IL-2 receptor-binding moiety is N-terminal to the linker peptide, and the immunoglobulin Fc domain is C-terminal to the linker peptide. In some embodiments, the IL-2 receptor-binding moiety is C-terminal to the linker peptide, and the immunoglobulin Fc domain is N-terminal to the linker peptide.

In some embodiments, the peptide linker has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of SEQ ID NO: 6.

### Pharmaceutical Compositions

A pharmaceutical composition of the invention can comprise any compound described herein. In some embodiments, a pharmaceutical composition comprises a compound of the present disclosure with other chemical components, such as carriers, stabilizers, diluents, dispersing agents, suspending agents, thickening agents, and/or excipients. The pharmaceutical composition facilitates administration of a compound of the present disclosure to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining a compound of the present disclosure with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a compound of the present disclosure in water-soluble form. Suspensions of a compound of the present disclosure can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

A compound of the present disclosure can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

A compound of the present disclosure can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of a compound of the present disclosure into preparations that can be used pharmaceutically. Formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after an antibiotic. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Diseases

The compounds of the present disclosure can be applied to various autoimmune or immune-related diseases or conditions, for example to treat such diseases or conditions. For example, the present disclosure provides a method for treating a condition, the method comprising administering to a subject in need thereof a therapeutically-effective amount of a compound of the present disclosure. In some embodiments, the compound administered to the subject in need thereof comprises a first moiety that binds IL-2R and a second moiety that binds ST2, wherein the first moiety is covalently linked via a linker to a first Fc domain and the second moiety is covalently linked via a linker to a second Fc domain, and the first and second Fc domains are covalently linked, and further wherein the first and second Fc domains are deficient in effector functions.

Autoimmune diseases include diseases that affect organs such as the heart, kidney, liver, lung, reproductive organs, digestive system, or skin. Autoimmune diseases include diseases that affect glands, including the endocrine, adrenal, thyroid, salivary and exocrine glands, and the pancreas. Autoimmune diseases can also be multi-glandular. Autoimmune diseases can target one or more tissues, for example connective tissue, muscle, or blood. Autoimmune diseases can target the nervous system or eyes, ears or vascular system. Autoimmune diseases can also be systemic, affecting multiple organs, tissues and/or systems. In some embodiments, an immune-related disease or condition is an inflammatory disease or condition. In some embodiments, an inflammatory disease or condition is one which involves inflamed muscle, visceral adipose, colon, and/or lung tissue.

In certain embodiments, the autoimmune disease is selected from the group consisting of Graft-vs-Host Disease, Pemphigus Vulgaris, Systemic Lupus Erythematosus, Scleroderma, Ulcerative Colitis, Crohn's Disease, Psoriasis, Type 1 Diabetes, Multiple Sclerosis, Amyotrophic Lateral Sclerosis, Alopecia Areata, Uveitis, Neuromyelitis Optica, and Duchenne Muscular Dystrophy.

In some embodiments, the compounds of the present disclosure treat diseases affecting muscle tissue, for example, inflammatory myopathies, muscular dystrophies, muscle diseases with immune system involvement, and muscle diseases involving inflammation.

Inflammatory myopathies are diseases that typically involve inflammation of the muscles and associated symptoms, such as muscle weakness. The muscle weakness can be progressive. Symptoms associated with inflammatory myopathies (e.g., dermatomyositis) can include, for example, muscle weakness (e.g. proximal muscle weakness), skin rash, fatigue after walking or standing, tripping or falling, dysphagia, dysphonia, difficulty breathing, muscle pain, tender muscles, weight loss, low-grade fever, inflamed lungs, light sensitivity, calcium deposits (calcinosis) under the skin or in the muscle, and biological concomitants of inflammatory myopathies.

Inflammatory myopathies can be caused by allergic reactions, other diseases, exposure to a drug or toxin, or exposure to an infectious agent, or can be idiopathic (no known cause). The inflammatory myopathy can be an acute inflammatory myopathy or a chronic inflammatory myopathy. Inflammatory myopathies can affect both adults and children (e.g., juvenile dermatomyositis). Inflammatory myopathies can include symptoms that affect other organs or systems of the body, such as the skin, lungs, heart, eyes, and gastrointestinal system. In some embodiments, the inflammatory myopathy is a chronic inflammatory myopathy (e.g., dermatomyositis, polymyositis, or inclusion body myositis).

In some embodiments, the inflammatory myopathy can be caused by an allergic reaction, another disease (e.g., cancer or a connective tissue disease), exposure to a toxic substance, a medicine, or an infectious agent (e.g., a virus). In some embodiments, the inflammatory myopathy is associated with lupus, rheumatoid arthritis, or systemic sclerosis. In some embodiments, the inflammatory myopathy is idiopathic. In some embodiments, the inflammatory myopathy is selected from polymyositis, dermatomyositis, inclusion body myositis, and immune-mediated necrotizing myopathy. In some embodiments, the inflammatory myopathy is dermatomyositis.

Biological concomitants of inflammatory myopathies (e.g., dermatomyositis) include, for example, altered (for example, increased) levels of cytokines (for example, Type I interferons (such as IFN-α and/or IFN-β), interleukins (such as IL-6, IL-10, IL-15, IL-17 and IL-18), and TNF-α), TGF-β, B-cell activating factor (BAFF), and overexpression of IFN inducible genes (for example, Type I IFN inducible genes). Other biological concomitants of inflammatory myopathies can include, for example, an increased erythrocyte sedimentation rate (ESR) and/or elevated level of creatine kinase. Further biological concomitants of inflammatory myopathies can include autoantibodies, for example, anti-synthetase autoantibodies (for example, anti-Jo1 antibodies), anti-signal recognition particle antibodies (anti-SRP), anti-Mi-2 antibodies, anti-p155 antibodies, anti-PM/Sci antibodies, and anti-RNP antibodies.

The muscular dystrophies are a group of diverse, heritable neuromuscular disorders that represent a group of devastating neuromuscular diseases characterized by primary or secondary skeletal muscle involvement. Examples of muscular dystrophies include, but are not limited to, Duchenne muscular dystrophy, Beckers muscular dystrophy, Limb-Girdle muscular dystrophy, Facioscapulohumeral muscular dystrophy, Fukuyama congenital muscular dystrophy, and merosin-deficient congenital muscular dystrophy. The most common form of muscular dystrophy is Duchenne Muscular Dystrophy, (DMD). DMD is an X-linked recessive disorder characterized by a mutation in the gene that codes for dystrophin. Most patients die before age 30 due to respiratory or cardiac failure. Beckers muscular dystrophy (also known as benign pseudohypertrophic muscular dystrophy) is related to DMD in that both result from a mutation in the dystrophin gene. An organism suffering from DMD does not produce functional dystrophin. Thus, DMD is much more severe than BMD.

### Subjects

The compounds of the present disclosure are administered to a subject in need thereof, such as a vertebrate. In some embodiments the subject is a mouse, rat, rabbit, dog, cat, horse, sheep, cow, monkey, cynomolgus monkey, or human. Subjects can be, for example, elderly adults, adults, adolescents, pre-adolescents, children, toddlers, and infants. In some embodiments, the subject is an animal model of an inflammatory myopathy. In some embodiments, the subject is a human with an inflammatory myopathy, or a human at risk of developing an inflammatory myopathy. In some embodiments, the subject has a family history of inflammatory myopathy. In some embodiments the subject carries a gene associated with an inflammatory myopathy. In some embodiments the subject is positive for a biomarker associated with an inflammatory myopathy. In some embodiments, the subject has been diagnosed with an inflammatory myopathy. In some embodiments, the subject has one or more signs or symptoms associated with an inflammatory myopathy, e.g., one or more of the symptoms described herein.

In some embodiments the subject is an animal model of a muscular dystrophy. In some embodiments the subject is a human with a muscular dystrophy, or a human at risk of developing a muscular dystrophy. In some embodiments, the subject has a family history of muscular dystrophy. In some embodiments the subject carries a gene associated with a muscular dystrophy. In some embodiments the subject is positive for a biomarker associated with a muscular dystrophy. In some embodiments, the subject has been diagnosed with a muscular dystrophy. In some embodiments, the subject has one or more signs or symptoms associated with a muscular dystrophy, e.g., one or more of the symptoms described herein.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi dose containers with a preservative.

A compound described herein can be present in a composition in a range of from about 0.5 μg to about 7000 μg, from about 1 μg to about 1000 μg, from about 1 μg to about 250 μg, from about 1 μg to about 25 μg, from about 5 μg to about 50 μg, from about 0.5 μg to about 15 μg, or from about 0.5 μg to about 10 μg per dose.

A compound described herein can be present in a composition in an amount of about 0.5 μg, about 1 μg, about 2 μg, about 3 μg, about 4 μg, about 5 μg, about 6 μg, about 7 μg, about 8 μg, about 9 μg, about 10 μg, about 11 μg, about 12 μg, about 13 μg, about 14 μg, about 15 μg, about 16 μg, about 17 μg, about 18 μg, about 19 μg, about 20 μg, about 21 μg, about 22 μg, about 23 μg, about 24 μg, about 25 μg, about 26 μg, about 27 μg, about 28 μg, about 29 μg, about 30 μg, about 31 μg, about 32 μg, about 33 μg, about 34 μg, about 35 μg, about 36 μg, about 37 μg, about 38 μg, about 39 μg, about 40 μg, about 41 μg, about 42 μg, about 43 μg, about 44 μg, about 45 μg, about 46 μg, about 47 μg, about 48 μg, about 49 μg, about 50 μg, about 55 μg, about 60 μg, about 65 μg, about 70 μg, about 75 μg, about 80 μg, about 85 μg, about 90 μg, about 95 μg, about 100 μg, about 125 μg, about 150 μg, about 175 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1350 μg, about 1400 μg, about 1450 μg, about 1500 μg, about 1550 μg, about 1600 μg, about 1650 μg, about 1700 μg, about 1750 μg, about 1800 μg, about 1850 μg, about 1900 μg, about 1950 μg, about 2000 μg, about 2500 μg, about 3000 μg, about 3500 μg, about 4000 μg, about 4500 μg, about 5000 μg, about 5500 μg, about 6000 μg, about 6500 μg, about 7000 μg, about 7500 μg, about 8000 μg, about 9000 μg, about 10,000 μg (10 mg), about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, or about 40 mg. Any of these values may be used to define a range for the amount of the compound in the composition. For example, the compound may be present in a composition in the range of from about 0.5 μg to about 40 mg, from about 500 μg to about 10 mg, or from about 50 μg to about 5 mg.

In some embodiments, a dose can be expressed in terms of an amount of the drug divided by the mass of the subject, for example, micrograms or milligrams of drug per kilograms of subject body mass. In some embodiments, the compound is administered at a dose of about 0.5 μg/kg, about 1 μg/kg, about 2 μg/kg, about 3 μg/kg, about 4 μg/kg, about 5 μg/kg, about 6 μg/kg, about 7 μg/kg, about 8 μg/kg, about 9 μg/kg, about 10 μg/kg, about 11 μg/kg, about 12 μg/kg, about 13 μg, about 14 μg/kg, about 15 μg/kg, about 16 μg/kg, about 17 μg/kg, about 18 μg/kg, about 19 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, about 125 μg/kg, about 150 μg/kg, about 175 μg/kg, about 200 μg/kg, about 250 μg/kg, about 300 μg/kg, about 350 μg/kg, about 400 μg/kg, about 450 μg/kg, about 500 μg/kg, about 550 μg/kg, about 600 μg/kg, about 650 μg/kg, about 700 μg/kg, about 750 μg/kg, about 800 μg/kg, about 850 μg/kg, about 900 μg/kg, about 950 μg/kg, about 1000 μg/kg, about 1050 μg/kg, about 1100 μg/kg, about 1150 μg/kg, about 1200 μg/kg, about 1250 μg/kg, about 1300 μg/kg, about 1350 μg/kg, about 1400 μg/kg, about 1450 μg/kg, about 1500 μg/kg, about 1550 μg/kg, about 1600 μg/kg, about 1650 μg/kg, about 1700 μg/kg, about 1750 μg/kg, about 1800 μg/kg, about 1850 μg/kg, about 1900 μg/kg, about 1950 μg/kg, about 2000 μg/kg, about 2500 μg/kg, about 3000 μg/kg, about 3500 μg/kg, about 4000 μg/kg, about 4500 μg/kg, or about 5000 μg/kg. Any of these values may be used to define a range for the dose of the compound. For example, in some embodiments, a compound is administered at a dose ranging from about 0.5 μg/kg to about 250 μg/kg, 1 μg/kg to about 200 μg/kg, 5 μg/kg to about 150 μg/kg, about 10 μg/kg to about 100 μg/kg, about 10 μg/kg to about 50 μg/kg, about 15 μg/kg to about 35 μg/kg, or about 0.5 μg/kg to about 5000 μg/kg.

The disclosed compounds can be administered at any interval desired. For example, the compound can be administered once a week, 2 times a week, 3 times a week, 4 times a week, 5 times a week, 6 times a week, 7 times a week, 8 times a week, 9 times a week, or 10 times a week. The interval between daily dosing can be any hourly interval, for example, every hour, every 2 hours, every 3 hours, every 4 hours, every 5 hours, every 6 hours, every 7 hours, every 8 hours, every 9 hours, every 10 hours, every 11 hours, or every 12 hours. The compound can be administered once every week, once every 2 weeks, once every 3 weeks, once every 4 weeks, once every 5 weeks, once every 6 weeks, once every 7 weeks, or once every 8 weeks. The administration of the compound can have irregular dosing schedules to accommodate either the person administering the compound or the subject receiving the compound. As such, the compound can be administered, for example, once a day, twice a day, or three times a day.

The amount administered can be the same amount in each dose or the dosage can vary. For example, a first amount can be dosed in the morning and a second amount can be administered in the evening. A subject could receive a high first dose and lower subsequent doses. The dose can be adjusted up or down depending on improvement in symptoms or markers of the disease, or development of adverse reactions.

Non-limiting examples of pharmaceutically-acceptable carriers include saline, Ringer's solution and dextrose solution. Liquid carriers can be used in preparing solutions, suspensions, and emulsions. A compound described herein can be dissolved or suspended in a pharmaceutically-acceptable liquid carrier such as water, an organic solvent, or a mixture of both, or pharmaceutically-acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for parenteral administration include water, alcohols (including monohydric alcohols and polyhydric alcohols, e.g., glycols) and derivatives thereof, and oils (e.g., fractionated coconut oil and arachis oil). For parenteral administration, the carrier can be an oily ester such as ethyl oleate or isopropyl myristate. Sterile liquid carriers are used in sterile liquid form compositions for parenteral administration. The pH of the solution is can be from about 5 to about 8, for example, from about 7 to about 7.5.

In some embodiments, treatment with a molecule of the present disclosure is better tolerated than is treatment with a wildtype IL-2 polypeptide. In some embodiments, treatment with a therapeutically-effective dose of a molecule of the present disclosure causes fewer incidents of diarrhea relative to treatment with IL2(C125S). In some embodiments, treatment with a therapeutically-effective amount of a molecule of the present disclosure does not cause capillary leak syndrome. In some embodiments, treatment with a therapeutically-effective amount of a molecule of the present disclosure does not cause decreased neutrophil activity or increased risk of infection.

The compounds of the present disclosure have high, moderate, or low affinity for the IL-2 receptor. The compounds of the present disclosure have high, moderate, or low affinity for ST2. A compound that has moderate or low affinity for IL2R and ST2 individually can have high avidity when both receptors are present on a cell. A compound of the present disclosure has a dissociation constant (Kd) of, for example, from about 1 pmol to about 1 mmol, from about 10 pmol to about 1 mmol, from about 100 pmol to about 1 mmol, from about 1 μmol to about 1 mmol, from about 10 μmol to about 1 mmol, from about 1 μmol to about 100 μmol, from about 1 μmol to about 500 μmol, from about 200 μmol to about 800 μmol, from about 10 μmol to about 100 μmole, or from about 500 μmole to about 1 mmol for binding to either ST2 or IL2R individually. A compound of the present disclosure can have a lower apparent Kd when binding to both ST2 and IL-2R, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 4%, less than 3%, less than 2%, or less than 1% of an individual Kd.

### Pharmacokinetics

A dose can be modulated to achieve a desired pharmacokinetic (PK) or pharmacodynamics profile, such as a desired or effective blood profile, as described herein.

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined, for example, by calculating the average of all subject's measurements for each parameter measured. A dose can be modulated to achieve a desired pharmacokinetic or pharmacodynamics profile, such as a desired or effective blood profile, as described herein.

The pharmacodynamic parameters can be any parameters suitable for describing compositions of the invention. For example, the pharmacodynamic profile can be obtained at a time after dosing of, for example, about zero minutes, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 36 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about zero hours, about 0.5 hours, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 2 days, about 3 days, about 4 days, about 5 days, about 6 days, about 7 days, about 8 days, about 9 days, about 10 days, about 11 days, about 12 days, about 13 days, or about 14 days.

The pharmacokinetic parameters can be any parameters suitable for describing a compound. The Cₘₐₓ can be, for example, not less than about 1 ng/mL; not less than about 5 ng/mL; not less than about 10 ng/mL; not less than about 15 ng/mL; not less than about 20 ng/mL; not less than about 25 ng/mL; not less than about 50 ng/mL; not less than about 75 ng/mL; not less than about 100 ng/mL; not less than about 200 ng/mL; not less than about 300 ng/mL; not less than about 400 ng/mL; not less than about 500 ng/mL; not less than about 600 ng/mL; not less than about 700 ng/mL; not less than about 800 ng/mL; not less than about 900 ng/mL; not less than about 1000 ng/mL; not less than about 1250 ng/mL; not less than about 1500 ng/mL; not less than about 1750 ng/mL; not less than about 2000 ng/mL; not less than about 2500 ng/mL; or any other Cₘₐₓ appropriate for describing a pharmacokinetic profile of a compound described herein. The Cₘₐₓ can be, for example, about 5 to about 10,000 ng/mL, about 50 to about 10,000 ng/mL, about 500 to about 10,000 ng/mL, about 5000 to about 10,000 ng/mL, about 1000 to about 5,000 ng/mL, about 1000 to about 3,000 ng/mL, about 5,000 to about 8,000 ng/mL or about 500 to about 1000 ng/mL in blood when administered by intravenous injection, for example, at 50 μg/kg. The Cₘₐₓ can be, for example, about 5 to about 50 ng/mL, about 50 to about 500 ng/mL, about 100 to about 250 ng/mL, about 1000 to about 5000 ng/mL, about 1000 to about 2000 ng/mL, about 2000 to about 5000 ng/mL, about 5000 to about 10000 ng/mL or about 5000 to about 7000 ng/mL in blood when administered by subcutaneous injection, for example, at 50 μg/kg. The Cmax can depend on the dose of compound received. The dose received can be 50 μg/kg, 100 μg/kg, 200 μg/kg, 250 μg/kg, 300 μg/kg, 400 μg/kg, 500 μg/kg, 600 μg/kg, 700 μg/kg, 800 μg/kg, 900 μg/kg, or 1000 μg/kg.

The Tₘₐₓ of a compound described herein can be, for example, not greater than about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, not greater than about 5.5 hours, not greater than about 6 hours, not greater than about 6.5 hours, not greater than about 7 hours, not greater than about 7.5 hours, not greater than about 8 hours, not greater than about 8.5 hours, not greater than about 9 hours, not greater than about 9.5 hours, not greater than about 10 hours, not greater than about 10.5 hours, not greater than about 11 hours, not greater than about 11.5 hours, not greater than about 12 hours, not greater than about 12.5 hours, not greater than about 13 hours, not greater than about 13.5 hours, not greater than about 14 hours, not greater than about 14.5 hours, not greater than about 15 hours, not greater than about 15.5 hours, not greater than about 16 hours, not greater than about 16.5 hours, not greater than about 17 hours, not greater than about 17.5 hours, not greater than about 18 hours, not greater than about 18.5 hours, not greater than about 19 hours, not greater than about 19.5 hours, not greater than about 20 hours, or any other Tₘₐₓ appropriate for describing a pharmacokinetic profile of a compound described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours.

The AUC_{(0-inf)} (also called AUC_{(0-∞)}) or AUC₍ₗₐₛₜ₎ of a compound described herein can be, for example, not less than about 1 ng·hr/mL, not less than about 5 ng·hr/mL, not less than about 10 ng·hr/mL, not less than about 20 ng·hr/mL, not less than about 30 ng·hr/mL, not less than about 40 ng·hr/mL, not less than about 50 ng·hr/mL, not less than about 100 ng·hr/mL, not less than about 150 ng·hr/mL, not less than about 200 ng·hr/mL, not less than about 250 ng·hr/mL, not less than about 300 ng·hr/mL, not less than about 350 ng·hr/mL, not less than about 400 ng·hr/mL, not less than about 450 ng·hr/mL, not less than about 500 ng·hr/mL, not less than about 600 ng·hr/mL, not less than about 700 ng·hr/mL, not less than about 800 ng·hr/mL, not less than about 900 ng·hr/mL, not less than about 1000 ng·hr/mL, not less than about 1250 ng·hr/mL, not less than about 1500 ng·hr/mL, not less than about 1750 ng·hr/mL, not less than about 2000 ng·hr/mL, not less than about 2500 ng·hr/mL, not less than about 3000 ng·hr/mL, not less than about 3500 ng·hr/mL, not less than about 4000 ng·hr/mL, not less than about 5000 ng·hr/mL, not less than about 6000 ng·hr/mL, not less than about 7000 ng·hr/mL, not less than about 8000 ng·hr/mL, not less than about 9000 ng·hr/mL, not less than about 10,000 ng·hr/mL, not less than about 11,000 ng·hr/mL, not less than about 12,000 ng·hr/mL, not less than about 13,000 ng·hr/mL, not less than about 14,000 ng·hr/mL, not less than about 15,000 ng·hr/mL, not less than about 16,000 ng·hr/mL, not less than about 17,000 ng·hr/mL, not less than about 18,000 ng·hr/mL, not less than about 19,000 ng·hr/mL, not less than about 20,000 ng·hr/mL, or any other AUC_{(0-inf)} appropriate for describing a pharmacokinetic profile of a compound described herein. The AUC_{(0-inf)} of a compound can be, for example, about 1 ng·hr/mL to about 10,000 ng·hr/mL; about 1 ng·hr/mL to about 10 ng·hr/mL; about 10 ng·hr/mL to about 25 ng·hr/mL; about 25 ng·hr/mL to about 50 ng·hr/mL; about 50 ng·hr/mL to about 100 ng·hr/mL; about 100 ng·hr/mL to about 200 ng·hr/mL; about 200 ng·hr/mL to about 300 ng·hr/mL; about 300 ng·hr/mL to about 400 ng·hr/mL; about 400 ng·hr/mL to about 500 ng·hr/mL; about 500 ng·hr/mL to about 600 ng·hr/mL; about 600 ng·hr/mL to about 700 ng·hr/mL; about 700 ng·hr/mL to about 800 ng·hr/mL; about 800 ng·hr/mL to about 900 ng·hr/mL; about 900 ng·hr/mL to about 1,000 ng·hr/mL; about 1,000 ng·hr/mL to about 1,250 ng·hr/mL; about 1,250 ng·hr/mL to about 1,500 ng·hr/mL; about 1,500 ng·hr/mL to about 1,750 ng·hr/mL; about 1,750 ng·hr/mL to about 2,000 ng·hr/mL; about 2,000 ng·hr/mL to about 2,500 ng·hr/mL; about 2,500 ng·hr/mL to about 3,000 ng·hr/mL; about 3,000 ng·hr/mL to about 3,500 ng·hr/mL; about 3,500 ng·hr/mL to about 4,000 ng·hr/mL; about 4,000 ng·hr/mL to about 4,500 ng·hr/mL; about 4,500 ng·hr/mL to about 5,000 ng·hr/mL; about 5,000 ng·hr/mL to about 5,500 ng·hr/mL; about 5,500 ng·hr/mL to about 6,000 ng·hr/mL; about 6,000 ng·hr/mL to about 6,500 ng·hr/mL; about 6,500 ng·hr/mL to about 7,000 ng·hr/mL; about 7,000 ng·hr/mL to about 7,500 ng·hr/mL; about 7,500 ng·hr/mL to about 8,000 ng·hr/mL; about 8,000 ng·hr/mL to about 8,500 ng·hr/mL; about 8,500 ng·hr/mL to about 9,000 ng·hr/mL; about 9,000 ng·hr/mL to about 9,500 ng·hr/mL; about 9,500 ng·hr/mL to about 10,000 ng·hr/mL; about 10,000 ng·hr/mL to about 10,500 ng·hr/mL; about 10,500 ng·hr/mL to about 11,000 ng·hr/mL; about 11,000 ng·hr/mL to about 11,500 ng·hr/mL; about 11,500 ng·hr/mL to about 12,000 ng·hr/mL; about 12,000 ng·hr/mL to about 12,500 ng·hr/mL; about 12,500 ng·hr/mL to about 13,000 ng·hr/mL; about 13,000 ng·hr/mL to about 13,500 ng·hr/mL; about 13,500 ng·hr/mL to about 14,000 ng·hr/mL; about 14,000 ng·hr/mL to about 14,500 ng·hr/mL; about 14,500 ng·hr/mL to about 15,000 ng·hr/mL; about 15,000 ng·hr/mL to about 15,500 ng·hr/mL; about 15,500 ng·hr/mL to about 16,000 ng·hr/mL; about 16,000 ng·hr/mL to about 16,500 ng·hr/mL; about 16,500 ng·hr/mL to about 17,000 ng·hr/mL; about 17,000 ng·hr/mL to about 17,500 ng·hr/mL; about 17,500 ng·hr/mL to about 18,000 ng·hr/mL; about 18,000 ng·hr/mL to about 18,500 ng·hr/mL; about 18,500 ng·hr/mL to about 19,000 ng·hr/mL; about 19,000 ng·hr/mL to about 19,500 ng·hr/mL; or about 19,500 ng·hr/mL to about 20,000 ng·hr/mL. For example, the AUC_{(0-inf)} of a compound can be about 8500 ng·hr/mL when administered intravenously at 50 μg/kg or about 4000 ng·hr/mL when administered subcutaneously at 50 μg/kg.

The plasma concentration of a compound described herein can be, for example, not less than about 1 ng/mL, not less than about 5 ng/mL, not less than about 10 ng/mL, not less than about 15 ng/mL, not less than about 20 ng/mL, not less than about 25 ng/mL, not less than about 50 ng/mL, not less than about 75 ng/mL, not less than about 100 ng/mL, not less than about 150 ng/mL, not less than about 200 ng/mL, not less than about 300 ng/mL, not less than about 400 ng/mL, not less than about 500 ng/mL, not less than about 600 ng/mL, not less than about 700 ng/mL, not less than about 800 ng/mL, not less than about 900 ng/mL, not less than about 1000 ng/mL, not less than about 1200 ng/mL, or any other plasma concentration of a compound described herein. The plasma concentration can be, for example, about 1 ng/mL to about 2,000 ng/mL; about 1 ng/mL to about 5 ng/mL; about 5 ng/mL to about 10 ng/mL; about 10 ng/mL to about 25 ng/mL; about 25 ng/mL to about 50 ng/mL; about 50 ng/mL to about 75 ng/mL; about 75 ng/mL to about 100 ng/mL; about 100 ng/mL to about 150 ng/mL; about 150 ng/mL to about 200 ng/mL; about 200 ng/mL to about 250 ng/mL; about 250 ng/mL to about 300 ng/mL; about 300 ng/mL to about 350 ng/mL; about 350 ng/mL to about 400 ng/mL; about 400 ng/mL to about 450 ng/mL; about 450 ng/mL to about 500 ng/mL; about 500 ng/mL to about 600 ng/mL; about 600 ng/mL to about 700 ng/mL; about 700 ng/mL to about 800 ng/mL; about 800 ng/mL to about 900 ng/mL; about 900 ng/mL to about 1,000 ng/mL; about 1,000 ng/mL to about 1,100 ng/mL; about 1,100 ng/mL to about 1,200 ng/mL; about 1,200 ng/mL to about 1,300 ng/mL; about 1,300 ng/mL to about 1,400 ng/mL; about 1,400 ng/mL to about 1,500 ng/mL; about 1,500 ng/mL to about 1,600 ng/mL; about 1,600 ng/mL to about 1,700 ng/mL; about 1,700 ng/mL to about 1,800 ng/mL; about 1,800 ng/mL to about 1,900 ng/mL; or about 1,900 ng/mL to about 2,000 ng/mL.

The pharmacodynamic parameters can be any parameters suitable for describing compositions of the disclosure. For example, the pharmacodynamic profile can exhibit increased Treg cell counts for, for example, about 24 hours, about 48 hours, about 72 hours, or 1 week.

Non-limiting examples of pharmacodynamic and pharmacokinetic parameters that can be calculated for a compound that is administered with the methods of the invention include: a) the amount of drug administered, which can be represented as a dose D; b) the dosing interval, which can be represented as τ; c) the apparent volume in which a drug is distributed, which can be represented as a volume of distribution V_{d}, where V_{d}=D/C₀; d) the amount of drug in a given volume of plasma, which can be represented as concentration C₀ or Cₛₛ, where C₀ or Cₛₛ=D/Vd and can be represented as a mean plasma concentration over a plurality of samples; e) the half-life of a drug t_{1/2}, where t_{1/2}=ln(2)/kₑ; f) the rate at which a drug is removed from the body kₑ, where kₑ=ln(2)/t_{1/2}=CL/V_{d}; g) the rate of infusion required to balance the equation Kᵢₙ, where Kᵢₙ=Cₛₛ·CL; h) the integral of the concentration-time curve after administration of a single dose, which can be represented as AUC_{0-∞}, wherein ∫₀^{∞} C dt, or in steady-state, which can be represented as AUCτ_{, ss}, wherein ∫ₜ^{t+π} C dt; i) the volume of plasma cleared of the drug per unit time, which can be represented as CL (clearance), wherein CL=V_{d}·kₑ=D/AUC; j) the systemically available fraction of a drug, which can be represented as f, where

$$f = \frac{AUCpo \cdot Div}{AUCiv \cdot Dpo} ; k )$$

the peak plasma concentration of a drug after administration Cₘₐₓ; l) the time taken by a drug to reach Cₘₐₓ, tₘₐₓ; m) the lowest concentration that a drug reaches before the next dose is administered Cₘᵢₙ; and n) the peak trough fluctuation within one dosing interval at steady state, which can be represented as % PTF=100.

$$\frac{( {C}_{max , ss} - {C}_{min , ss} )}{{C}_{av , ss}} where {C}_{av , ss} = \frac{{AUC}_{τ , ss}}{τ} .$$

The compounds of the present disclosure can have high stability when administered to a subject. The administered compound can have a physiological half-life of greater than about 6 hrs, greater than about 7 hrs, greater than about 8 hrs, greater than about 9 hrs, greater than about 10 hrs, greater than about 11 hrs, greater than about 12 hrs, greater than about 13 hrs, greater than about 14 hrs, greater than about 15 hrs, greater than about 16 hrs, greater than about 17 hrs, greater than about 18 hrs, greater than about 19 hrs, greater than about 20 hrs, greater than about 21 hrs, greater than about 22 hrs, greater than about 23 hrs, greater than about 24 hrs, greater than about 25 hrs, greater than about 26 hrs, greater than about 27 hrs, greater than about 28 hrs, greater than about 29 hrs, greater than about 30 hrs, greater than about 31 hrs, greater than about 32 hrs, greater than about 33 hrs, greater than about 34 hrs, greater than about 35 hrs, greater than about 36 hrs, greater than about 37 hrs, greater than about 38 hrs, greater than about 39 hrs, greater than about 40 hrs, greater than about 41 hrs, greater than about 42 hrs, greater than about 43 hrs, greater than about 44 hrs, greater than about 45 hrs, greater than about 46 hrs, greater than about 47 hrs, greater than about 48 hrs, greater than about 49 hrs, greater than about 50 hrs, greater than about 51 hrs, greater than about 52 hrs, greater than about 53 hrs, greater than about 54 hrs, greater than about 55 hrs, greater than about 56 hrs, greater than about 57 hrs, greater than about 58 hrs, greater than about 59 hrs, greater than about 60 hrs, greater than about 61 hrs, greater than about 62 hrs, greater than about 63 hrs, or greater than about 64 hrs.

The half-life of a compound of the present disclosure can vary based on the dose administered. For example, the half-life of the compound when administered in a dose of 50 μg/kg can be shorter than the half-life of the same compound when administered at a dose of 100 μg/kg or 250 μg/kg. The half-life of the compound can vary based on the administration route used. The half-life of the compound can be longer if the compound is administered subcutaneously rather than intravenously. For example the half-life of a compound delivered subcutaneously can be between about 15 hrs and about 25 hrs, while the half-life of the compound delivered intravenously can be between about 5 and about 15 hrs. In some embodiments, the half-life of a compound when administered intravenously at 50 μg/kg is about 6 hrs to about 14 hrs, about 7 hrs to about 13 hours, about 8 hrs to about 12 hrs, or about 9 hrs to about 11 hrs. In some embodiments, the half-life of a compound when administered intravenously at 50 μg/kg is about 5 hrs, about 6 hrs, about 7 hrs, about 8 hrs, about 9 hrs, about 10 hrs, about 11 hrs, about 12 hrs, about 13 hrs, about 14 hrs, or about 15 hrs. In some embodiments, the half-life of a compound when administered subcutaneously at 50 μg/kg is about 15 hrs to about 27 hrs, about 16 hrs to about 26 hours, about 17 hrs to about 25 hrs, about 18 hrs to about 24 hrs, about 19 hrs to about 23 hrs, or about 20 hrs to about 22 hrs. In some embodiments, the half-life of a compound when administered subcutaneously at 50 μg/kg is about 10 hrs, about 11 hrs, about 12 hrs, about 13 hrs, about 14 hrs, about 15 hrs, about 16 hrs, about 17 hrs, about 18 hrs, about 19 hrs, about 21 hrs, about 22 hrs, about 23 hrs, about 24 hrs, about 25 hrs, about 26 hrs, about 27 hrs, about 28 hrs, about 29 hrs, or about 30 hrs. The clearance of the compound from the blood can be faster for a compound delivered intravenously than for a compound delivered subcutaneously.

### Production of Dimeric Proteins: Heterodimers and Homodimers

In some embodiments, a compound of the present disclosure is a heterodimer, for example, a heterodimer comprising an IL2R-binding moiety (e.g. IL-2 or an IL-2 variant) that is part of a first fusion protein and an ST2-binding moiety (e.g. an antibody that binds ST2, or an antigen-binding fragment thereof) that is part of a second fusion protein. In some embodiments, each of the first and second fusion proteins comprises an IgG Fc domain, for example an IgG1 Fc domain or variant thereof. Heterodimers can be produced by expressing the two constituent recombinant proteins individually, purifying them, and combining them in vitro to form disulfide-linked heterodimers. Heterodimeric Fc fusion proteins can also be made in a single cell transfected with two constituent cDNA constructs using the “knobs into holes” approach. By this strategy, mutations are introduced into the CH2-CH3 interface between the two Fc polypeptide chains that prevent the formation of homodimers, yet form complementary interfaces that promote the formation of heterodimers. In this manner, heterodimeric Fc fusion proteins can be formed within host cells expressing the recombinant proteins and secreted as heterodimeric proteins. Below are two examples such Fc constructs on a human IgG1 background. The mutated residues T366Y and Y407T are shown in bold and underlined, and the N297A residue is underlined.

(A) IgG1 Fc (N297A; T366Y):
(SEQ ID NO: 8)
DKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCK
VSNKALPAPIEKTISKAKGQPREPQVYTLPPSRDELTKNQVSLYCLVKGFY
PSDIAVEWESNGQPENNYKTTPPVLDSDGSFFLYSKLTVDKSRWQQGNVFS
CSVMHEALHNHYTQKSLSLSPG
(B) IgG1 Fc (N297A; Y407T):
(SEQ ID NO: 9)
DKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLDSDGSFFLTSKLTVDKSRWQQGNVFS
CSVMHEALHNHYTQKSLSLSPG

The two different binding moieties, for instance, IL-2 and an ST2 antibody or fragment thereof, can be appended to the Fc sequences (A) and (B), respectively, to construct a heterodimeric protein.

In some embodiments, the first fusion protein comprises an IgG1 Fc domain comprising the mutations T350V, L351Y, F405A and Y407V (e.g. SEQ ID NO: 4); and the second fusion protein comprises the mutations T350V, T366L, K392L and T394W (e.g. SEQ ID NO: 5). Such mutations have been reported to improve proper pairing and stability (Von Kreudenstein et al., 2013, mAbs 5: 646-654; WO 2014082179 A1). Exemplary human IgG1 Fc domain sequences are shown below with the N297A mutation underlined, and the T350V, L351Y, F405A, Y407V, T350V, T366L, K392L and T394W mutations shown in bold and underlined.

(A) IgG1 Fc (N297A, T350V, L351Y, F405A and Y407V)
(SEQ ID NO: 4)
DKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCK
VSNKALPAPIEKTISKAKGQPREPQVYVYPPSRDELTKNQVSLTCLVKGFY
PSDIAVEWESNGQPENNYKTTPPVLDSDGSFALVSKLTVDKSRWQQGNVFS
CSVMHEALHNHYTQKSLSLSPG
(B) IgG1 Fc (N297A, T350V, T366L, K392L and T394W)
(SEQ ID NO: 5)
DKTHTCPPCPAPELLGGPSVFLFPPKPKDTLMISRTPEVTCVVVDVSHEDP
EVKFNWYVDGVEVHNAKTKPREEQYASTYRVVSVLTVLHQDWLNGKEYKCK
VSNKALPAPIEKTISKAKGQPREPQVYVLPPSRDELTKNQVSLLCLVKGFY
PSDIAVEWESNGQPENNYLTWPPVLDSDGSFFLYSKLTVDKSRWQQGNVFS
CSVMHEALHNHYTQKSLSLSPG

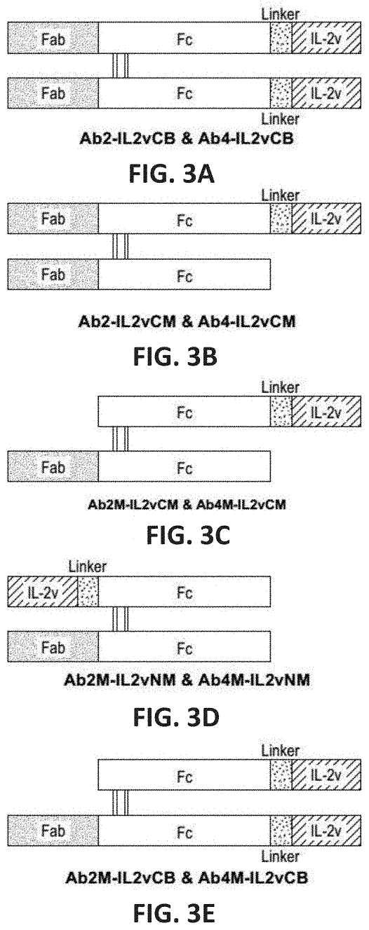

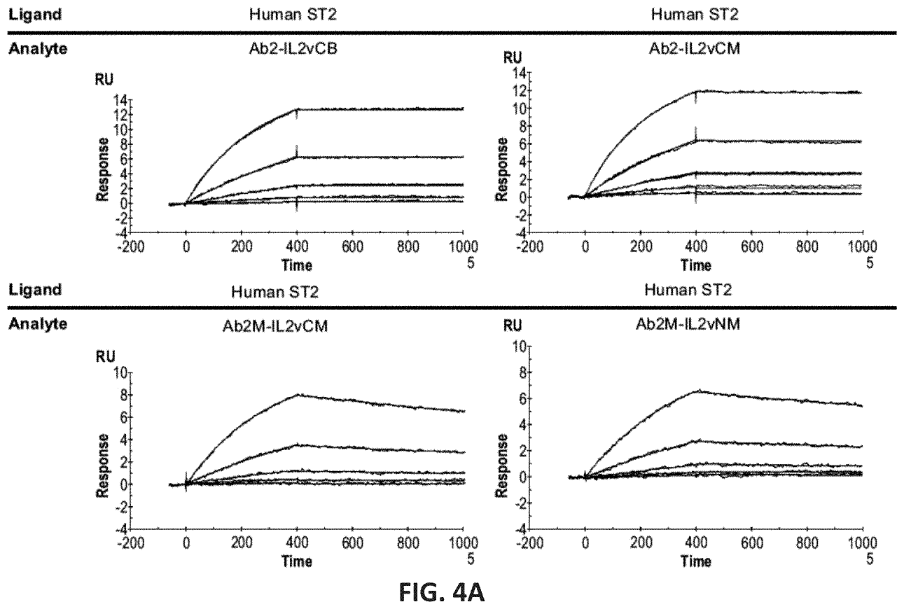

Exemplary heterodimers are shown in FIGS. 3A, 3B, 3C, 3D and 3E.

In some embodiments, a compound of the present disclosure is a homodimer, for example, a homodimer comprising two identical fusion proteins, each containing an IL2R-binding moiety (e.g. IL-2 or an IL-2 variant) and an ST2-binding moiety (e.g. an antibody that binds ST2, or an antigen-binding fragment thereof). In some embodiments, each of the two identical fusion proteins comprises an IgG Fc domain, for example an IgG1 Fc domain or variant thereof. In a particular embodiment, the IgG1 Fc domain is a human IgG1 Fc domain comprising the N297A mutation (e.g. SEQ ID NO: 7). Exemplary homodimers are shown in FIG. 3A.

The IL2R-binding moiety (e.g. IL-2 or an IL-2 variant) and the ST2-binding moiety (e.g. an antibody that binds ST2, or an antigen-binding fragment thereof) may be attached to the N-terminus or the C-terminus of the IgG Fe domain, either directly or through a peptide linker (e.g. a G₄S linker). Various combinations of an IL2R-binding moiety and an ST2-binding moiety may be used. In some embodiments, the dimeric protein comprises a first fusion protein comprising an IL2R-binding moiety N-terminal to the IgG Fc domain, and a second fusion protein comprising an ST2-binding moiety C-terminal to the IgG Fc domain. In some embodiments, the first fusion protein comprises an IL2R-binding moiety N-terminal to the IgG Fc domain, and the second fusion protein comprises an ST2-binding moiety N-terminal to the IgG Fc domain (see, for example, FIG. 3D). In some embodiments, the first fusion protein comprises an IL2R-binding moiety C-terminal to the IgG Fc domain, and the second fusion protein comprises an ST2-binding moiety N-terminal to the IgG Fc domain (see, for example, FIG. 3C). In some embodiments, the first fusion protein comprises an IL2R-binding moiety C-terminal to the IgG Fc domain, and the second fusion protein comprises an ST2-binding moiety C-terminal to the IgG Fc domain. In some embodiments, the first fusion protein comprises an ST2-binding moiety N terminal to the IgG Fc domain and an IL2R-binding moiety C-terminal to the IgG Fc domain, and the second fusion protein comprises an ST2-binding moiety N-terminal to the IgG Fc domain (see, for example, FIG. 3B). In some embodiments, the first fusion protein comprises an ST2-binding moiety N terminal to the IgG Fc domain and an IL2R-binding moiety C-terminal to the IgG Fc domain, and the second fusion protein comprises an IL2R-binding moiety N-terminal to the IgG Fc domain. In some embodiments, both the first fusion protein and the second fusion protein comprise an ST2-binding moiety N terminal to the IgG Fc domain and an IL2R-binding moiety C-terminal to the IgG Fc domain (see, for example, FIG. 3A). In some embodiments, both the first fusion protein and the second fusion protein comprise an IL2R-binding moiety N terminal to the IgG Fc domain and an ST2-binding moiety C-terminal to the IgG Fc domain. In some embodiments, the first fusion protein comprises an IL2R-binding moiety C-terminal to the IgG Fc domain, and the second fusion protein comprises an ST2-binding moiety N-terminal to the IgG Fc domain and an IL2R-binding moiety C-terminal to the IgG Fc domain (see, for example, FIG. 3E).

In some embodiments, the dimeric protein comprises at least one IL2R-binding moiety (e.g. IL-2 or an IL-2 variant) and at least one ST2-binding moiety (e.g. an antibody that binds ST2, or an antigen-binding fragment thereof). In some embodiments, the dimeric protein comprises only one IL2R-binding moiety. In some embodiments, the dimeric protein comprises only one ST2-binding moiety.

In some embodiments, the dimeric protein comprises at least two IL2R-binding moieties. For example, in some embodiments, the first and second fusion protein each contain at least one IL2R-binding moiety. See, for example, FIGS. 3A and 3E. In some embodiments, the dimeric protein comprises at least two ST2-binding moieties. For example, in some embodiments, the first and second fusion protein each contain at least one ST2-binding moiety. See, for example, FIGS. 3A and 3B.

In any of the embodiments described herein, the IL2R-binding moiety and the ST2-binding moiety may be attached to the IgG Fc domain via a peptide linker (e.g. a G₄S linker). The dimeric protein may contain, 1, 2, 3, 4 or more peptide linkers. In some embodiments, the dimeric protein comprises at least 1, 2, 3 or 4 peptide linkers.

In some embodiments, the IL2R-binding moiety and/or the ST2-binding moiety is fused directly to the IgG Fc domain through a peptide bond, i.e. without the addition of a peptide linker between the binding moiety and the IgG Fc domain. In some embodiment, the dimeric protein does not comprise a peptide linker.

In some embodiments, the first fusion protein of the dimeric protein is configured so that the C-terminus of the IL-2 binding moiety (e.g. a human IL-2 protein domain or a variant thereof) is fused through a peptide bond to the N-terminus of a first peptide linker domain; and the N-terminus of the first IgG Fc protein domain is fused through a peptide bond to the C-terminus of the first peptide linker domain. In some embodiments, the second fusion protein of the dimeric protein is configured so that the C-terminus of a second IgG Fc protein domain is fused through a peptide bond to the N-terminus of a second peptide linker domain; and the N-terminus of a protein domain that binds to ST2 is fused through a peptide bond to the C-terminus of the second peptide linker domain. In some embodiments, the second fusion protein of the dimeric protein is configured so that the C-terminus of the ST2 binding moiety is fused through a peptide bond to the N-terminus of a second peptide linker domain; and the N-terminus of the second IgG Fc protein domain is fused through a peptide bond to the C-terminus of the second peptide linker domain. In some embodiments, the dimeric protein comprises a fusion protein having at least 90%, at least 95%, at least 96%, at least 97%, at least 98%, or at least 99% sequence identity to the amino acid sequence of SEQ ID NO: 10. In some embodiments, the dimeric protein comprises a fusion protein having at least 90%, at least 95%, at least 96%, at least 97%, at least 98%, or at least 99% sequence identity to the amino acid sequence of SEQ ID NO: 11. In some embodiments, the dimeric protein comprises a fusion protein having at least 90%, at least 95%, at least 96%, at least 97%, at least 98%, or at least 99% sequence identity to the amino acid sequence of SEQ ID NO: 12. In some embodiments, the dimeric protein comprises a fusion protein having at least 90%, at least 95%, at least 96%, at least 97%, at least 98%, or at least 99% sequence identity to the amino acid sequence of SEQ ID NO: 13. In some embodiments, the dimeric protein comprises a fusion protein having at least 90%, at least 95%, at least 96%, at least 97%, at least 98%, or at least 99% sequence identity to the amino acid sequence of SEQ ID NO: 14. In some embodiments, the dimeric protein comprises a fusion protein having at least 90%, at least 95%, at least 96%, at least 97%, at least 98%, or at least 99% sequence identity to the amino acid sequence of SEQ ID NO: 15. In some embodiments, the dimeric protein comprises a fusion protein having at least 90%, at least 95%, at least 96%, at least 97%, at least 98%, or at least 99% sequence identity to the amino acid sequence of SEQ ID NO: 18.

In some embodiments, the dimeric protein further comprises light chains of an ST2 antibody. For example, in some embodiments, the fusion protein comprises a heavy chain of an ST2 antibody (e.g. a human IgG1 ST2 antibody heavy chain listed in Table 1), and the dimeric protein further comprises a corresponding light chain (e.g. a light chain listed in Table 1) of the ST2 antibody. The light chain may be linked to the heavy chain by a disulfide bond. In some embodiments, the dimeric protein comprises only one ST2 antibody, e.g. an antigen binding fragment (Fab) comprising one heavy chain and one light chain. In some embodiments, the dimeric protein comprises two ST2 antibodies, e.g. two Fab fragments each comprising a heavy chain and a light chain.

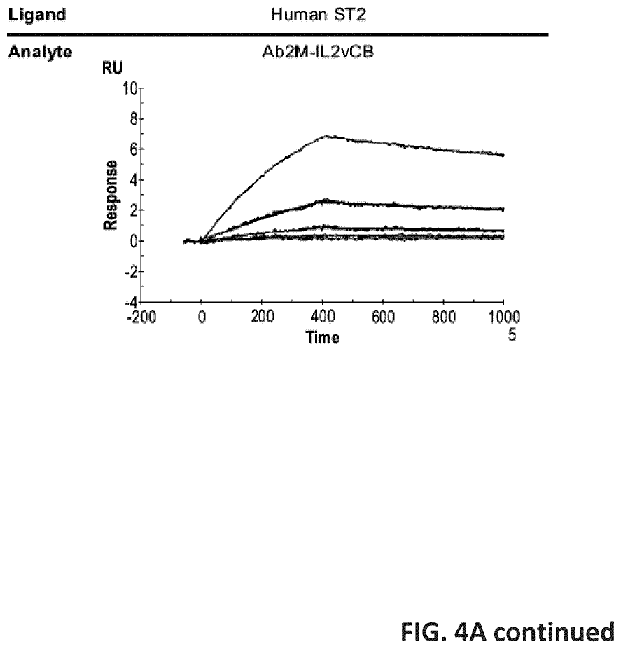

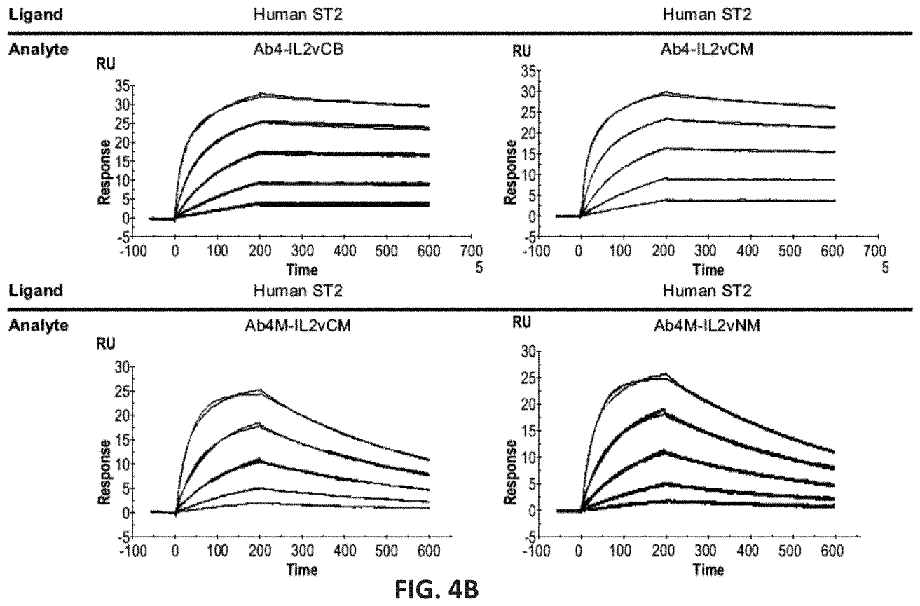

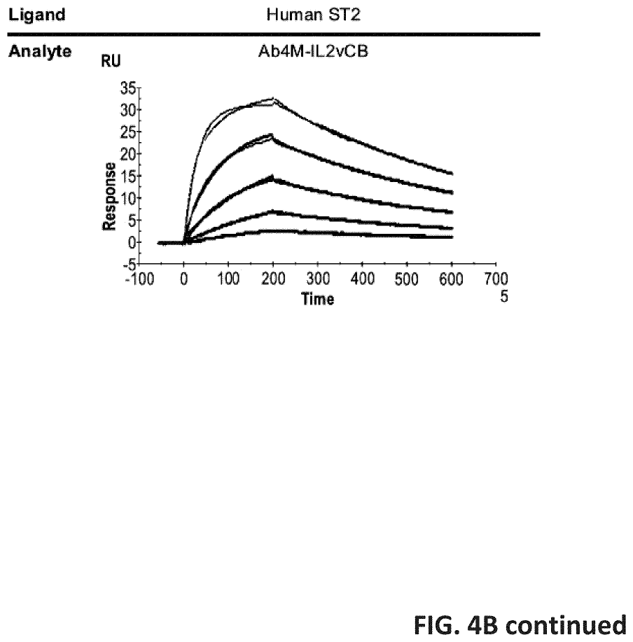

Description of Sequences in Sequence Listing

| SEQ | |
|---|---|
| ID NO: | Description |
| 1 | Human IL-2 comprising the N88R and C125S mutations |
| 2 | Wildtype human IL-2 |
| 3 | Human IL-2 comprising the T3A, N88R, and C125S |
| | mutations |
| 4 | Human IgG1 Fc (N297A, T350V, L351Y, F405A, Y407V) |
| 5 | Human IgG1 Fc (N297A, T350V, T366L, K392L, T394W) |
| 6 | peptide linker GGGGSGGGGSGGGGS (G4S)₃ |
| 7 | Human IgG1 Fc moiety (N297A) |
| 8 | Human IgG1 Fc (T366Y) |
| 9 | Human IgG1 Fc (Y407T) |
| 10 | IL-2 (N88R, C125)/(G4S)₃ linker/IgG1 Fc (N297A, T350V, |
| | T366L, K392L, T394W) fusion protein FIG. 4B |
| 11 | IgG1 Fc (N297A, T350V, T366L, K392L, T394W)/(G4S)₃ |
| | linker/IL-2 (N88R, C125) fusion protein FIG. 4C |
| 12 | Ab2HeavyIL2vC |
| 13 | Ab4HeavyIL2vC |
| 14 | Ab2HeavyIL2vC(W) |
| 15 | Ab4HeavyIL2vC(W) |
| 16 | Ab2Heavy(V) |
| 17 | Ab4Heavy(V) |
| 18 | IL2vCFc(V) |
| 19 | Ab2Kappa |
| 20 | Ab4Kappa |
| 21 | GGGGS peptide linker |
| 22 | ST2 Antibody 1 heavy chain nucleic acid |
| 23 | ST2 Antibody 1 heavy chain amino acid |
| 24 | ST2 Antibody 2 heavy chain nucleic acid |
| 25 | ST2 Antibody 2 heavy chain amino acid |
| 26 | ST2 Antibody 3 heavy chain nucleic acid |
| 27 | ST2 Antibody 3 heavy chain amino acid |
| 28 | ST2 Antibody 4 heavy chain nucleic acid |
| 29 | ST2 Antibody 4 heavy chain amino acid |
| 30 | ST2 Antibody 5 heavy chain nucleic acid |
| 31 | ST2 Antibody 5 heavy chain amino acid |
| 32 | ST2 Antibody 6 heavy chain nucleic acid |
| 33 | ST2 Antibody 6 heavy chain amino acid |
| 34 | ST2 Antibody 7 heavy chain nucleic acid |
| 35 | ST2 Antibody 7 heavy chain amino acid |
| 36 | ST2 Antibody 8 heavy chain nucleic acid |
| 37 | ST2 Antibody 8 heavy chain amino acid |
| 38 | ST2 Antibody 9 heavy chain nucleic acid |
| 39 | ST2 Antibody 9 heavy chain amino acid |
| 40 | ST2 Antibody 10 heavy chain nucleic acid |
| 41 | ST2 Antibody 10 heavy chain amino acid |
| 42 | ST2 Antibody 11 heavy chain nucleic acid |
| 43 | ST2 Antibody 11 heavy chain amino acid |
| 44 | ST2 Antibody 12 heavy chain nucleic acid |
| 45 | ST2 Antibody 12 heavy chain amino acid |
| 46 | ST2 Antibody 13 heavy chain nucleic acid |
| 47 | ST2 Antibody 13 heavy chain amino acid |
| 48 | ST2 Antibody 14 heavy chain nucleic acid |
| 48 | ST2 Antibody 14 heavy chain amino acid |
| 50 | ST2 Antibody 15 heavy chain nucleic acid |
| 51 | ST2 Antibody 15 heavy chain amino acid |
| 52 | ST2 Antibody 16 heavy chain nucleic acid |
| 53 | ST2 Antibody 16 heavy chain amino acid |
| 54 | ST2 Antibody 17 heavy chain nucleic acid |
| 55 | ST2 Antibody 17 heavy chain amino acid |
| 56 | ST2 Antibody 18 heavy chain nucleic acid |
| 57 | ST2 Antibody 18 heavy chain amino acid |
| 58 | ST2 Antibody 19 heavy chain nucleic acid |
| 59 | ST2 Antibody 19 heavy chain amino acid |
| 60 | ST2 Antibody 20 heavy chain nucleic acid |
| 61 | ST2 Antibody 20 heavy chain amino acid |
| 62 | ST2 Antibody 21 heavy chain nucleic acid |
| 63 | ST2 Antibody 21 heavy chain amino acid |
| 64 | ST2 Antibody 22 heavy chain nucleic acid |
| 65 | ST2 Antibody 22 heavy chain amino acid |
| 66 | ST2 Antibody 23 heavy chain nucleic acid |
| 67 | ST2 Antibody 23 heavy chain amino acid |
| 68 | ST2 Antibody 24 heavy chain nucleic acid |
| 69 | ST2 Antibody 24 heavy chain amino acid |
| 70 | ST2 Antibody 25heavy chain nucleic acid |
| 71 | ST2 Antibody 25 heavy chain amino acid |
| 72 | ST2 Antibody 26 heavy chain nucleic acid |
| 73 | ST2 Antibody 26 heavy chain amino acid |
| 74 | ST2 Antibody 27 heavy chain nucleic acid |
| 75 | ST2 Antibody 27 heavy chain amino acid |
| 76 | ST2 Antibody 28 heavy chain nucleic acid |
| 77 | ST2 Antibody 28 heavy chain amino acid |
| 78 | ST2 Antibody 29 heavy chain nucleic acid |
| 79 | ST2 Antibody 29 heavy chain amino acid |
| 80 | ST2 Antibody 30 heavy chain nucleic acid |
| 81 | ST2 Antibody 30 heavy chain amino acid |
| 82 | ST2 Antibody 31 heavy chain nucleic acid |
| 83 | ST2 Antibody 31 heavy chain amino acid |
| 84 | ST2 Antibody 32 heavy chain nucleic acid |
| 85 | ST2 Antibody 32 heavy chain amino acid |
| 86 | ST2 Antibody 33 heavy chain nucleic acid |
| 87 | ST2 Antibody 33 heavy chain amino acid |
| 88 | ST2 Antibody34 heavy chain nucleic acid |
| 89 | ST2 Antibody 34 heavy chain amino acid |
| 90 | ST2 Antibody 35 heavy chain nucleic acid |
| 91 | ST2 Antibody 35 heavy chain amino acid |
| 92 | ST2 Antibody 36 heavy chain nucleic acid |
| 93 | ST2 Antibody 36 heavy chain amino acid |
| 94 | ST2 Antibody 37 heavy chain nucleic acid |
| 95 | ST2 Antibody 37 heavy chain amino acid |
| 96 | ST2 Antibody 38 heavy chain nucleic acid |
| 97 | ST2 Antibody 38 heavy chain amino acid |
| 98 | ST2 Antibody 39 heavy chain nucleic acid |
| 99 | ST2 Antibody 39 heavy chain amino acid |
| 100 | ST2 Antibody 40 heavy chain nucleic acid |
| 101 | ST2 Antibody 40 heavy chain amino acid |
| 102 | ST2 Antibody 41 heavy chain nucleic acid |
| 103 | ST2 Antibody 41 heavy chain amino acid |
| 104 | ST2 Antibody 42 heavy chain nucleic acid |
| 105 | ST2 Antibody 42 heavy chain amino acid |
| 106 | ST2 Antibody 43 heavy chain nucleic acid |
| 107 | ST2 Antibody 43 heavy chain amino acid |
| 108 | ST2 Antibody 44 heavy chain nucleic acid |
| 109 | ST2 Antibody 44 heavy chain amino acid |
| 110 | ST2 Antibody 45 heavy chain nucleic acid |
| 111 | ST2 Antibody 45 heavy chain amino acid |
| 112 | ST2 Antibody 46 heavy chain nucleic acid |
| 113 | ST2 Antibody 46 heavy chain amino acid |
| 114 | ST2 Antibody 47 heavy chain nucleic acid |
| 115 | ST2 Antibody 47 heavy chain amino acid |
| 116 | ST2 Antibody 48 heavy chain nucleic acid |
| 117 | ST2 Antibody 48 heavy chain amino acid |
| 118 | ST2 Antibody 49 heavy chain nucleic acid |
| 119 | ST2 Antibody 49 heavy chain amino acid |
| 120 | ST2 Antibody 50 heavy chain nucleic acid |
| 121 | ST2 Antibody 50 heavy chain amino acid |
| 122 | ST2 Antibody 51 heavy chain nucleic acid |
| 123 | ST2 Antibody 51 heavy chain amino acid |
| 124 | ST2 Antibody 52 heavy chain nucleic acid |
| 125 | ST2 Antibody 52 heavy chain amino acid |
| 126 | ST2 Antibody 53 heavy chain nucleic acid |
| 127 | ST2 Antibody 53 heavy chain amino acid |
| 128 | ST2 Antibody 54 heavy chain nucleic acid |
| 129 | ST2 Antibody 54 heavy chain amino acid |
| 130 | ST2 Antibody 55 heavy chain nucleic acid |
| 131 | ST2 Antibody 55 heavy chain amino acid |
| 132 | ST2 Antibody 56 heavy chain nucleic acid |
| 133 | ST2 Antibody 56 heavy chain amino acid |
| 134 | ST2 Antibody 57 heavy chain nucleic acid |
| 135 | ST2 Antibody 57 heavy chain amino acid |
| 136 | ST2 Antibody 58 heavy chain nucleic acid |
| 137 | ST2 Antibody 58 heavy chain amino acid |
| 138 | ST2 Antibody 59 heavy chain nucleic acid |
| 139 | ST2 Antibody 59 heavy chain amino acid |
| 140 | ST2 Antibody 60 heavy chain nucleic acid |
| 141 | ST2 Antibody 60 heavy chain amino acid |
| 142 | ST2 Antibody 61 heavy chain nucleic acid |
| 143 | ST2 Antibody 61 heavy chain amino acid |
| 144 | ST2 Antibody 62 heavy chain nucleic acid |
| 145 | ST2 Antibody 62 heavy chain amino acid |
| 146 | ST2 Antibody 63 heavy chain nucleic acid |
| 147 | ST2 Antibody 63 heavy chain amino acid |
| 148 | ST2 Antibody 64 heavy chain nucleic acid |
| 149 | ST2 Antibody 64 heavy chain amino acid |
| 150 | ST2 Antibody 65 heavy chain nucleic acid |
| 151 | ST2 Antibody 65 heavy chain amino acid |
| 152 | ST2 Antibody 66 heavy chain nucleic acid |
| 153 | ST2 Antibody 66 heavy chain amino acid |
| 154 | ST2 Antibody 67 heavy chain nucleic acid |
| 155 | ST2 Antibody 67 heavy chain amino acid |
| 156 | ST2 Antibody 68 heavy chain nucleic acid |
| 157 | ST2 Antibody 68 heavy chain amino acid |
| 158 | ST2 Antibody 69 heavy chain nucleic acid |
| 159 | ST2 Antibody 69 heavy chain amino acid |
| 160 | ST2 Antibody 70 heavy chain nucleic acid |
| 161 | ST2 Antibody 70 heavy chain amino acid |
| 162 | ST2 Antibody 71 heavy chain nucleic acid |
| 163 | ST2 Antibody 71 heavy chain amino acid |
| 164 | ST2 Antibody 72 heavy chain nucleic acid |
| 165 | ST2 Antibody 72 heavy chain amino acid |
| 166 | ST2 Antibody 73 heavy chain nucleic acid |
| 167 | ST2 Antibody 73 heavy chain amino acid |
| 168 | ST2 Antibody 74 heavy chain nucleic acid |
| 169 | ST2 Antibody 74 heavy chain amino acid |
| 170 | ST2 Antibody 75 heavy chain nucleic acid |
| 171 | ST2 Antibody 75 heavy chain amino acid |
| 172 | ST2 Antibody 76 heavy chain nucleic acid |
| 173 | ST2 Antibody 76 heavy chain amino acid |
| 174 | ST2 Antibody 77 heavy chain nucleic acid |
| 175 | ST2 Antibody 77 heavy chain amino acid |
| 176 | ST2 Antibody 78 heavy chain nucleic acid |
| 177 | ST2 Antibody 78 heavy chain amino acid |
| 178 | ST2 Antibody 79 heavy chain nucleic acid |
| 179 | ST2 Antibody 79 heavy chain amino acid |
| 180 | ST2 Antibody 80 heavy chain nucleic acid |
| 181 | ST2 Antibody 80 heavy chain amino acid |
| 182 | ST2 Antibody 81 heavy chain nucleic acid |
| 183 | ST2 Antibody 81 heavy chain amino acid |
| 184 | ST2 Antibody 82 heavy chain nucleic acid |
| 185 | ST2 Antibody 82 heavy chain amino acid |
| 186 | ST2 Antibody 83 heavy chain nucleic acid |
| 187 | ST2 Antibody 83 heavy chain amino acid |
| 188 | ST2 Antibody 84 heavy chain nucleic acid |
| 189 | ST2 Antibody 84 heavy chain amino acid |
| 190 | ST2 Antibody 85 heavy chain nucleic acid |
| 191 | ST2 Antibody 85 heavy chain amino acid |
| 192 | ST2 Antibody 86 heavy chain nucleic acid |
| 193 | ST2 Antibody 86 heavy chain amino acid |
| 194 | ST2 Antibody 87 heavy chain nucleic acid |
| 195 | ST2 Antibody 87 heavy chain amino acid |
| 196 | ST2 Antibody 88 heavy chain nucleic acid |
| 197 | ST2 Antibody 88 heavy chain amino acid |
| 198 | ST2 Antibody 89 heavy chain nucleic acid |
| 199 | ST2 Antibody 89 heavy chain amino acid |
| 200 | ST2 Antibody 90 heavy chain nucleic acid |
| 201 | ST2 Antibody 90 heavy chain amino acid |
| 202 | ST2 Antibody 91 heavy chain nucleic acid |
| 203 | ST2 Antibody 91 heavy chain amino acid |
| 204 | ST2 Antibody 92 heavy chain nucleic acid |
| 205 | ST2 Antibody 92 heavy chain amino acid |
| 206 | ST2 Antibody 93 heavy chain nucleic acid |
| 207 | ST2 Antibody 93 heavy chain amino acid |
| 208 | ST2 Antibody 94 heavy chain nucleic acid |
| 209 | ST2 Antibody 94 heavy chain amino acid |
| 210 | ST2 Antibody 95 heavy chain nucleic acid |
| 211 | ST2 Antibody 95 heavy chain amino acid |
| 212 | ST2 Antibody 96 heavy chain nucleic acid |
| 213 | ST2 Antibody 96 heavy chain amino acid |
| 214 | ST2 Antibody 97 heavy chain nucleic acid |
| 215 | ST2 Antibody 97 heavy chain amino acid |
| 216 | ST2 Antibody 98 heavy chain nucleic acid |
| 217 | ST2 Antibody 98 heavy chain amino acid |
| 218 | ST2 Antibody 99 heavy chain nucleic acid |
| 219 | ST2 Antibody 99 heavy chain amino acid |
| 220 | ST2 Antibody 100 heavy chain nucleic acid |
| 221 | ST2 Antibody 100 heavy chain amino acid |
| 222 | ST2 Antibody 101 heavy chain nucleic acid |
| 223 | ST2 Antibody 101 heavy chain amino acid |
| 224 | ST2 Antibody 102 heavy chain nucleic acid |
| 225 | ST2 Antibody 102 heavy chain amino acid |
| 226 | ST2 Antibody 103 heavy chain nucleic acid |
| 227 | ST2 Antibody 103 heavy chain amino acid |
| 228 | ST2 Antibody 104 heavy chain nucleic acid |
| 229 | ST2 Antibody 104 heavy chain amino acid |
| 230 | ST2 Antibody 105 heavy chain nucleic acid |
| 231 | ST2 Antibody 105 heavy chain amino acid |
| 232 | ST2 Antibody 106 heavy chain nucleic acid |
| 233 | ST2 Antibody 106 heavy chain amino acid |
| 234 | ST2 Antibody 107 heavy chain nucleic acid |
| 235 | ST2 Antibody 107 heavy chain amino acid |
| 236 | ST2 Antibody 108 heavy chain nucleic acid |
| 237 | ST2 Antibody 108 heavy chain amino acid |
| 238 | ST2 Antibody 109 heavy chain nucleic acid |
| 239 | ST2 Antibody 109 heavy chain amino acid |
| 240 | ST2 Antibody 1 light chain nucleic acid |
| 241 | ST2 Antibody 1 light chain amino acid |
| 242 | ST2 Antibody 2 light chain nucleic acid |
| 243 | ST2 Antibody 2 light chain amino acid |
| 244 | ST2 Antibody 3 light chain nucleic acid |
| 245 | ST2 Antibody 3 light chain amino acid |
| 246 | ST2 Antibody 4 light chain nucleic acid |
| 247 | ST2 Antibody 4 light chain amino acid |
| 248 | ST2 Antibody 5 light chain nucleic acid |
| 249 | ST2 Antibody 5 light chain amino acid |
| 250 | ST2 Antibody 6 light chain nucleic acid |
| 251 | ST2 Antibody 6 light chain amino acid |
| 252 | ST2 Antibody 7 light chain nucleic acid |
| 253 | ST2 Antibody 7 light chain amino acid |
| 254 | ST2 Antibody 8 light chain nucleic acid |
| 255 | ST2 Antibody 8 light chain amino acid |
| 256 | ST2 Antibody 9 light chain nucleic acid |
| 257 | ST2 Antibody 9 light chain amino acid |
| 258 | ST2 Antibody 10 light chain nucleic acid |
| 259 | ST2 Antibody 10 light chain amino acid |
| 260 | ST2 Antibody 11 light chain nucleic acid |
| 261 | ST2 Antibody 11 light chain amino acid |
| 262 | ST2 Antibody 12 light chain nucleic acid |
| 263 | ST2 Antibody 12 light chain amino acid |
| 264 | ST2 Antibody 13 light chain nucleic acid |
| 265 | ST2 Antibody 13 light chain amino acid |
| 266 | ST2 Antibody 14 light chain nucleic acid |
| 267 | ST2 Antibody 14 light chain amino acid |
| 268 | ST2 Antibody 15 light chain nucleic acid |
| 269 | ST2 Antibody 15 light chain amino acid |
| 270 | ST2 Antibody 16 light chain nucleic acid |
| 271 | ST2 Antibody 16 light chain amino acid |
| 272 | ST2 Antibody 17 light chain nucleic acid |
| 273 | ST2 Antibody 17 light chain amino acid |
| 274 | ST2 Antibody 18 light chain nucleic acid |
| 275 | ST2 Antibody 18 light chain amino acid |
| 276 | ST2 Antibody 19 light chain nucleic acid |
| 277 | ST2 Antibody 19 light chain amino acid |
| 278 | ST2 Antibody 20 light chain nucleic acid |
| 279 | ST2 Antibody 20 light chain amino acid |
| 280 | ST2 Antibody 21 light chain nucleic acid |
| 281 | ST2 Antibody 21 light chain amino acid |
| 282 | ST2 Antibody 22 light chain nucleic acid |
| 283 | ST2 Antibody 22 light chain amino acid |
| 284 | ST2 Antibody 23 light chain nucleic acid |
| 285 | ST2 Antibody 23 light chain amino acid |
| 286 | ST2 Antibody 24 light chain nucleic acid |
| 287 | ST2 Antibody 24 light chain amino acid |
| 288 | ST2 Antibody 25 light chain nucleic acid |
| 289 | ST2 Antibody 25 light chain amino acid |
| 290 | ST2 Antibody 26 light chain nucleic acid |
| 291 | ST2 Antibody 26 light chain amino acid |
| 292 | ST2 Antibody 27 light chain nucleic acid |
| 293 | ST2 Antibody 27 light chain amino acid |
| 294 | ST2 Antibody 28 light chain nucleic acid |
| 295 | ST2 Antibody 28 light chain amino acid |
| 296 | ST2 Antibody 29 light chain nucleic acid |
| 297 | ST2 Antibody 29 light chain amino acid |
| 298 | ST2 Antibody 30 light chain nucleic acid |
| 299 | ST2 Antibody 30 light chain amino acid |
| 300 | ST2 Antibody 31 light chain nucleic acid |
| 301 | ST2 Antibody 31 light chain amino acid |
| 302 | ST2 Antibody 32 light chain nucleic acid |
| 303 | ST2 Antibody 32 light chain amino acid |
| 304 | ST2 Antibody 33 light chain nucleic acid |
| 305 | ST2 Antibody 33 light chain amino acid |
| 306 | ST2 Antibody34 light chain nucleic acid |
| 307 | ST2 Antibody 34 light chain amino acid |
| 308 | ST2 Antibody 35 light chain nucleic acid |
| 309 | ST2 Antibody 35 light chain amino acid |
| 310 | ST2 Antibody 36 light chain nucleic acid |
| 311 | ST2 Antibody 36 light chain amino acid |
| 312 | ST2 Antibody 37 light chain nucleic acid |
| 313 | ST2 Antibody 37 light chain amino acid |
| 314 | ST2 Antibody 38 light chain nucleic acid |
| 315 | ST2 Antibody 38 light chain amino acid |
| 316 | ST2 Antibody 39 light chain nucleic acid |
| 317 | ST2 Antibody 39 light chain amino acid |
| 318 | ST2 Antibody 40 light chain nucleic acid |
| 319 | ST2 Antibody 40 light chain amino acid |
| 320 | ST2 Antibody 41 light chain nucleic acid |
| 321 | ST2 Antibody 41 light chain amino acid |
| 322 | ST2 Antibody 42 light chain nucleic acid |
| 323 | ST2 Antibody 42 light chain amino acid |
| 324 | ST2 Antibody 43 light chain nucleic acid |
| 325 | ST2 Antibody 43 light chain amino acid |
| 326 | ST2 Antibody 44 light chain nucleic acid |
| 327 | ST2 Antibody 44 light chain amino acid |
| 328 | ST2 Antibody 45 light chain nucleic acid |
| 329 | ST2 Antibody 45 light chain amino acid |
| 330 | ST2 Antibody 46 light chain nucleic acid |
| 331 | ST2 Antibody 46 light chain amino acid |
| 332 | ST2 Antibody 47 light chain nucleic acid |
| 333 | ST2 Antibody 47 light chain amino acid |
| 334 | ST2 Antibody 48 light chain nucleic acid |
| 335 | ST2 Antibody 48 light chain amino acid |
| 336 | ST2 Antibody 49 light chain nucleic acid |
| 337 | ST2 Antibody 49 light chain amino acid |
| 338 | ST2 Antibody 50 light chain nucleic acid |
| 339 | ST2 Antibody 50 light chain amino acid |
| 340 | ST2 Antibody 51 light chain nucleic acid |
| 341 | ST2 Antibody 51 light chain amino acid |
| 342 | ST2 Antibody 52 light chain nucleic acid |
| 343 | ST2 Antibody 52 light chain amino acid |
| 344 | ST2 Antibody 53 light chain nucleic acid |
| 345 | ST2 Antibody 53 light chain amino acid |
| 346 | ST2 Antibody 54 light chain nucleic acid |
| 347 | ST2 Antibody 54 light chain amino acid |
| 348 | ST2 Antibody 55 light chain nucleic acid |
| 349 | ST2 Antibody 55 light chain amino acid |
| 350 | ST2 Antibody 56 light chain nucleic acid |
| 351 | ST2 Antibody 56 light chain amino acid |
| 352 | ST2 Antibody 57 light chain nucleic acid |
| 353 | ST2 Antibody 57 light chain amino acid |
| 354 | ST2 Antibody 58 light chain nucleic acid |
| 355 | ST2 Antibody 58 light chain amino acid |
| 356 | ST2 Antibody 59 light chain nucleic acid |
| 357 | ST2 Antibody 59 light chain amino acid |
| 358 | ST2 Antibody 60 light chain nucleic acid |
| 359 | ST2 Antibody 60 light chain amino acid |
| 360 | ST2 Antibody 61 light chain nucleic acid |
| 361 | ST2 Antibody 61 light chain amino acid |
| 362 | ST2 Antibody 62 light chain nucleic acid |
| 363 | ST2 Antibody 62 light chain amino acid |
| 364 | ST2 Antibody 63 light chain nucleic acid |
| 365 | ST2 Antibody 63 light chain amino acid |
| 366 | ST2 Antibody 64 light chain nucleic acid |
| 367 | ST2 Antibody 64 light chain amino acid |
| 368 | ST2 Antibody 65 light chain nucleic acid |
| 369 | ST2 Antibody 65 light chain amino acid |
| 370 | ST2 Antibody 66 light chain nucleic acid |
| 371 | ST2 Antibody 66 light chain amino acid |
| 372 | ST2 Antibody 67 light chain nucleic acid |
| 373 | ST2 Antibody 67 light chain amino acid |
| 374 | ST2 Antibody 68 light chain nucleic acid |
| 375 | ST2 Antibody 68 light chain amino acid |
| 376 | ST2 Antibody 69 light chain nucleic acid |
| 377 | ST2 Antibody 69 light chain amino acid |
| 378 | ST2 Antibody 70 light chain nucleic acid |
| 379 | ST2 Antibody 70 light chain amino acid |
| 380 | ST2 Antibody 71 light chain nucleic acid |
| 381 | ST2 Antibody 71 light chain amino acid |
| 382 | ST2 Antibody 72 light chain nucleic acid |
| 383 | ST2 Antibody 72 light chain amino acid |
| 384 | ST2 Antibody 73 light chain nucleic acid |
| 385 | ST2 Antibody 73 light chain amino acid |
| 386 | ST2 Antibody 74 light chain nucleic acid |
| 387 | ST2 Antibody 74 light chain amino acid |
| 388 | ST2 Antibody 75 light chain nucleic acid |
| 389 | ST2 Antibody 75 light chain amino acid |
| 390 | ST2 Antibody 76 light chain nucleic acid |
| 391 | ST2 Antibody 76 light chain amino acid |
| 392 | ST2 Antibody 77 light chain nucleic acid |
| 393 | ST2 Antibody 77 light chain amino acid |
| 394 | ST2 Antibody 78 light chain nucleic acid |
| 395 | ST2 Antibody 78 light chain amino acid |
| 396 | ST2 Antibody 79 light chain nucleic acid |
| 397 | ST2 Antibody 79 light chain amino acid |
| 398 | ST2 Antibody 80 light chain nucleic acid |
| 399 | ST2 Antibody 80 light chain amino acid |
| 400 | ST2 Antibody 81 light chain nucleic acid |
| 401 | ST2 Antibody 81 light chain amino acid |
| 402 | ST2 Antibody 82 light chain nucleic acid |
| 403 | ST2 Antibody 82 light chain amino acid |
| 404 | ST2 Antibody 83 light chain nucleic acid |
| 405 | ST2 Antibody 83 light chain amino acid |
| 406 | ST2 Antibody 84 light chain nucleic acid |
| 407 | ST2 Antibody 84 light chain amino acid |
| 408 | ST2 Antibody 85 light chain nucleic acid |
| 409 | ST2 Antibody 85 light chain amino acid |
| 410 | ST2 Antibody 86 light chain nucleic acid |
| 411 | ST2 Antibody 86 light chain amino acid |
| 412 | ST2 Antibody 87 light chain nucleic acid |
| 413 | ST2 Antibody 87 light chain amino acid |
| 414 | ST2 Antibody 88 light chain nucleic acid |
| 415 | ST2 Antibody 88 light chain amino acid |
| 416 | ST2 Antibody 89 light chain nucleic acid |
| 417 | ST2 Antibody 89 light chain amino acid |
| 418 | ST2 Antibody 90 light chain nucleic acid |
| 419 | ST2 Antibody 90 light chain amino acid |
| 420 | ST2 Antibody 91 light chain nucleic acid |
| 421 | ST2 Antibody 91 light chain amino acid |
| 422 | ST2 Antibody 92 light chain nucleic acid |
| 423 | ST2 Antibody 92 light chain amino acid |
| 424 | ST2 Antibody 93 light chain nucleic acid |
| 425 | ST2 Antibody 93 light chain amino acid |
| 426 | ST2 Antibody 94 light chain nucleic acid |
| 427 | ST2 Antibody 94 light chain amino acid |
| 428 | ST2 Antibody 95 light chain nucleic acid |
| 429 | ST2 Antibody 95 light chain amino acid |
| 430 | ST2 Antibody 96 light chain nucleic acid |
| 431 | ST2 Antibody 96 light chain amino acid |
| 432 | ST2 Antibody 97 light chain nucleic acid |
| 433 | ST2 Antibody 97 light chain amino acid |
| 434 | ST2 Antibody 98 light chain nucleic acid |
| 435 | ST2 Antibody 98 light chain amino acid |
| 436 | ST2 Antibody 99 light chain nucleic acid |
| 437 | ST2 Antibody 99 light chain amino acid |
| 438 | ST2 Antibody 100 light chain nucleic acid |
| 439 | ST2 Antibody 100 light chain amino acid |
| 440 | ST2 Antibody 101 light chain nucleic acid |
| 441 | ST2 Antibody 101 light chain amino acid |
| 442 | ST2 Antibody 102 light chain nucleic acid |
| 443 | ST2 Antibody 102 light chain amino acid |
| 444 | ST2 Antibody 103 light chain nucleic acid |
| 445 | ST2 Antibody 103 light chain amino acid |
| 446 | ST2 Antibody 104 light chain nucleic acid |
| 447 | ST2 Antibody 104 light chain amino acid |
| 448 | ST2 Antibody 105 light chain nucleic acid |
| 449 | ST2 Antibody 105 light chain amino acid |
| 450 | ST2 Antibody 106 light chain nucleic acid |
| 451 | ST2 Antibody 106 light chain amino acid |
| 452 | ST2 Antibody 107 light chain nucleic acid |
| 453 | ST2 Antibody 107 light chain amino acid |
| 454 | ST2 Antibody 108 light chain nucleic acid |
| 455 | ST2 Antibody 108 light chain amino acid |
| 456 | ST2 Antibody 109 light chain nucleic acid |
| 457 | ST2 Antibody 109 light chain amino acid |
| 458-1111 | CDR amino acid sequences of Antibodies 1-109, see Table 2D |

### EXAMPLES

### Example 1. Construction of ST2 and IL2R Targeting Bispecific Molecules

Bispecific molecules targeting ST2 (IL-33 receptor), and IL-2 high affinity receptor were constructed. All constructed molecules are listed in Table 1 below. Their schematic diagrams are shown in FIG. 3.

Bispecific molecules in Table 1 and FIG. 3 are comprised of the human IL-2 variant (N88R, C125S) and an antigen-binding fragment (Fab) of an anti-ST2 antibody. As a proof of concept, two anti-ST2 mAbs, Ab2 and Ab4, were selected from a published patent application (US2017/0002079 A1). Each bispecific molecule is either monovalent or bivalent with respect to the anti-ST2 antigen binding fragment and is covalently connected to the IL-2 receptor agonist at the N or C terminus through the peptide linker, (G₄S)₃.

Production of heterodimeric Fc proteins can be challenging due to potential homodimer contamination. All heterodimeric molecules in this example have mutations on the Fc domain to reduce unwanted homodimer pairing. The mutations include T350V, L351Y, F405A & Y407V on one chain; and T350V, T366L, K392L & T394W on the other chain. Such mutations have been reported to improve proper pairing and stability (Von Kreudenstein et al., 2013, mAbs 5: 646-654; WO 2014082179 A1).

TABLE 1
Dimeric proteins comprising Fc regions, an antigen-binding fragment
of an anti-ST2 antibody, and an IL-2 variant (N88R, C125S).
Diagrams of the proteins are provided in FIG. 3A-3E.

| | Dimeric | Heavy Chain 1 | Heavy Chain 2 | |
|---|---|---|---|---|
| FIG. | Protein | (Fusion Protein) | (Fusion Protein) | Light Chains |
| 3A | Ab2-IL2vCB | Ab2Heavy-IL2vC | (same as Heavy | Ab2Kappa |
| | | (SEQ ID NO: 12) | Chain 1, homodimer) | (SEQ ID NO: 19) |
| | Ab4-IL2vCB | Ab4Heavy-IL2vC | (same as Heavy | Ab4Kappa |
| | | (SEQ ID NO: 13) | Chain 1, homodimer) | (SEQ ID NO: 20) |
| 3B | Ab2-IL2vCM | Ab2Heavy(V) | Ab2HeavyIL2vC(W) | Ab2Kappa |
| | | (SEQ ID NO: 16) | (SEQ ID NO: 14) | (SEQ ID NO: 19) |
| | Ab4-IL2vCM | Ab4Heavy(V) | Ab4HeavyIL2vC(W) | Ab4Kappa |
| | | (SEQ ID NO: 17) | (SEQ ID NO: 15) | (SEQ ID NO: 20) |
| 3C | Ab2M-IL2vCM | Ab2Heavy(V) | IL2vCFc(W) | Ab2Kappa |
| | | (SEQ ID NO: 16) | (SEQ ID NO: 11) | (SEQ ID NO: 19) |
| | AB4M-IL2vCM | Ab4Heavy(V) | IL2vCFc(W) | Ab4Kappa |
| | | (SEQ ID NO: 17) | (SEQ ID NO: 11) | (SEQ ID NO: 20) |
| 3D | Ab2M-IL2vNM | Ab2Heavy(V) | IL2vNFc(W) | Ab2Kappa |
| | | (SEQ ID NO: 16) | (SEQ ID NO: 10) | (SEQ ID NO: 19) |
| | Ab4M-IL2vNM | Ab4Heavy(V) | IL2vNFc(W) | Ab4Kappa |
| | | (SEQ ID NO: 17) | (SEQ ID NO: 10) | (SEQ ID NO: 20) |
| 3E | Ab2M-IL2vCB | IL2vCFc(V) | Ab2HeavyIL2vC(W) | Ab2Kappa |
| | | (SEQ ID NO: 18) | (SEQ ID NO: 14) | (SEQ ID NO: 19) |
| | Ab4M-IL2vCB | IL2vCFc(V) | Ab4HeavyIL2C(W) | Ab4Kappa |
| | | (SEQ ID NO: 18) | (SEQ ID NO: 15) | (SEQ ID NO: 20) |

All molecules were produced in transiently-transfected HEK293 cells and purified by Protein A affinity chromatography followed by size exclusion chromatography.

### Example 2. Binding Characterization of Anti-ST2/IL2v Bispecific Molecules

Binding of anti-ST2/IL2v bispecific proteins to human ST2 and IL2Ra was evaluated by surface plasmon resonance (SPR) using the Biacore T200 instrument (GE). Anti-His Tag antibody (GenScript) was immobilized on CM4 chips (GE) by NHS-EDS coupling, and binding reactions were carried out in HBS-EP+ buffer (GE) at 25° C. His-tagged ST2 ECD protein was captured by anti-His Tag antibody coated chips.

For ST2 binding, histidine tagged human ST2 ECD protein was captured on the chip as ligand. Ab2-IL2v bispecific molecules, which are comprised of the Fab of anti-ST2 mAb (Ab2) and IL-2v, were injected as analytes at a flow rate of 50 μl/min for 400 sec and allowed to dissociated for 600 sec. Ab2-IL2v bispecific molecules were prepared at various concentrations (0.012 nM-1 nM by 3-fold dilution). Ab4-IL2v bispecific molecules, which are comprised of the Fab of anti-ST2 mAb, Ab4 and IL-2v, were injected as analyte at a flow rate of 50 μl/min for 200 sec and allowed to dissociated for 400 sec. Ab4-IL2v bispecific molecules were prepared at various concentrations (0.062 nM-5 nM by 3-fold dilution). The chip surface was regenerated with 10 mM glycine pH 1.7. Association and dissociation signals of monovalent anti-ST2 bispecific molecules were fitted to 1:1 binding, using Biacore Evaluation Software Version 2.0 to yield kinetic constants (kₐ & k_{d}) and to calculate the dissociation constants (K_{d}).

Sensorgrams are shown in FIGS. 4A and 4B; and kinetic constants and dissociation constants are summarized in Table 3 below. All bispecific molecules exhibited clear binding to ST2 protein. K_{d} values of monovalent Ab2-IL2v bispecific molecules ranged from 94 pM to 137 pM in comparison to the reported value, 34 pM in patent US2017/0002079 A1. K_{d} values of monovalent Ab4/IL2v bispecific molecules ranged from 289 pM to 378 pM in comparison to the reported value, 301 pM in patent US2017/0002079 A1.

TABLE 3
Analysis of binding to human ST2 ECD protein

| Analyte | Ligand | kₐ (1/Ms) | k_{d} (1/s) | K_{d} (M) |
|---|---|---|---|---|
| Ab2M-IL2vCM | Human ST2 | 3.28E6 | 3.47E−4 | 1.06E−10 |
| Ab2M-IL2vNM | Human ST2 | 3.34E6 | 3.17E−4 | 9.43E−11 |
| Ab2M-IL2vCB | Human ST2 | 2.50E6 | 3.44E−4 | 1.37E−10 |
| Ab4M-IL2vCM | Human ST2 | 5.57E6 | 0.0020 | 3.68E−10 |
| Ab4M-IL2vNM | Human ST2 | 5.44E6 | 0.0021 | 3.78E−10 |
| Ab4M-IL2vCB | Human ST2 | 6.16E6 | 0.0018 | 2.89E−10 |

To test if the bispecific molecules bind to both ST2 and IL2Ra simultaneously, the bispecific molecules were captured by histidine tagged human ST2, which was immobilized on the chip. Subsequently, IL2R alpha was injected as analyte at a flow rate of 50 μl/min for 50 sec and allowed to dissociated for 60 sec. IL2Ra was prepared at various concentrations (2.5 nM-200 nM by 3 fold dilution). The chip surface was regenerated with 10 mM glycine pH 1.7. Association and dissociation signals were fitted to 1:1 binding, using Biacore Evaluation Software Version 2.0 to yield kinetic constants (kₐ & k_{d}) and to calculate the dissociation constants (K_{d}).

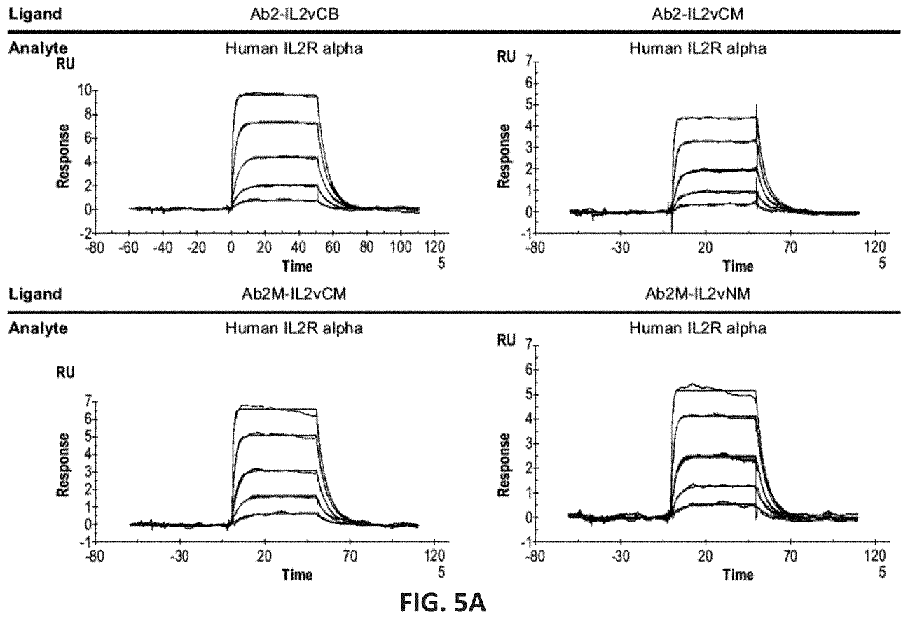

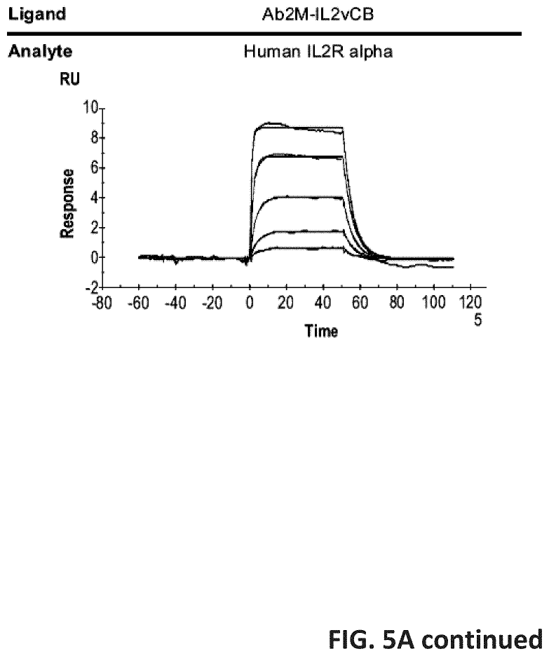

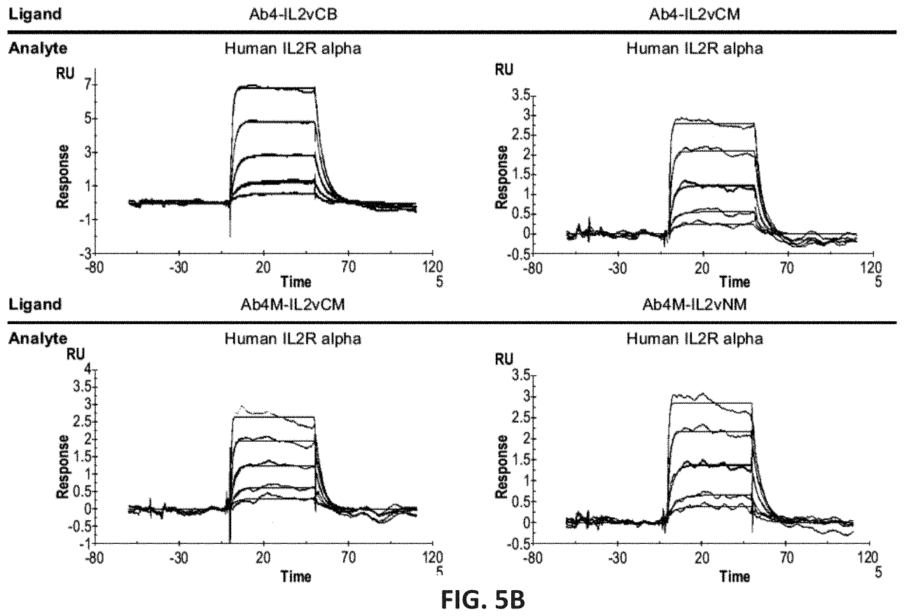

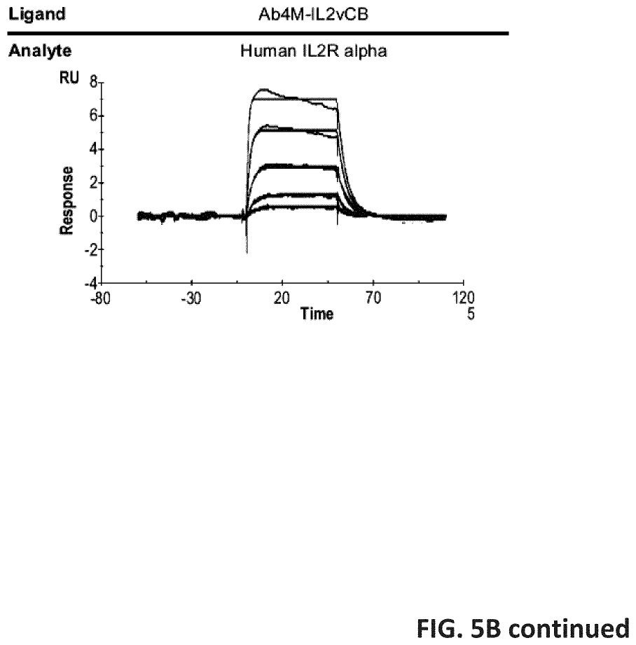

Sensorgrams are shown in FIGS. 5A and 5B; and kinetic constants and dissociation constants are summarized in Table 4 below. They all bound to human IL2R alpha at K_{d} values (25-43 nM) comparable to the previously reported values. This shows the IL2v moiety retains IL2Ra binding; and the bispecific molecules are able to bind to both ST2 and IL2Ra simultaneously because they showed binding to IL2Ra while being bound to ST2 protein.

TABLE 4
Simultaneous binding of proteins
to both ST2 ECD and IL2Ra ECD

| | ST2-bound | | | |
|---|---|---|---|---|
| Analyte | Ligand | kₐ (1/Ms) | k_{d} (1/s) | K_{d} (M) |
| Human IL2R alpha | Ab2M-IL2vCM | 6.73E6 | 0.1819 | 2.70E−8 |
| Human IL2R alpha | Ab2M-IL2vNM | 6.26E6 | 0.1550 | 2.48E−8 |
| Human IL2R alpha | Ab2M-IL2vCB | 5.78E6 | 0.1885 | 3.26E−8 |
| Human IL2R alpha | Ab4M-IL2vCM | 9.84E6 | 0.2513 | 2.55E−8 |
| Human IL2R alpha | Ab4M-IL2vNM | 7.14E6 | 0.1788 | 2.51E−8 |
| Human IL2R alpha | Ab4M-IL2vCB | 5.27E6 | 0.2002 | 3.80E−8 |
| Human IL2R alpha | Ab2-IL2vCB | 4.59E6 | 0.1633 | 3.56E−8 |
| Human IL2R alpha | Ab2-IL2vCM | 5.13E6 | 0.1677 | 3.27E−8 |
| Human IL2R alpha | Ab4-IL2vCB | 4.55E6 | 0.1944 | 4.27E−8 |
| Human IL2R alpha | Ab4-IL2vCM | 6.26E6 | 0.2568 | 4.10E−8 |

### Example 3. Isolation of Novel Human ST2 Antibodies

Novel human ST2 antibodies were generated by using a human scFv phage display library. Selection was performed with the recombinant human ST2 extracellular domain (ECD). Phage particles that were enriched after the 3 rounds of selection were subject to ELISA screening. The scFv fragments from the phage particles that showed binding to the ST2 ECD by ELISA were reformatted to IgG1.

### Example 4. Binding Affinity of Human ST2 Antibodies to Human ST2 and Mouse ST2

Binding of the reformatted human ST2 antibodies to the human and mouse ST2 proteins was evaluated by surface plasmon resonance (SPR), using a high throughput antibody screening and characterization platform from Carterra (Salt Lake City, Utah). The antibodies were immobilized as ligand molecules, and the ST2 proteins were flowed through as analyte molecules to obtain 1 tol binding kinectic rates and affinity, as shown in Table 5 below.

TABLE 5
Binding Affinity of human ST2 antibodies to human ST2 and mouse ST2.

| ST2 Antibody | Human ST2 | | | | | |
|---|---|---|---|---|---|---|
| # | ka (M−1 s−1) | kd (s−1) | KD (M) | | Rmax | Res sd |
| 1 | 1.200E+005 | 7.200E−002 | 617 | nM | 2.390E+002 | 8.000E+000 |
| 2 | 5.00E+005 | 1.10E−001 | 229 | nM | 279 | 8.3 |
| 3 | 2.90E+005 | 4.20E−002 | 146 | nM | 289 | 7.4 |
| 4 | 2.10E+005 | 2.50E−003 | 12 | nM | 274 | 12 |
| 5 | 2.50E+005 | 4.80E−002 | 190 | nM | 263 | 5 |
| 6 | 3.400E+005 | 5.700E−002 | 166 | nM | 2.310E+002 | 4.800E+000 |
| 7 | 2.90E+005 | 4.40E−003 | 15 | nM | 276 | 6.8 |
| 8 | 1.10E+005 | 1.70E−002 | 153 | nM | 288 | 4.4 |
| 9 | 1.20E+005 | 3.60E−002 | 293 | nM | 281 | 4.7 |
| 10 | 6.00E+005 | 7.90E−002 | 131 | nM | 273 | 9 |
| 11 | 7.80E+005 | 1.90E−002 | 25 | nM | 317 | 7.4 |
| 12 | 1.10E+005 | 2.40E−002 | 217 | nM | 311 | 5.3 |
| 13 | 2.00E+005 | 4.00E−002 | 201 | nM | 294 | 5.7 |
| 14 | 4.00E+005 | 1.70E−002 | 42 | nM | 252 | 6.3 |
| 15 | 1.90E+005 | 1.40E−001 | 769 | nM | 244 | 6.3 |
| 16 | 5.80E+005 | 5.60E−002 | 96 | nM | 290 | 6.6 |
| 17 | 7.60E+004 | 3.30E−002 | 433 | nM | 279 | 4.2 |
| 18 | 9.50E+004 | 1.10E−001 | 1.1 | uM | 217 | 3.9 |
| 19 | 5.50E+005 | 6.20E−002 | 113 | nM | 284 | 6.1 |
| 20 | 8.10E+005 | 6.50E−002 | 81 | nM | 277 | 7.4 |
| 21 | 2.10E+004 | 1.70E−002 | 805 | nM | 146 | 2.6 |
| 22 | 5.30E+005 | 4.80E−002 | 90 | nM | 240 | 4.4 |
| 23 | 1.50E+005 | 1.00E−001 | 688 | nM | 223 | 3.6 |
| 24 | 4.50E+004 | 5.50E−002 | 1.2 | uM | 166 | 4.5 |
| 25 | 1.50E+005 | 3.00E−002 | 193 | nM | 292 | 5.6 |
| 26 | 5.30E+005 | 1.40E−001 | 264 | nM | 273 | 9.5 |
| 27 | 4.50E+005 | 4.80E−002 | 106 | nM | 139 | 4.4 |
| 28 | 1.60E+005 | 6.60E−002 | 426 | nM | 258 | 6.4 |
| 29 | 1.40E+004 | 6.50E−003 | 480 | nM | 95 | 3.6 |
| 30 | 1.90E+005 | 2.90E−002 | 155 | nM | 264 | 4.3 |
| 31 | 4.30E+005 | 1.20E−001 | 274 | nM | 262 | 7.4 |
| 32 | 2.40E+005 | 6.10E−002 | 252 | nM | 262 | 4.6 |
| 33 | 2.40E+005 | 1.30E−001 | 529 | nM | 263 | 7.4 |
| 34 | 1.00E+004 | 1.10E−003 | 109 | nM | 165 | 8 |
| 35 | 1.60E+005 | 1.60E−002 | 104 | nM | 300 | 5.3 |
| 36 | 5.10E+005 | 1.40E−001 | 264 | nM | 297 | 4.2 |
| 37 | 6.20E+005 | 8.50E−002 | 138 | nM | 296 | 12 |
| 38 | 3.10E+005 | 5.30E−003 | 17 | nM | 248 | 5.4 |
| 39 | 3.90E+005 | 2.20E−002 | 57 | nM | 313 | 7 |
| 40 | 1.80E+005 | 1.20E−001 | 691 | nM | 245 | 6.5 |
| 41 | 2.40E+004 | 2.40E−003 | 99 | nM | 262 | 4.1 |
| 42 | 3.20E+005 | 8.80E−002 | 278 | nM | 242 | 8.1 |
| 43 | 5.10E+004 | 7.40E−003 | 146 | nM | 318 | 8.1 |
| 44 | 2.40E+005 | 2.40E−002 | 100 | nM | 290 | 6 |
| 45 | 9.20E+005 | 2.00E−002 | 22 | nM | 318 | 8.8 |
| 46 | 1.20E+005 | 4.20E−002 | 339 | nM | 263 | 5.5 |
| 47 | 3.00E+005 | 1.20E−001 | 413 | nM | 264 | 6.2 |
| 48 | 1.70E+005 | 2.40E−002 | 146 | nM | 317 | 4.1 |
| 49 | 3.20E+005 | 8.50E−002 | 269 | nM | 272 | 6.8 |
| 50 | 3.50E+005 | 7.50E−002 | 213 | nM | 278 | 7.2 |
| 51 | 2.80E+005 | 4.30E−002 | 154 | nM | 381 | 7.5 |
| 52 | 1.60E+005 | 1.50E−001 | 932 | nM | 190 | 4.2 |
| 53 | 1.60E+005 | 1.20E−001 | 740 | nM | 230 | 6.8 |
| 54 | 3.60E+005 | 2.90E−002 | 81 | nM | 301 | 8.7 |
| 55 | 3.00E+005 | 1.00E−001 | 349 | nM | 254 | 7.3 |
| 56 | 1.50E+005 | 1.40E−001 | 970 | nM | 204 | 5.2 |
| 57 | 5.10E+005 | 7.20E−002 | 140 | nM | 272 | 4.9 |
| 58 | 2.20E+005 | 1.40E−001 | 628 | nM | 259 | 6.3 |
| 59 | 1.90E+005 | 1.90E−001 | 1.0 | uM | 232 | 3.7 |
| 60 | 1.10E+005 | 1.20E−001 | 1.1 | uM | 163 | 4.7 |
| 61 | 2.60E+005 | 1.40E−001 | 563 | nM | 224 | 3.9 |
| 62 | 9.60E+005 | 6.80E−002 | 71 | nM | 236 | 6.7 |
| 63 | 4.50E+005 | 4.30E−002 | 96 | nM | 240 | 6.6 |
| 64 | 3.30E+005 | 1.20E−001 | 366 | nM | 263 | 6.2 |
| 65 | 7.30E+005 | 5.50E−002 | 76 | nM | 268 | 8.3 |
| 66 | 1.60E+004 | 2.80E−003 | 176 | nM | 32 | 2.8 |
| 67 | 1.40E+005 | 1.00E−001 | 738 | nM | 234 | 7 |
| 68 | 1.90E+005 | 1.30E−001 | 704 | nM | 205 | 3.4 |
| 69 | 4.10E+005 | 2.90E−002 | 71 | nM | 278 | 9.7 |
| 70 | 3.70E+005 | 9.90E−002 | 268 | nM | 281 | 10 |
| 71 | 1.40E+005 | 1.20E−001 | 912 | nM | 231 | 5.4 |
| 72 | 2.40E+005 | 8.20E−002 | 335 | nM | 233 | 5.3 |
| 73 | 1.40E+005 | 8.50E−002 | 603 | nM | 262 | 3.6 |
| 74 | 2.00E+005 | 1.20E−002 | 60 | nM | 297 | 7.4 |
| 75 | 1.20E+005 | 4.10E−002 | 360 | nM | 234 | 5.3 |
| 76 | 8.20E+005 | 1.10E−001 | 133 | nM | 301 | 7.3 |
| 77 | 2.60E+005 | 9.90E−002 | 377 | nM | 254 | 7.2 |
| 78 | 5.30E+005 | 8.20E−002 | 154 | nM | 195 | 4.7 |
| 79 | N/A | N/A | N/A | | N/A | N/A |
| 80 | 6.70E+004 | 5.60E−002 | 842 | nM | 138 | 5.6 |
| 81 | 2.00E+005 | 2.60E−002 | 126 | nM | 283 | 6.2 |
| 82 | 1.50E+004 | 3.00E−003 | 204 | nM | 131 | 11 |
| 83 | 2.60E+005 | 1.80E−002 | 70 | nM | 248 | 4 |
| 84 | 1.50E+004 | 1.90E−003 | 125 | nM | 21 | 1.9 |
| 85 | 1.20E+005 | 6.80E−002 | 554 | nM | 173 | 4.4 |
| 86 | 7.20E+005 | 2.90E−002 | 41 | nM | 322 | 8.8 |
| 87 | 2.70E+004 | 1.80E−002 | 642 | nM | 126 | 6.4 |
| 88 | 2.40E+005 | 1.30E−001 | 543 | nM | 183 | 3.2 |
| 89 | 2.40E+005 | 2.40E−002 | 102 | nM | 233 | 5.3 |
| 90 | 1.70E+005 | 1.70E−001 | 970 | nM | 223 | 4.2 |
| 91 | 1.60E+005 | 4.10E−002 | 256 | nM | 263 | 4.7 |
| 92 | 2.00E+005 | 1.30E−001 | 662 | nM | 229 | 6.1 |
| 93 | 1.50E+005 | 1.10E−001 | 728 | nM | 218 | 6.1 |
| 94 | 1.30E+005 | 6.20E−002 | 483 | nM | 229 | 9.3 |
| 95 | 1.90E+004 | 8.60E−003 | 446 | nM | 53 | 3.5 |
| 96 | 2.90E+005 | 9.70E−002 | 337 | nM | 252 | 7.4 |
| 97 | 2.00E+005 | 6.30E−003 | 31 | nM | 297 | 12 |
| 98 | 2.70E+005 | 2.50E−002 | 95 | nM | 227 | 5.8 |
| 99 | 4.60E+005 | 8.90E−002 | 192 | nM | 231 | 6.9 |
| 100 | 2.20E+005 | 7.40E−002 | 342 | nM | 213 | 4 |
| 101 | 1.90E+005 | 1.10E−001 | 590 | nM | 227 | 7 |
| 102 | 2.40E+005 | 6.50E−002 | 270 | nM | 286 | 5.5 |
| 103 | 4.50E+004 | 6.30E−003 | 140 | nM | 96 | 7.5 |
| 104 | 5.10E+005 | 6.10E−002 | 121 | nM | 276 | 5.7 |
| 105 | 3.70E+005 | 9.50E−002 | 256 | nM | 277 | 6.5 |
| 106 | 2.40E+005 | 1.50E−001 | 616 | nM | 247 | 7.2 |
| 107 | 1.30E+005 | 6.20E−002 | 465 | nM | 212 | 9.1 |
| 108 | 2.00E+005 | 5.60E−003 | 28 | nM | 313 | 9.3 |
| 109 | 6.00E+005 | 6.10E−003 | 10 | nM | 308 | 7.3 |
| ST2 Antibody | Mouse ST2 | | | | | |
| # | ka (M−1 s−1) | kd (s−1) | KD (M) | | Rmax | Res sd |
| 1 | N/A | N/A | N/A | | N/A | N/A |
| 2 | N/A | N/A | N/A | | N/A | N/A |
| 3 | N/A | N/A | N/A | | N/A | N/A |
| 4 | N/A | N/A | N/A | | N/A | N/A |
| 5 | N/A | N/A | N/A | | N/A | N/A |
| 6 | 3.500E+004 | 1.800E−001 | 5.0 | uM | 2.430E+002 | 1.700E+000 |
| 7 | 6.90E+004 | 1.30E−001 | 1.9 | uM | 168 | 2.5 |
| 8 | N/A | N/A | N/A | | N/A | N/A |
| 9 | 9.10E+004 | 3.20E−002 | 351 | nM | 273 | 3.2 |
| 10 | N/A | N/A | N/A | | N/A | N/A |
| 11 | N/A | N/A | N/A | | N/A | N/A |
| 12 | 3.60E+004 | 5.30E−002 | 1.5 | uM | 244 | 2.5 |
| 13 | N/A | N/A | N/A | | N/A | N/A |
| 14 | N/A | N/A | N/A | | N/A | N/A |
| 15 | N/A | N/A | N/A | | N/A | N/A |
| 16 | 3.20E+005 | 1.00E−001 | 315 | nM | 260 | 5.5 |
| 17 | N/A | N/A | N/A | | N/A | N/A |
| 18 | N/A | N/A | N/A | | N/A | N/A |
| 19 | 3.70E+005 | 1.10E−001 | 311 | nM | 244 | 4.9 |
| 20 | 7.70E+004 | 1.70E−001 | 2.2 | uM | 165 | 2.3 |
| 21 | 8.50E+003 | 8.50E−003 | 999 | nM | 38 | 1.1 |
| 22 | 1.20E+005 | 1.40E−001 | 1.1 | uM | 165 | 2.8 |
| 23 | N/A | N/A | N/A | | N/A | N/A |
| 24 | N/A | N/A | N/A | | N/A | N/A |
| 25 | 2.00E+005 | 6.40E−002 | 322 | nM | 274 | 5 |
| 26 | N/A | N/A | N/A | | N/A | N/A |
| 27 | N/A | N/A | N/A | | N/A | N/A |
| 28 | N/A | N/A | N/A | | N/A | N/A |
| 29 | 5.90E+003 | 4.50E−003 | 751 | nM | 50 | 1.2 |
| 30 | 4.80E+004 | 1.30E−001 | 2.7 | uM | 188 | 1.1 |
| 31 | N/A | N/A | N/A | | N/A | N/A |
| 32 | N/A | N/A | N/A | | N/A | N/A |
| 33 | N/A | N/A | N/A | | N/A | N/A |
| 34 | 1.50E+004 | 1.90E−003 | 126 | nM | 125 | 8.4 |
| 35 | 1.70E+005 | 1.80E−002 | 107 | nM | 291 | 5.4 |
| 36 | N/A | N/A | N/A | | N/A | N/A |
| 37 | 4.20E+004 | 6.10E−002 | 1.4 | uM | 56 | 2.5 |
| 38 | 1.70E+005 | 9.00E−002 | 529 | nM | 207 | 2.7 |
| 39 | 4.20E+004 | 1.40E−001 | 3.4 | uM | 172 | 2.2 |
| 40 | N/A | N/A | N/A | | N/A | N/A |
| 41 | 1.40E+005 | 1.10E−001 | 845 | nM | 254 | 3.6 |
| 42 | N/A | N/A | N/A | | N/A | N/A |
| 43 | N/A | N/A | N/A | | N/A | N/A |
| 44 | 1.10E+005 | 1.10E−001 | 999 | nM | 243 | 3.6 |
| 45 | 9.50E+004 | 1.30E−001 | 1.4 | uM | 257 | 2.5 |
| 46 | 3.30E+004 | 1.60E−001 | 5.0 | uM | 228 | 1.1 |
| 47 | N/A | N/A | N/A | | N/A | N/A |
| 48 | N/A | N/A | N/A | | N/A | N/A |
| 49 | 2.70E+005 | 1.20E−001 | 436 | nM | 246 | 4.5 |
| 50 | N/A | N/A | N/A | | N/A | N/A |
| 51 | 8.30E+004 | 1.70E−001 | 2.1 | uM | 245 | 1.8 |
| 52 | 5.90E+004 | 1.60E−001 | 2.7 | uM | 200 | 1.9 |
| 53 | N/A | N/A | N/A | | N/A | N/A |
| 54 | 5.00E+004 | 1.30E−001 | 2.7 | uM | 89 | 1.5 |
| 55 | N/A | N/A | N/A | | N/A | N/A |
| 56 | N/A | N/A | N/A | | N/A | N/A |
| 57 | 5.10E+004 | 1.60E−001 | 3.1 | uM | 125 | 1.9 |
| 58 | N/A | N/A | N/A | | N/A | N/A |
| 59 | N/A | N/A | N/A | | N/A | N/A |
| 60 | N/A | N/A | N/A | | N/A | N/A |
| 61 | N/A | N/A | N/A | | N/A | N/A |
| 62 | N/A | N/A | N/A | | N/A | N/A |
| 63 | 6.20E+004 | 1.50E−001 | 2.4 | uM | 142 | 1.5 |
| 64 | N/A | N/A | N/A | | N/A | N/A |
| 65 | N/A | N/A | N/A | | N/A | N/A |
| 66 | N/A | N/A | N/A | | N/A | N/A |
| 67 | N/A | N/A | N/A | | N/A | N/A |
| 68 | N/A | N/A | N/A | | N/A | N/A |
| 69 | N/A | N/A | N/A | | N/A | N/A |
| 70 | 3.30E+004 | 1.30E−001 | 3.9 | uM | 117 | 1.6 |
| 71 | N/A | N/A | N/A | | N/A | N/A |
| 72 | N/A | N/A | N/A | | N/A | N/A |
| 73 | N/A | N/A | N/A | | N/A | N/A |
| 74 | 9.60E+003 | 1.20E−001 | 12 | uM | 257 | 1.2 |
| 75 | N/A | N/A | N/A | | N/A | N/A |
| 76 | 1.80E+005 | 1.40E−001 | 778 | nM | 214 | 3.3 |
| 77 | N/A | N/A | N/A | | N/A | N/A |
| 78 | 1.80E+005 | 1.30E−001 | 744 | nM | 178 | 2.6 |
| 79 | N/A | N/A | N/A | | N/A | N/A |
| 80 | N/A | N/A | N/A | | N/A | N/A |
| 81 | N/A | N/A | N/A | | N/A | N/A |
| 82 | N/A | N/A | N/A | | N/A | N/A |
| 83 | 1.40E+005 | 6.30E−002 | 438 | nM | 216 | 3.2 |
| 84 | N/A | N/A | N/A | | N/A | N/A |
| 85 | N/A | N/A | N/A | | N/A | N/A |
| 86 | 1.90E+005 | 9.80E−002 | 507 | nM | 262 | 5.4 |
| 87 | N/A | N/A | N/A | | N/A | N/A |
| 88 | 6.10E+004 | 9.30E−002 | 1.5 | uM | 105 | 3 |
| 89 | 1.00E+005 | 1.00E−001 | 989 | nM | 188 | 2.6 |
| 90 | N/A | N/A | N/A | | N/A | N/A |
| 91 | N/A | N/A | N/A | | N/A | N/A |
| 92 | N/A | N/A | N/A | | N/A | N/A |
| 93 | N/A | N/A | N/A | | N/A | N/A |
| 94 | 3.70E+004 | 8.60E−002 | 2.3 | uM | 167 | 2.4 |
| 95 | N/A | N/A | N/A | | N/A | N/A |
| 96 | N/A | N/A | N/A | | N/A | N/A |
| 97 | N/A | N/A | N/A | | N/A | N/A |
| 98 | 1.20E+005 | 1.20E−001 | 975 | nM | 181 | 2.6 |
| 99 | 2.30E+005 | 9.80E−002 | 419 | nM | 202 | 4.4 |
| 100 | N/A | N/A | N/A | | N/A | N/A |
| 101 | N/A | N/A | N/A | | N/A | N/A |
| 102 | N/A | N/A | N/A | | N/A | N/A |
| 103 | 2.70E+004 | 5.80E−003 | 211 | nM | 78 | 3.3 |
| 104 | 1.40E+005 | 9.60E−002 | 684 | nM | 222 | 3.4 |
| 105 | 4.10E+005 | 7.00E−002 | 172 | nM | 273 | 5.5 |
| 106 | N/A | N/A | N/A | | N/A | N/A |
| 107 | N/A | N/A | N/A | | N/A | N/A |
| 108 | N/A | N/A | N/A | | N/A | N/A |
| 109 | 1.10E+006 | 1.30E−003 | 1.1 | nM | 140 | 16 |

### Example 5. Evaluation of Bispecific Molecules in Activation of ST2+ Tregs in Mouse Spleen (Prophetic)

ST2+ Treg are found in several human tissues at high levels, but are found in blood at a very low frequency, <0.01%. Due to the difficulty of obtaining tissues from human donors, the effect of bispecific Fc fusion proteins containing an IL-2 variant and an ST2 antibody will be assessed on ST2+ Treg from mouse spleen, which was found to have higher levels of ST2+ Tregs (5-10% of Tregs), more than were found in blood (0.1-1.0% of Tregs). Spleens will be isolated from C57Bl/6J mice, and single cell suspensions stimulated with a range of concentrations of either a monovalent IL-2 variant Fc fusion, a monovalent ST2 antibody Fc fusion or a bispecific IL-2 variant/ST2 antibody Fc fusion. Treg activation by IL-2 will be measured by determining the level of intracellular phosphorylated STAT5 (pSTAT5) by flow cytometry. It is expected that the bispecific protein will enhance pSTAT5 induction in ST2+ Treg, above levels seen with either the IL-2 variant Fc fusion or ST2 antibody Fc fusion proteins alone, but not in ST2− Treg demonstrating that the bispecific molecule preferentially activates ST2+ Treg.

### Example 6. Activity of IL2/ST2 Antibody Bispecific Molecule in Normal Mice (Prophetic)

To determine their activity on Treg populations in normal mice, BALB/c mice will be injected intravenously with a single dose of 0.001, 0.01, or 0.1 mg/kg of either an IL-2 variant/Fc fusion proteins, an ST2 antibody/Fc fusion proteins, or a bispecific IL2-ST2 antibody fusion protein (e.g. FIG. 3). Spleens and livers will be harvested 2, 4, 6 or 8 days after treatment, and numbers and percentages (as a fraction of CD4 cells) of ST2+ Treg will be determined. In addition, the proliferative index of the ST2+ and ST2− Treg subsets will be determined by intracellular staining of cells with antibody to Ki67.

If a bispecific IL2-ST2 antibody Fc fusion has greater selectivity for ST2+ Tregs than the IL2 variant Fc fusion and ST2 antibody Fc fusion, greater expansion of ST2+ Tregs than ST2-Tregs will be observed upon treatment with the bispecific protein compared to the IL2 variant Fc fusion and the ST2 antibody Fc fusion. An increase in the proliferative index, as reflected by the percent Ki67+ cells, will also result from treatment with the bispecific proteins compared to the IL2 variant Fc fusion and the ST2 antibody Fc fusion. The effects of the proteins on Tregs can be correlated with the pharmacokinetics of the administered proteins. Blood samples taken after administration will be evaluated by a quantitative immunoassay to determine the pharmacokinetics of administered molecules.

### Example 7. Activity in Models of Muscle Inflammation (Prophetic)

The role of ST2+ Tregs has been established in animal models of muscle inflammation. One of those animal models is acute muscle injury (Burzyn et al., 2013, Cell 155(6): 1282-1295) in wild type mice, and a second model is the mdx mouse muscular dystrophy model, a model of chronic muscle inflammation caused by genetic deficiency in dystrophin (mdx mice; Villalta et al., 2014, Sci Transl Med 5(258): 258ra142).

Acute muscle injury will be initiated in mice by the injection of cardiotoxin into hind limb muscles of C57Bl/6J mice, as described by Burzyn et al. (cited above). Treatment with 0.1 mg/kg of IL-2-ST2 antibody bispecific molecules (e.g. FIGS. 3A and 3B) will be initiated on the day of injury, and again on day 7. Mice will be sacrificed on day 1, 4, 7 and 14, and the number of Tregs, Teff and other infiltrating immune cells in the muscle will be determined by flow cytometry. Amphiregulin (AREG) production by Treg is a crucial mediator of muscle repair (Burzyn et al., cited above), and the frequency of AREG+ Treg, and the proliferative index (Ki67+ Treg) will be determined by intracellular flow cytometry. Measures of muscle injury and repair, such as creatine kinase levels in the serum and muscle fiber morphology will also be assessed.

For the mouse mdx muscular dystrophy model, treatment of mdx mice will be initiated at 2 weeks of age. Mice will be treated weekly with 0.1 mg/kg of test proteins and sacrificed at 6 weeks of age. The number and frequency of proliferating Treg (Ki67+) and AREG+ Treg will be measured in muscles of treated mice compared to age-matched untreated controls.

Successful activation of ST2+ Tregs will result in a numerical increase in Treg, a higher proportion of Ki67+ Treg, or a higher proportion of AREG+ Treg in muscle. Additionally, the mice may exhibit a reduction in Teff cells, decreased serum creatine kinase, and improved muscle morphology.

### Example 8. Activity in Models of Inflammatory Bowel Disease (Prophetic)

A role for ST2+ Treg has been established in a mouse model of inflammatory bowel disease (Schiering et al., 2014, Nature 513(7519):564-568). The effect of test proteins on ST2+ Treg in colonic tissue will be tested in an acute model of inflammatory bowel disease. C57Bl/6J mice will be fed 3% dextran sodium sulfate (DSS) in the drinking water for 7 days. Mice will be treated IV, IP, or SC with 0.1 or 0.4 mg/kg of IL-2/ST2 antibody bispecific molecules (e.g. FIGS. 3A and 3B) on day 1 and day 4 of DSS treatment, with DSS treatment starting on day 1. After the 7 day DSS treatment, mice will be sacrificed, and spleens, colons and mesenteric lymph nodes (MLNs) harvested. Colon sections will analyzed by histology for disease severity and colitis scores. ST2+ Treg populations will be measured in spleens, colons and MLNs.

Successful treatment could result in reduced weight loss, improved disease scores or histology in treated mice compared to controls. Disease improvement might be accompanied by a specific increase in Ki67+ proliferating ST2+ Treg in the colons and MLNs of treated mice.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

We claim:

1. A recombinant antibody or an antigen-binding fragment thereof that specifically binds ST2, comprising:
(i) a light chain variable region having at least 85% sequence identity to an amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457; and
(ii) a heavy chain variable region having at least 90% sequence identity to an amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239.

2. The antibody or antigen-binding fragment of claim 1, comprising:
a light chain variable region comprising amino acids 21-132 of SEQ ID NO: 241 and a heavy chain variable region comprising amino acids 25-147 of SEQ ID NO: 23;
a light chain variable region comprising amino acids 21-132 of SEQ ID NO: 243 and a heavy chain variable region comprising amino acids 25-147 of SEQ ID NO: 25;
a light chain variable region comprising amino acids 21-128 of SEQ ID NO: 245 and a heavy chain variable region comprising amino acids 25-145 of SEQ ID NO: 27;
a light chain variable region comprising amino acids 21-127 of SEQ ID NO: 247 and a heavy chain variable region comprising amino acids 25-148 of SEQ ID NO: 29;
a light chain variable region comprising amino acids 21-127 of SEQ ID NO: 249 and a heavy chain variable region comprising amino acids 25-141 of SEQ ID NO: 31;
a light chain variable region comprising amino acids 21-127 of SEQ ID NO: 251 and a heavy chain variable region comprising amino acids 25-143 of SEQ ID NO: 33;
a light chain variable region comprising amino acids 21-131 of SEQ ID NO: 253 and a heavy chain variable region comprising amino acids 25-150 of SEQ ID NO: 35;
a light chain variable region comprising amino acids 21-132 of SEQ ID NO: 255 and a heavy chain variable region comprising amino acids 25-148 of SEQ ID NO: 37;
a light chain variable region comprising amino acids 21-127 of SEQ ID NO: 257 and a heavy chain variable region comprising amino acids 25-146 of SEQ ID NO: 39;
a light chain variable region comprising amino acids 21-132 of SEQ ID NO: 259 and a heavy chain variable region comprising amino acids 25-145 of SEQ ID NO: 41;
a light chain variable region comprising amino acids 21-127 of SEQ ID NO: 261 and a heavy chain variable region comprising amino acids 25-143 of SEQ ID NO: 43;
a light chain variable region comprising amino acids 21-127 of SEQ ID NO: 263 and a heavy chain variable region comprising amino acids 25-151 of SEQ ID NO: 45;
a light chain variable region comprising amino acids 21-132 of SEQ ID NO: 265 and a heavy chain variable region comprising amino acids 25-141 of SEQ ID NO: 47;
a light chain variable region comprising amino acids 21-132 of SEQ ID NO: 267 and a heavy chain variable region comprising amino acids 25-140 of SEQ ID NO: 49;
a light chain variable region comprising amino acids 21-127 of SEQ ID NO: 269 and a heavy chain variable region comprising amino acids 25-145 of SEQ ID NO: 51;
a light chain variable region comprising amino acids 1-127 of SEQ ID NO: 271 and a heavy chain variable region comprising amino acids 25-152 of SEQ ID NO: 53;
a light chain variable region comprising amino acids 21-127 of SEQ ID NO: 273 and a heavy chain variable region comprising amino acids 25-142 of SEQ ID NO: 55;
a light chain variable region comprising amino acids 21-127 of SEQ ID NO: 275 and a heavy chain variable region comprising amino acids 25-147 of SEQ ID NO: 57;
a light chain variable region comprising amino acids 21-127 of SEQ ID NO: 277 and a heavy chain variable region comprising amino acids 25-141 of SEQ ID NO: 59;
a light chain variable region comprising amino acids 21-127 of SEQ ID NO: 279 and a heavy chain variable region comprising amino acids 25-148 of SEQ ID NO: 61;
a light chain variable region comprising amino acids 21-127 of SEQ ID NO: 281 and a heavy chain variable region comprising amino acids 25-142 of SEQ ID NO: 63;
a light chain variable region comprising amino acids 21-127 of SEQ ID NO: 283 and a heavy chain variable region comprising amino acids 25-145 of SEQ ID NO: 65;
a light chain variable region comprising amino acids 21-127 of SEQ ID NO: 285 and a heavy chain variable region comprising amino acids 25-140 of SEQ ID NO: 67;
a light chain variable region comprising amino acids 21-132 of SEQ ID NO: 287 and a heavy chain variable region comprising amino acids 25-145 of SEQ ID NO: 69;
a light chain variable region comprising amino acids 21-127 of SEQ ID NO: 289 and a heavy chain variable region comprising amino acids 25-140 of SEQ ID NO: 71;
a light chain variable region comprising amino acids 21-133 of SEQ ID NO: 291 and a heavy chain variable region comprising amino acids 25-140 of SEQ ID NO: 73;
a light chain variable region comprising amino acids 21-127 of SEQ ID NO: 293 and a heavy chain variable region comprising amino acids 25-145 of SEQ ID NO: 75;
a light chain variable region comprising amino acids 21-132 of SEQ ID NO: 295 and a heavy chain variable region comprising amino acids 25-143 of SEQ ID NO: 77;
a light chain variable region comprising amino acids 21-127 of SEQ ID NO: 297 and a heavy chain variable region comprising amino acids 25-143 of SEQ ID NO: 79;
a light chain variable region comprising amino acids 21-132 of SEQ ID NO: 299 and a heavy chain variable region comprising amino acids 25-151 of SEQ ID NO: 81;
a light chain variable region comprising amino acids 21-127 of SEQ ID NO: 301 and a heavy chain variable region comprising amino acids 25-142 of SEQ ID NO: 83;
a light chain variable region comprising amino acids 21-127 of SEQ ID NO: 303 and a heavy chain variable region comprising amino acids 25-144 of SEQ ID NO: 85;
a light chain variable region comprising amino acids 21-132 of SEQ ID NO: 305 and a heavy chain variable region comprising amino acids 25-148 of SEQ ID NO: 87;
a light chain variable region comprising amino acids 21-127 of SEQ ID NO: 307 and a heavy chain variable region comprising amino acids 25-144 of SEQ ID NO: 89;
a light chain variable region comprising amino acids 21-127 of SEQ ID NO: 309 and a heavy chain variable region comprising amino acids 25-140 of SEQ ID NO: 91;
a light chain variable region comprising amino acids 21-127 of SEQ ID NO: 311 and a heavy chain variable region comprising amino acids 25-143 of SEQ ID NO: 93;
a light chain variable region comprising amino acids 21-127 of SEQ ID NO: 313 and a heavy chain variable region comprising amino acids 25-150 of SEQ ID NO: 95;
a light chain variable region comprising amino acids 21-128 of SEQ ID NO: 315 and a heavy chain variable region comprising amino acids 25-147 of SEQ ID NO: 97;
a light chain variable region comprising amino acids 21-132 of SEQ ID NO: 317 and a heavy chain variable region comprising amino acids 25-141 of SEQ ID NO: 99;
a light chain variable region comprising amino acids 21-127 of SEQ ID NO: 319 and a heavy chain variable region comprising amino acids 25-153 of SEQ ID NO: 101;
a light chain variable region comprising amino acids 21-133 of SEQ ID NO: 321 and a heavy chain variable region comprising amino acids 25-152 of SEQ ID NO: 103;
a light chain variable region comprising amino acids 21-127 of SEQ ID NO: 323 and a heavy chain variable region comprising amino acids 25-145 of SEQ ID NO: 105;
a light chain variable region comprising amino acids 21-127 of SEQ ID NO: 325 and a heavy chain variable region comprising amino acids 25-144 of SEQ ID NO: 107;
a light chain variable region comprising amino acids 21-127 of SEQ ID NO: 327 and a heavy chain variable region comprising amino acids 25-146 of SEQ ID NO: 109;
a light chain variable region comprising amino acids 21-127 of SEQ ID NO: 329 and a heavy chain variable region comprising amino acids 25-140 of SEQ ID NO: 111;
a light chain variable region comprising amino acids 21-127 of SEQ ID NO: 331 and a heavy chain variable region comprising amino acids 25-143 of SEQ ID NO: 113;
a light chain variable region comprising amino acids 21-127 of SEQ ID NO: 333 and a heavy chain variable region comprising amino acids 25-144 of SEQ ID NO: 115;
a light chain variable region comprising amino acids 21-127 of SEQ ID NO: 335 and a heavy chain variable region comprising amino acids 25-141 of SEQ ID NO: 117;
a light chain variable region comprising amino acids 21-127 of SEQ ID NO: 337 and a heavy chain variable region comprising amino acids 25-149 of SEQ ID NO: 119;
a light chain variable region comprising amino acids 21-127 of SEQ ID NO: 339 and a heavy chain variable region comprising amino acids 25-145 of SEQ ID NO: 121;
a light chain variable region comprising amino acids 21-128 of SEQ ID NO: 341 and a heavy chain variable region comprising amino acids 25-149 of SEQ ID NO: 123;
a light chain variable region comprising amino acids 21-131 of SEQ ID NO: 343 and a heavy chain variable region comprising amino acids 25-149 of SEQ ID NO: 125;
a light chain variable region comprising amino acids 21-127 of SEQ ID NO: 345 and a heavy chain variable region comprising amino acids 25-147 of SEQ ID NO: 127;
a light chain variable region comprising amino acids 21-131 of SEQ ID NO: 347 and a heavy chain variable region comprising amino acids 25-147 of SEQ ID NO: 129;
a light chain variable region comprising amino acids 21-132 of SEQ ID NO: 349 and a heavy chain variable region comprising amino acids 25-145 of SEQ ID NO: 131;
a light chain variable region comprising amino acids 21-127 of SEQ ID NO: 351 and a heavy chain variable region comprising amino acids 25-146 of SEQ ID NO: 133;
a light chain variable region comprising amino acids 21-127 of SEQ ID NO: 353 and a heavy chain variable region comprising amino acids 25-152 of SEQ ID NO: 135;
a light chain variable region comprising amino acids 21-127 of SEQ ID NO: 355 and a heavy chain variable region comprising amino acids 25-146 of SEQ ID NO: 137;
a light chain variable region comprising amino acids 21-127 of SEQ ID NO: 357 and a heavy chain variable region comprising amino acids 25-149 of SEQ ID NO: 139;
a light chain variable region comprising amino acids 21-127 of SEQ ID NO: 359 and a heavy chain variable region comprising amino acids 25-149 of SEQ ID NO: 141;
a light chain variable region comprising amino acids 21-132 of SEQ ID NO: 361 and a heavy chain variable region comprising amino acids 25-145 of SEQ ID NO: 143;
a light chain variable region comprising amino acids 21-133 of SEQ ID NO: 363 and a heavy chain variable region comprising amino acids 25-142 of SEQ ID NO: 145;
a light chain variable region comprising amino acids 21-132 of SEQ ID NO: 365 and a heavy chain variable region comprising amino acids 25-147 of SEQ ID NO: 147;
a light chain variable region comprising amino acids 21-126 of SEQ ID NO: 367 and a heavy chain variable region comprising amino acids 25-141 of SEQ ID NO: 149;
a light chain variable region comprising amino acids 21-127 of SEQ ID NO: 369 and a heavy chain variable region comprising amino acids 25-140 of SEQ ID NO: 151;
a light chain variable region comprising amino acids 21-132 of SEQ ID NO: 371 and a heavy chain variable region comprising amino acids 25-145 of SEQ ID NO: 153;
a light chain variable region comprising amino acids 21-127 of SEQ ID NO: 373 and a heavy chain variable region comprising amino acids 25-153 of SEQ ID NO: 155;
a light chain variable region comprising amino acids 21-132 of SEQ ID NO: 375 and a heavy chain variable region comprising amino acids 25-146 of SEQ ID NO: 157;
a light chain variable region comprising amino acids 21-132 of SEQ ID NO: 377 and a heavy chain variable region comprising amino acids 25-149 of SEQ ID NO: 159;
a light chain variable region comprising amino acids 21-128 of SEQ ID NO: 379 and a heavy chain variable region comprising amino acids 25-141 of SEQ ID NO: 161;
a light chain variable region comprising amino acids 21-127 of SEQ ID NO: 381 and a heavy chain variable region comprising amino acids 25-156 of SEQ ID NO: 163;
a light chain variable region comprising amino acids 21-127 of SEQ ID NO: 383 and a heavy chain variable region comprising amino acids 25-141 of SEQ ID NO: 165;
a light chain variable region comprising amino acids 21-127 of SEQ ID NO: 385 and a heavy chain variable region comprising amino acids 25-140 of SEQ ID NO: 167;
a light chain variable region comprising amino acids 21-127 of SEQ ID NO: 387 and a heavy chain variable region comprising amino acids 25-140 of SEQ ID NO: 169;
a light chain variable region comprising amino acids 21-127 of SEQ ID NO: 389 and a heavy chain variable region comprising amino acids 25-141 of SEQ ID NO: 171;
a light chain variable region comprising amino acids 21-127 of SEQ ID NO: 391 and a heavy chain variable region comprising amino acids 25-140 of SEQ ID NO: 173;
a light chain variable region comprising amino acids 21-133 of SEQ ID NO: 393 and a heavy chain variable region comprising amino acids 25-144 of SEQ ID NO: 175;
a light chain variable region comprising amino acids 21-127 of SEQ ID NO: 395 and a heavy chain variable region comprising amino acids 25-142 of SEQ ID NO: 177;
a light chain variable region comprising amino acids 21-127 of SEQ ID NO: 397 and a heavy chain variable region comprising amino acids 25-145 of SEQ ID NO: 179;
a light chain variable region comprising amino acids 21-132 of SEQ ID NO: 399 and a heavy chain variable region comprising amino acids 25-145 of SEQ ID NO: 181;
a light chain variable region comprising amino acids 21-127 of SEQ ID NO: 401 and a heavy chain variable region comprising amino acids 25-143 of SEQ ID NO: 183;
a light chain variable region comprising amino acids 21-127 of SEQ ID NO: 403 and a heavy chain variable region comprising amino acids 25-147 of SEQ ID NO: 185;
a light chain variable region comprising amino acids 21-127 of SEQ ID NO: 405 and a heavy chain variable region comprising amino acids 25-143 of SEQ ID NO: 187;
a light chain variable region comprising amino acids 21-132 of SEQ ID NO: 407 and a heavy chain variable region comprising amino acids 25-145 of SEQ ID NO: 189;
a light chain variable region comprising amino acids 21-127 of SEQ ID NO: 409 and a heavy chain variable region comprising amino acids 25-144 of SEQ ID NO: 191;
a light chain variable region comprising amino acids 21-127 of SEQ ID NO: 411 and a heavy chain variable region comprising amino acids 25-143 of SEQ ID NO: 193;
a light chain variable region comprising amino acids 21-127 of SEQ ID NO: 413 and a heavy chain variable region comprising amino acids 25-146 of SEQ ID NO: 195;
a light chain variable region comprising amino acids 21-127 of SEQ ID NO: 415 and a heavy chain variable region comprising amino acids 25-141 of SEQ ID NO: 197;
a light chain variable region comprising amino acids 21-127 of SEQ ID NO: 417 and a heavy chain variable region comprising amino acids 25-146 of SEQ ID NO: 199;
a light chain variable region comprising amino acids 21-132 of SEQ ID NO: 419 and a heavy chain variable region comprising amino acids 25-142 of SEQ ID NO: 201;
a light chain variable region comprising amino acids 21-127 of SEQ ID NO: 421 and a heavy chain variable region comprising amino acids 25-139 of SEQ ID NO: 203;
a light chain variable region comprising amino acids 21-133 of SEQ ID NO: 423 and a heavy chain variable region comprising amino acids 25-144 of SEQ ID NO: 205;
a light chain variable region comprising amino acids 21-132 of SEQ ID NO: 425 and a heavy chain variable region comprising amino acids 25-146 of SEQ ID NO: 207;
a light chain variable region comprising amino acids 21-128 of SEQ ID NO: 427 and a heavy chain variable region comprising amino acids 25-142 of SEQ ID NO: 209;
a light chain variable region comprising amino acids 21-132 of SEQ ID NO: 429 and a heavy chain variable region comprising amino acids 25-151 of SEQ ID NO: 211;
a light chain variable region comprising amino acids 21-132 of SEQ ID NO: 431 and a heavy chain variable region comprising amino acids 25-141 of SEQ ID NO: 213;
a light chain variable region comprising amino acids 21-127 of SEQ ID NO: 433 and a heavy chain variable region comprising amino acids 25-140 of SEQ ID NO: 215;
a light chain variable region comprising amino acids 21-127 of SEQ ID NO: 435 and a heavy chain variable region comprising amino acids 25-146 of SEQ ID NO: 217;
a light chain variable region comprising amino acids 21-127 of SEQ ID NO: 437 and a heavy chain variable region comprising amino acids 25-142 of SEQ ID NO: 219;
a light chain variable region comprising amino acids 21-127 of SEQ ID NO: 439 and a heavy chain variable region comprising amino acids 25-143 of SEQ ID NO: 221;
a light chain variable region comprising amino acids 21-126 of SEQ ID NO: 441 and a heavy chain variable region comprising amino acids 25-150 of SEQ ID NO: 223;
a light chain variable region comprising amino acids 21-132 of SEQ ID NO: 443 and a heavy chain variable region comprising amino acids 25-144 of SEQ ID NO: 225;
a light chain variable region comprising amino acids 21-127 of SEQ ID NO: 445 and a heavy chain variable region comprising amino acids 25-145 of SEQ ID NO: 227;
a light chain variable region comprising amino acids 21-127 of SEQ ID NO: 447 and a heavy chain variable region comprising amino acids 25-149 of SEQ ID NO: 229;
a light chain variable region comprising amino acids 21-127 of SEQ ID NO: 449 and a heavy chain variable region comprising amino acids 251-147 of SEQ ID NO: 231;
a light chain variable region comprising amino acids 21-128 of SEQ ID NO: 451 and a heavy chain variable region comprising amino acids 25-140 of SEQ ID NO: 233;
a light chain variable region comprising amino acids 21-127 of SEQ ID NO: 453 and a heavy chain variable region comprising amino acids 25-141 of SEQ ID NO: 235;
a light chain variable region comprising amino acids 21-127 of SEQ ID NO: 455 and a heavy chain variable region comprising amino acids 25-140 of SEQ ID NO: 237; or
a light chain variable region comprising amino acids 21-133 of SEQ ID NO: 457 and a heavy chain variable region comprising amino acids 25-150 of SEQ ID NO: 239.

3. The antibody or antigen-binding fragment of claim 1, comprising a heavy chain fragment selected from the group consisting of:
amino acids 25-251 of SEQ ID NO: 23, amino acids 25-250 of SEQ ID NO: 25, amino acids 25-249 of SEQ ID NO: 27, amino acids 25-252 of SEQ ID NO: 29, amino acids 25-245 of SEQ ID NO: 31, amino acids 25-247 of SEQ ID NO: 33, amino acids 25-254 of SEQ ID NO: 35, amino acids 25-252 of SEQ ID NO: 37, amino acids 25-250 of SEQ ID NO: 39, amino acids 25-249 of SEQ ID NO: 41, amino acids 25-248 of SEQ ID NO: 43, amino acids 25-255 of SEQ ID NO: 45, amino acids 25-245 of SEQ ID NO: 47, amino acids 25-244 of SEQ ID NO: 49, amino acids 25-249 of SEQ ID NO: 51, amino acids 25-256 of SEQ ID NO: 53, amino acids 25-246 of SEQ ID NO: 55, amino acids 25-251 of SEQ ID NO: 57, amino acids 25-245 of SEQ ID NO: 59, amino acids 25-252 of SEQ ID NO: 61, amino acids 25-246 of SEQ ID NO: 63, amino acids 25-249 of SEQ ID NO: 65, amino acids 25-244 of SEQ ID NO: 67, amino acids 25-249 of SEQ ID NO: 69, amino acids 25-244 of SEQ ID NO: 71, amino acids 25-244 of SEQ ID NO: 73, amino acids 25-249 of SEQ ID NO: 75, amino acids 25-247 of SEQ ID NO: 77, amino acids 25-247 of SEQ ID NO: 79, amino acids 25-255 of SEQ ID NO: 81, amino acids 25-246 of SEQ ID NO: 83, amino acids 25-248 of SEQ ID NO: 85, amino acids 25-252 of SEQ ID NO: 87, amino acids 25-248 of SEQ ID NO: 89, amino acids 25-244 of SEQ ID NO: 91, amino acids 25-247 of SEQ ID NO: 93, amino acids 25-254 of SEQ ID NO: 95, amino acids 25-251 of SEQ ID NO: 97, amino acids 25-245 of SEQ ID NO: 99, amino acids 25-257 of SEQ ID NO: 101, amino acids 25-256 of SEQ ID NO: 103, amino acids 25-249 of SEQ ID NO: 105, amino acids 25-248 of SEQ ID NO: 107, amino acids 25-250 of SEQ ID NO: 109, amino acids 25-244 of SEQ ID NO: 111, amino acids 25-247 of SEQ ID NO: 113, amino acids 25-248 of SEQ ID NO: 115, amino acids 25-245 of SEQ ID NO: 117, amino acids 25-253 of SEQ ID NO: 119, amino acids 25-249 of SEQ ID NO: 121, amino acids 25-253 of SEQ ID NO: 123, amino acids 25-253 of SEQ ID NO: 125, amino acids 25-251 of SEQ ID NO: 127, amino acids 25-245 of SEQ ID NO: 129, amino acids 25-249 of SEQ ID NO: 131, amino acids 25-250 of SEQ ID NO: 133, amino acids 25-256 of SEQ ID NO: 135, amino acids 25-250 of SEQ ID NO: 137, amino acids 25-247 of SEQ ID NO: 139, amino acids 25-253 of SEQ ID NO: 141, amino acids 25-249 of SEQ ID NO: 143, amino acids 25-246 of SEQ ID NO: 145, amino acids 25-251 of SEQ ID NO: 147, amino acids 25-245 of SEQ ID NO: 149, amino acids 25-244 of SEQ ID NO: 151, amino acids 25-249 of SEQ ID NO: 153, amino acids 25-257 of SEQ ID NO: 155, amino acids 25-250 of SEQ ID NO: 157, amino acids 25-253 of SEQ ID NO: 159, amino acids 25-245 of SEQ ID NO: 161, amino acids 25-260 of SEQ ID NO: 163, amino acids 25-245 of SEQ ID NO: 165, amino acids 25-244 of SEQ ID NO: 167, amino acids 25-244 of SEQ ID NO: 169, amino acids 25-245 of SEQ ID NO: 171, amino acids 25-244 of SEQ ID NO: 173, amino acids 25-248 of SEQ ID NO: 175, amino acids 25-246 of SEQ ID NO: 177, amino acids 1-249 of SEQ ID NO: 179, amino acids 1-249 of SEQ ID NO: 181, amino acids 1-247 of SEQ ID NO: 183, amino acids 1-251 of SEQ ID NO: 185, amino acids 1-247 of SEQ ID NO: 187, amino acids 25-249 of SEQ ID NO: 189, amino acids 25-248 of SEQ ID NO: 191, amino acids 25-246 of SEQ ID NO: 193, amino acids 25-250 of SEQ ID NO: 195, amino acids 25-245 of SEQ ID NO: 197, amino acids 25-250 of SEQ ID NO: 199, amino acids 25-246 of SEQ ID NO: 201, amino acids 25-243 of SEQ ID NO: 203, amino acids 25-248 of SEQ ID NO: 205, amino acids 25-250 of SEQ ID NO: 207, amino acids 25-249 of SEQ ID NO: 209, amino acids 25-255 of SEQ ID NO: 211, amino acids 25-245 of SEQ ID NO: 213, amino acids 25-244 of SEQ ID NO: 215, amino acids 25-250 of SEQ ID NO: 217, amino acids 25-249 of SEQ ID NO: 219, amino acids 25-247 of SEQ ID NO: 221, amino acids 25-254 of SEQ ID NO: 223, amino acids 25-248 of SEQ ID NO: 225, amino acids 25-249 of SEQ ID NO: 227, amino acids 25-253 of SEQ ID NO: 229, amino acids 25-251 of SEQ ID NO: 231, amino acids 25-244 of SEQ ID NO: 233, amino acids 25-245 of SEQ ID NO: 235, amino acids 25-244 of SEQ ID NO: 237, and amino acids 25-254 of SEQ ID NO: 239.

4. The antibody or antigen-binding fragment of claim 1, comprising:
a heavy chain fragment comprising amino acids 25-251 of SEQ ID NO: 23 and a light chain comprising SEQ ID NO: 241;
a heavy chain fragment comprising amino acids 25-250 of SEQ ID NO: 25 and a light chain comprising SEQ ID NO: 243;
a heavy chain fragment comprising amino acids 25-249 of SEQ ID NO: 27 and a light chain comprising SEQ ID NO: 245;
a heavy chain fragment comprising amino acids 25-252 of SEQ ID NO: 29 and a light chain comprising SEQ ID NO: 247;
a heavy chain fragment comprising amino acids 25-245 of SEQ ID NO: 31 and a light chain comprising SEQ ID NO: 249;
a heavy chain fragment comprising amino acids 25-247 of SEQ ID NO: 33 and a light chain comprising SEQ ID NO: 251;
a heavy chain fragment comprising amino acids 25-254 of SEQ ID NO: 35 and a light chain comprising SEQ ID NO: 253;
a heavy chain fragment comprising amino acids 25-252 of SEQ ID NO: 37 and a light chain comprising SEQ ID NO: 255;
a heavy chain fragment comprising amino acids 25-250 of SEQ ID NO: 39 and a light chain comprising SEQ ID NO: 257;
a heavy chain fragment comprising amino acids 25-249 of SEQ ID NO: 41 and a light chain comprising SEQ ID NO: 259;
a heavy chain fragment comprising amino acids 25-248 of SEQ ID NO: 43 and a light chain comprising SEQ ID NO: 261;
a heavy chain fragment comprising amino acids 25-255 of SEQ ID NO: 45 and a light chain comprising SEQ ID NO: 263;
a heavy chain fragment comprising amino acids 25-245 of SEQ ID NO: 47 and a light chain comprising SEQ ID NO: 265;
a heavy chain fragment comprising amino acids 25-244 of SEQ ID NO: 49 and a light chain comprising SEQ ID NO: 267;
a heavy chain fragment comprising amino acids 25-249 of SEQ ID NO: 51 and a light chain comprising SEQ ID NO: 269;
a heavy chain fragment comprising amino acids 25-256 of SEQ ID NO: 53 and a light chain comprising SEQ ID NO: 271;
a heavy chain fragment comprising amino acids 25-246 of SEQ ID NO: 55 and a light chain comprising SEQ ID NO: 273;
a heavy chain fragment comprising amino acids 25-251 of SEQ ID NO: 57 and a light chain comprising SEQ ID NO: 275;
a heavy chain fragment comprising amino acids 25-245 of SEQ ID NO: 59 and a light chain comprising SEQ ID NO: 277;
a heavy chain fragment comprising amino acids 25-252 of SEQ ID NO: 61 and a light chain comprising SEQ ID NO: 279;
a heavy chain fragment comprising amino acids 25-246 of SEQ ID NO: 63 and a light chain comprising SEQ ID NO: 281;
a heavy chain fragment comprising amino acids 25-249 of SEQ ID NO: 65 and a light chain comprising SEQ ID NO: 283;
a heavy chain fragment comprising amino acids 25-244 of SEQ ID NO: 67 and a light chain comprising SEQ ID NO: 285;
a heavy chain fragment comprising amino acids 25-249 of SEQ ID NO: 69 and a light chain comprising SEQ ID NO: 287;
a heavy chain fragment comprising amino acids 25-244 of SEQ ID NO: 71 and a light chain comprising SEQ ID NO: 289;
a heavy chain fragment comprising amino acids 25-244 of SEQ ID NO: 73 and a light chain comprising SEQ ID NO: 291;
a heavy chain fragment comprising amino acids 25-249 of SEQ ID NO: 75 and a light chain comprising SEQ ID NO: 293;
a heavy chain fragment comprising amino acids 25-247 of SEQ ID NO: 77 and a light chain comprising SEQ ID NO: 295;
a heavy chain fragment comprising amino acids 25-247 of SEQ ID NO: 79 and a light chain comprising SEQ ID NO: 297;
a heavy chain fragment comprising amino acids 25-255 of SEQ ID NO: 81 and a light chain comprising SEQ ID NO: 299;
a heavy chain fragment comprising amino acids 25-246 of SEQ ID NO: 83 and a light chain comprising SEQ ID NO: 301;
a heavy chain fragment comprising amino acids 25-248 of SEQ ID NO: 85 and a light chain comprising SEQ ID NO: 303;
a heavy chain fragment comprising amino acids 25-252 of SEQ ID NO: 87 and a light chain comprising SEQ ID NO: 305;
a heavy chain fragment comprising amino acids 25-248 of SEQ ID NO: 89 and a light chain comprising SEQ ID NO: 307;
a heavy chain fragment comprising amino acids 25-244 of SEQ ID NO: 91 and a light chain comprising SEQ ID NO: 309;
a heavy chain fragment comprising amino acids 25-247 of SEQ ID NO: 93 and a light chain comprising SEQ ID NO: 311;
a heavy chain fragment comprising amino acids 25-254 of SEQ ID NO: 95 and a light chain comprising SEQ ID NO: 313;
a heavy chain fragment comprising amino acids 25-251 of SEQ ID NO: 97 and a light chain comprising SEQ ID NO: 315;
a heavy chain fragment comprising amino acids 25-245 of SEQ ID NO: 99 and a light chain comprising SEQ ID NO: 317;
a heavy chain fragment comprising amino acids 25-257 of SEQ ID NO: 101 and a light chain comprising SEQ ID NO: 319;
a heavy chain fragment comprising amino acids 25-256 of SEQ ID NO: 103 and a light chain comprising SEQ ID NO: 321;
a heavy chain fragment comprising amino acids 25-249 of SEQ ID NO: 105 and a light chain comprising SEQ ID NO: 323;
a heavy chain fragment comprising amino acids 25-248 of SEQ ID NO: 107 and a light chain comprising SEQ ID NO: 325;
a heavy chain fragment comprising amino acids 25-250 of SEQ ID NO: 109 and a light chain comprising SEQ ID NO: 327;
a heavy chain fragment comprising amino acids 25-244 of SEQ ID NO: 111 and a light chain comprising SEQ ID NO: 329;
a heavy chain fragment comprising amino acids 25-247 of SEQ ID NO: 113 and a light chain comprising SEQ ID NO: 331;
a heavy chain fragment comprising amino acids 25-248 of SEQ ID NO: 115 and a light chain comprising SEQ ID NO: 333;
a heavy chain fragment comprising amino acids 25-245 of SEQ ID NO: 117 and a light chain comprising SEQ ID NO: 335;
a heavy chain fragment comprising amino acids 25-253 of SEQ ID NO: 119 and a light chain comprising SEQ ID NO: 337;
a heavy chain fragment comprising amino acids 25-249 of SEQ ID NO: 121 and a light chain comprising SEQ ID NO: 339;
a heavy chain fragment comprising amino acids 25-253 of SEQ ID NO: 123 and a light chain comprising SEQ ID NO: 341
a heavy chain fragment comprising amino acids 25-253 of SEQ ID NO: 125 and a light chain comprising SEQ ID NO: 343;
a heavy chain fragment comprising amino acids 25-251 of SEQ ID NO: 127 and a light chain comprising SEQ ID NO: 345;
a heavy chain fragment comprising amino acids 25-245 of SEQ ID NO: 129 and a light chain comprising SEQ ID NO: 347;
a heavy chain fragment comprising amino acids 25-249 of SEQ ID NO: 131 and a light chain comprising SEQ ID NO: 349;
a heavy chain fragment comprising amino acids 25-250 of SEQ ID NO: 133 and a light chain comprising SEQ ID NO: 351;
a heavy chain fragment comprising amino acids 25-256 of SEQ ID NO: 135 and a light chain comprising SEQ ID NO: 353;
a heavy chain fragment comprising amino acids 25-250 of SEQ ID NO: 137 and a light chain comprising SEQ ID NO: 355;
a heavy chain fragment comprising amino acids 25-247 of SEQ ID NO: 139 and a light chain comprising SEQ ID NO: 357;
a heavy chain fragment comprising amino acids 25-253 of SEQ ID NO: 141 and a light chain comprising SEQ ID NO: 359;
a heavy chain fragment comprising amino acids 25-249 of SEQ ID NO: 143 and a light chain comprising SEQ ID NO: 361;
a heavy chain fragment comprising amino acids 25-246 of SEQ ID NO: 145 and a light chain comprising SEQ ID NO: 363;
a heavy chain fragment comprising amino acids 25-251 of SEQ ID NO: 147 and a light chain comprising SEQ ID NO: 365;
a heavy chain fragment comprising amino acids 25-245 of SEQ ID NO: 149 and a light chain comprising SEQ ID NO: 367;
a heavy chain fragment comprising amino acids 25-244 of SEQ ID NO: 151 and a light chain comprising SEQ ID NO: 369;
a heavy chain fragment comprising amino acids 25-249 of SEQ ID NO: 153 and a light chain comprising SEQ ID NO: 371;
a heavy chain fragment comprising amino acids 25-257 of SEQ ID NO: 155 and a light chain comprising SEQ ID NO: 373;
a heavy chain fragment comprising amino acids 25-250 of SEQ ID NO: 157 and a light chain comprising SEQ ID NO: 375;
a heavy chain fragment comprising amino acids 25-253 of SEQ ID NO: 159 and a light chain comprising SEQ ID NO: 377;
a heavy chain fragment comprising amino acids 25-245 of SEQ ID NO: 161 and a light chain comprising SEQ ID NO: 379;
a heavy chain fragment comprising amino acids 25-260 of SEQ ID NO: 163 and a light chain comprising SEQ ID NO: 381;
a heavy chain fragment comprising amino acids 25-245 of SEQ ID NO: 165 and a light chain comprising SEQ ID NO: 383;
a heavy chain fragment comprising amino acids 25-244 of SEQ ID NO: 167 and a light chain comprising SEQ ID NO: 385;
a heavy chain fragment comprising amino acids 25-244 of SEQ ID NO: 169 and a light chain comprising SEQ ID NO: 387;
a heavy chain fragment comprising amino acids 25-245 of SEQ ID NO: 171 and a light chain comprising SEQ ID NO: 389;
a heavy chain fragment comprising amino acids 25-244 of SEQ ID NO: 173 and a light chain comprising SEQ ID NO: 391;
a heavy chain fragment comprising amino acids 25-248 of SEQ ID NO: 175 and a light chain comprising SEQ ID NO: 393;
a heavy chain fragment comprising amino acids 25-246 of SEQ ID NO: 177 and a light chain comprising SEQ ID NO: 395;
a heavy chain fragment comprising amino acids 25-249 of SEQ ID NO: 179 and a light chain comprising SEQ ID NO: 397;
a heavy chain fragment comprising amino acids 25-249 of SEQ ID NO: 181 and a light chain comprising SEQ ID NO: 399;
a heavy chain fragment comprising amino acids 25-247 of SEQ ID NO: 183 and a light chain comprising SEQ ID NO: 401;
a heavy chain fragment comprising amino acids 25-251 of SEQ ID NO: 185 and a light chain comprising SEQ ID NO: 403;
a heavy chain fragment comprising amino acids 25-247 of SEQ ID NO: 187 and a light chain comprising SEQ ID NO: 405;
a heavy chain fragment comprising amino acids 25-249 of SEQ ID NO: 189 and a light chain comprising SEQ ID NO: 407;
a heavy chain fragment comprising amino acids 25-248 of SEQ ID NO: 191 and a light chain comprising SEQ ID NO: 409;
a heavy chain fragment comprising amino acids 25-246 of SEQ ID NO: 193 and a light chain comprising SEQ ID NO: 411;
a heavy chain fragment comprising amino acids 25-250 of SEQ ID NO: 195 and a light chain comprising SEQ ID NO: 413;
a heavy chain fragment comprising amino acids 25-245 of SEQ ID NO: 197 and a light chain comprising SEQ ID NO: 415;
a heavy chain fragment comprising amino acids 25-250 of SEQ ID NO: 199 and a light chain comprising SEQ ID NO: 417;
a heavy chain fragment comprising amino acids 25-246 of SEQ ID NO: 201 and a light chain comprising SEQ ID NO: 419;
a heavy chain fragment comprising amino acids 25-243 of SEQ ID NO: 203 and a light chain comprising SEQ ID NO: 421;
a heavy chain fragment comprising amino acids 25-248 of SEQ ID NO: 205 and a light chain comprising SEQ ID NO: 423;
a heavy chain fragment comprising amino acids 25-250 of SEQ ID NO: 207 and a light chain comprising SEQ ID NO: 425;
a heavy chain fragment comprising amino acids 25-249 of SEQ ID NO: 209 and a light chain comprising SEQ ID NO: 427;
a heavy chain fragment comprising amino acids 25-255 of SEQ ID NO: 211 and a light chain comprising SEQ ID NO: 429;
a heavy chain fragment comprising amino acids 25-245 of SEQ ID NO: 213 and a light chain comprising SEQ ID NO: 431;
a heavy chain fragment comprising amino acids 25-244 of SEQ ID NO: 215 and a light chain comprising SEQ ID NO: 433;
a heavy chain fragment comprising amino acids 25-250 of SEQ ID NO: 217 and a light chain comprising SEQ ID NO: 435;
a heavy chain fragment comprising amino acids 25-249 of SEQ ID NO: 219 and a light chain comprising SEQ ID NO: 437;
a heavy chain fragment comprising amino acids 25-247 of SEQ ID NO: 221 and a light chain comprising SEQ ID NO: 439;
a heavy chain fragment comprising amino acids 25-254 of SEQ ID NO: 223 and a light chain comprising SEQ ID NO: 441;
a heavy chain fragment comprising amino acids 25-248 of SEQ ID NO: 225 and a light chain comprising SEQ ID NO: 443;
a heavy chain fragment comprising amino acids 25-249 of SEQ ID NO: 227 and a light chain comprising SEQ ID NO: 445;
a heavy chain fragment comprising amino acids 25-253 of SEQ ID NO: 229 and a light chain comprising SEQ ID NO: 447;
a heavy chain fragment comprising amino acids 25-251 of SEQ ID NO: 231 and a light chain comprising SEQ ID NO: 449;
a heavy chain fragment comprising amino acids 25-244 of SEQ ID NO: 233 and a light chain comprising SEQ ID NO: 451;
a heavy chain fragment comprising amino acids 25-245 of SEQ ID NO: 235 and a light chain comprising SEQ ID NO: 453;
a heavy chain fragment comprising amino acids 25-244 of SEQ ID NO: 237 and a light chain comprising SEQ ID NO: 455; or
a heavy chain fragment comprising amino acids 25-254 of SEQ ID NO: 239 and a light chain comprising SEQ ID NO: 457.

5. The antibody or antigen-binding fragment of claim 1, comprising:
(i) a heavy chain amino acid sequence selected from the group consisting of:
SEQ ID NO: 23, SEQ ID NO: 25, SEQ ID NO: 27, SEQ ID NO: 29, SEQ ID NO: 31,
SEQ ID NO: 33, SEQ ID NO: 35, SEQ ID NO: 37, SEQ ID NO: 39, SEQ ID NO: 41,
SEQ ID NO: 43, SEQ ID NO: 45, SEQ ID NO: 47, SEQ ID NO: 49, SEQ ID NO: 51,
SEQ ID NO: 53, SEQ ID NO: 55, SEQ ID NO: 57, SEQ ID NO: 59, SEQ ID NO: 61,
SEQ ID NO: 63, SEQ ID NO: 65, SEQ ID NO: 67, SEQ ID NO: 69, SEQ ID NO: 71,
SEQ ID NO: 73, SEQ ID NO: 75, SEQ ID NO: 77, SEQ ID NO: 79, SEQ ID NO: 81,
SEQ ID NO: 83, SEQ ID NO: 85, SEQ ID NO: 87, SEQ ID NO: 89, SEQ ID NO: 91,
SEQ ID NO: 93, SEQ ID NO: 95, SEQ ID NO: 97, SEQ ID NO: 99, SEQ ID NO: 101,
SEQ ID NO: 103, SEQ ID NO: 105, SEQ ID NO: 107, SEQ ID NO: 109, SEQ ID NO: 111,
SEQ ID NO: 113, SEQ ID NO: 115, SEQ ID NO: 117, SEQ ID NO: 119, SEQ ID NO: 121,
SEQ ID NO: 123, SEQ ID NO: 125, SEQ ID NO: 127, SEQ ID NO: 129, SEQ ID NO: 131,
SEQ ID NO: 133, SEQ ID NO: 135, SEQ ID NO: 137, SEQ ID NO: 139, SEQ ID NO: 141,
SEQ ID NO: 143, SEQ ID NO: 145, SEQ ID NO: 147, SEQ ID NO: 149, SEQ ID NO: 151,
SEQ ID NO: 153, SEQ ID NO: 155, SEQ ID NO: 157, SEQ ID NO: 159, SEQ ID NO: 161,
SEQ ID NO: 163, SEQ ID NO: 165, SEQ ID NO: 167, SEQ ID NO: 169, SEQ ID NO: 171,
SEQ ID NO: 173, SEQ ID NO: 175, SEQ ID NO: 177, SEQ ID NO: 179, SEQ ID NO: 181,
SEQ ID NO: 183, SEQ ID NO: 185, SEQ ID NO: 187, SEQ ID NO: 189, SEQ ID NO: 191,
SEQ ID NO: 193, SEQ ID NO: 195, SEQ ID NO: 197, SEQ ID NO: 199, SEQ ID NO: 201,
SEQ ID NO: 203, SEQ ID NO: 205, SEQ ID NO: 207, SEQ ID NO: 209, SEQ ID NO: 211,
SEQ ID NO: 213, SEQ ID NO: 215, SEQ ID NO: 217, SEQ ID NO: 219, SEQ ID NO: 221,
SEQ ID NO: 223, SEQ ID NO: 225, SEQ ID NO: 227, SEQ ID NO: 229, SEQ ID NO: 231,
SEQ ID NO: 233, SEQ ID NO: 235, SEQ ID NO: 237 and SEQ ID NO: 239; and
(ii) a light chain amino acid sequence selected from the group consisting of:
SEQ ID NO: 241, SEQ ID NO: 243, SEQ ID NO: 245, SEQ ID NO: 247, SEQ ID NO: 249,
SEQ ID NO: 251, SEQ ID NO: 253, SEQ ID NO: 255, SEQ ID NO: 257, SEQ ID NO: 259,
SEQ ID NO: 261, SEQ ID NO: 263, SEQ ID NO: 265, SEQ ID NO: 267, SEQ ID NO: 269,
SEQ ID NO: 271, SEQ ID NO: 273, SEQ ID NO: 275, SEQ ID NO: 277, SEQ ID NO: 279,
SEQ ID NO: 281, SEQ ID NO: 283, SEQ ID NO: 285, SEQ ID NO: 287, SEQ ID NO: 289,
SEQ ID NO: 291, SEQ ID NO: 293, SEQ ID NO: 295, SEQ ID NO: 297, SEQ ID NO: 299,
SEQ ID NO: 301, SEQ ID NO: 303, SEQ ID NO: 305, SEQ ID NO: 307, SEQ ID NO: 309,
SEQ ID NO: 311, SEQ ID NO: 313, SEQ ID NO: 315, SEQ ID NO: 317, SEQ ID NO: 319,
SEQ ID NO: 321, SEQ ID NO: 323, SEQ ID NO: 325, SEQ ID NO: 327, SEQ ID NO: 329,
SEQ ID NO: 331, SEQ ID NO: 333, SEQ ID NO: 335, SEQ ID NO: 337, SEQ ID NO: 339,
SEQ ID NO: 341, SEQ ID NO: 343, SEQ ID NO: 345, SEQ ID NO: 347, SEQ ID NO: 349,
SEQ ID NO: 351, SEQ ID NO: 353, SEQ ID NO: 355, SEQ ID NO: 357, SEQ ID NO: 359,
SEQ ID NO: 361, SEQ ID NO: 363, SEQ ID NO: 365, SEQ ID NO: 367, SEQ ID NO: 369,
SEQ ID NO: 371, SEQ ID NO: 373, SEQ ID NO: 375, SEQ ID NO: 377, SEQ ID NO: 379,
SEQ ID NO: 381, SEQ ID NO: 383, SEQ ID NO: 385, SEQ ID NO: 387, SEQ ID NO: 389,
SEQ ID NO: 391, SEQ ID NO: 393, SEQ ID NO: 395, SEQ ID NO: 397, SEQ ID NO: 399,
SEQ ID NO: 401, SEQ ID NO: 403, SEQ ID NO: 405, SEQ ID NO: 407, SEQ ID NO: 409,
SEQ ID NO: 411, SEQ ID NO: 413, SEQ ID NO: 415, SEQ ID NO: 417, SEQ ID NO: 419,
SEQ ID NO: 421, SEQ ID NO: 423, SEQ ID NO: 425, SEQ ID NO: 427, SEQ ID NO: 429,
SEQ ID NO: 431, SEQ ID NO: 433, SEQ ID NO: 435, SEQ ID NO: 437, SEQ ID NO: 439,
SEQ ID NO: 441, SEQ ID NO: 443, SEQ ID NO: 445, SEQ ID NO: 447, SEQ ID NO: 449,
SEQ ID NO: 451, SEQ ID NO: 453, SEQ ID NO: 455 and SEQ ID NO: 457.

6. An isolated antibody or an antigen-binding fragment thereof that specifically binds ST2, comprising:
(i) a heavy chain variable domain comprising complementarity determining regions (CDRs) as set forth in a heavy chain variable domain amino acid sequence selected from the group consisting of:
amino acids 25-147 of SEQ ID NO: 23, amino acids 25-147 of SEQ ID NO: 25,
amino acids 25-145 of SEQ ID NO: 27, amino acids 25-148 of SEQ ID NO: 29,
amino acids 25-141 of SEQ ID NO: 31, amino acids 25-143 of SEQ ID NO: 33,
amino acids 25-150 of SEQ ID NO: 35, amino acids 25-148 of SEQ ID NO: 37,
amino acids 25-146 of SEQ ID NO: 39, amino acids 25-145 of SEQ ID NO: 41,
amino acids 25-143 of SEQ ID NO: 43, amino acids 25-151 of SEQ ID NO: 45,
amino acids 25-141 of SEQ ID NO: 47, amino acids 25-140 of SEQ ID NO: 49,
amino acids 25-145 of SEQ ID NO: 51, amino acids 25-152 of SEQ ID NO: 53,
amino acids 25-142 of SEQ ID NO: 55, amino acids 25-147 of SEQ ID NO: 57,
amino acids 25-141 of SEQ ID NO: 59, amino acids 25-148 of SEQ ID NO: 61,
amino acids 25-142 of SEQ ID NO: 63, amino acids 25-145 of SEQ ID NO: 65,
amino acids 25-140 of SEQ ID NO: 67, amino acids 25-145 of SEQ ID NO: 69,
amino acids 25-140 of SEQ ID NO: 71, amino acids 25-140 of SEQ ID NO: 73,
amino acids 25-145 of SEQ ID NO: 75, amino acids 25-143 of SEQ ID NO: 77,
amino acids 25-143 of SEQ ID NO: 79, amino acids 25-151 of SEQ ID NO: 81,
amino acids 25-142 of SEQ ID NO: 83, amino acids 25-144 of SEQ ID NO: 85,
amino acids 25-148 of SEQ ID NO: 87, amino acids 25-144 of SEQ ID NO: 89,
amino acids 25-140 of SEQ ID NO: 91, amino acids 25-143 of SEQ ID NO: 93,
amino acids 25-150 of SEQ ID NO: 95, amino acids 25-147 of SEQ ID NO: 97,
amino acids 25-141 of SEQ ID NO: 99, amino acids 25-153 of SEQ ID NO: 101,
amino acids 25-152 of SEQ ID NO: 103, amino acids 25-145 of SEQ ID NO: 105,
amino acids 25-144 of SEQ ID NO: 107, amino acids 25-146 of SEQ ID NO: 109,
amino acids 25-140 of SEQ ID NO: 111, amino acids 25-143 of SEQ ID NO: 113,
amino acids 25-144 of SEQ ID NO: 115, amino acids 25-141 of SEQ ID NO: 117,
amino acids 25-149 of SEQ ID NO: 119, amino acids 25-145 of SEQ ID NO: 121,
amino acids 25-149 of SEQ ID NO: 123, amino acids 25-149 of SEQ ID NO: 125,
amino acids 25-147 of SEQ ID NO: 127, amino acids 25-147 of SEQ ID NO: 129,
amino acids 25-145 of SEQ ID NO: 131, amino acids 25-146 of SEQ ID NO: 133,
amino acids 25-152 of SEQ ID NO: 135, amino acids 25-146 of SEQ ID NO: 137,
amino acids 25-149 of SEQ ID NO: 139, amino acids 25-149 of SEQ ID NO: 141,
amino acids 25-145 of SEQ ID NO: 143, amino acids 25-142 of SEQ ID NO: 145,
amino acids 25-147 of SEQ ID NO: 147, amino acids 25-141 of SEQ ID NO: 149,
amino acids 25-140 of SEQ ID NO: 151, amino acids 25-145 of SEQ ID NO: 153,
amino acids 25-153 of SEQ ID NO: 155, amino acids 25-146 of SEQ ID NO: 157,
amino acids 25-149 of SEQ ID NO: 159, amino acids 25-141 of SEQ ID NO: 161,
amino acids 25-156 of SEQ ID NO: 163, amino acids 25-141 of SEQ ID NO: 165,
amino acids 25-140 of SEQ ID NO: 167, amino acids 25-140 of SEQ ID NO: 169,
amino acids 25-141 of SEQ ID NO: 171, amino acids 25-140 of SEQ ID NO: 173,
amino acids 25-144 of SEQ ID NO: 175, amino acids 25-142 of SEQ ID NO: 177,
amino acids 25-145 of SEQ ID NO: 179, amino acids 25-145 of SEQ ID NO: 181,
amino acids 25-143 of SEQ ID NO: 183, amino acids 25-147 of SEQ ID NO: 185,
amino acids 25-143 of SEQ ID NO: 187, amino acids 25-145 of SEQ ID NO: 189,
amino acids 25-144 of SEQ ID NO: 191, amino acids 25-143 of SEQ ID NO: 193,
amino acids 25-146 of SEQ ID NO: 195, amino acids 25-141 of SEQ ID NO: 197,
amino acids 25-146 of SEQ ID NO: 199, amino acids 25-142 of SEQ ID NO: 201,
amino acids 25-139 of SEQ ID NO: 203, amino acids 25-144 of SEQ ID NO: 205,
amino acids 25-146 of SEQ ID NO: 207, amino acids 25-142 of SEQ ID NO: 209,
amino acids 25-151 of SEQ ID NO: 211, amino acids 25-141 of SEQ ID NO: 213,
amino acids 25-140 of SEQ ID NO: 215, amino acids 25-146 of SEQ ID NO: 217,
amino acids 25-142 of SEQ ID NO: 219, amino acids 25-143 of SEQ ID NO: 221,
amino acids 25-150 of SEQ ID NO: 223, amino acids 25-144 of SEQ ID NO: 225,
amino acids 25-145 of SEQ ID NO: 227, amino acids 25-149 of SEQ ID NO: 229,
amino acids 25-147 of SEQ ID NO: 231, amino acids 25-140 of SEQ ID NO: 233,
amino acids 25-141 of SEQ ID NO: 235, amino acids 25-140 of SEQ ID NO: 237, and
amino acids 25-150 of SEQ ID NO: 239; and
(ii) a light chain variable domain comprising CDRs as set forth in a light chain variable region amino acid sequence selected from the group consisting of:
amino acids 21-132 of SEQ ID NO: 241, amino acids 21-132 of SEQ ID NO: 243,
amino acids 21-128 of SEQ ID NO: 245, amino acids 21-127 of SEQ ID NO: 247,
amino acids 21-127 of SEQ ID NO: 249, amino acids 21-127 of SEQ ID NO: 251,
amino acids 21-131 of SEQ ID NO: 253, amino acids 21-132 of SEQ ID NO: 255,
amino acids 21-127 of SEQ ID NO: 257, amino acids 21-132 of SEQ ID NO: 259,
amino acids 21-127 of SEQ ID NO: 261, amino acids 21-127 of SEQ ID NO: 263,
amino acids 21-132 of SEQ ID NO: 265, amino acids 21-132 of SEQ ID NO: 267,
amino acids 21-127 of SEQ ID NO: 269, amino acids 21-127 of SEQ ID NO: 271,
amino acids 21-127 of SEQ ID NO: 273, amino acids 21-127 of SEQ ID NO: 275,
amino acids 21-127 of SEQ ID NO: 277, amino acids 21-127 of SEQ ID NO: 279,
amino acids 21-127 of SEQ ID NO: 281, amino acids 21-127 of SEQ ID NO: 283,
amino acids 21-127 of SEQ ID NO: 285, amino acids 21-132 of SEQ ID NO: 287,
amino acids 21-127 of SEQ ID NO: 289, amino acids 21-133 of SEQ ID NO: 291,
amino acids 21-127 of SEQ ID NO: 293, amino acids 21-132 of SEQ ID NO: 295,
amino acids 21-127 of SEQ ID NO: 297, amino acids 21-132 of SEQ ID NO: 299,
amino acids 21-127 of SEQ ID NO: 301, amino acids 21-127 of SEQ ID NO: 303,
amino acids 21-132 of SEQ ID NO: 305, amino acids 21-127 of SEQ ID NO: 307,
amino acids 21-127 of SEQ ID NO: 309, amino acids 21-127 of SEQ ID NO: 311,
amino acids 21-127 of SEQ ID NO: 313, amino acids 21-128 of SEQ ID NO: 315,
amino acids 21-132 of SEQ ID NO: 317, amino acids 21-127 of SEQ ID NO: 319,
amino acids 21-133 of SEQ ID NO: 321, amino acids 21-127 of SEQ ID NO: 323,
amino acids 21-127 of SEQ ID NO: 325, amino acids 21-127 of SEQ ID NO: 327,
amino acids 21-127 of SEQ ID NO: 329, amino acids 21-127 of SEQ ID NO: 331,
amino acids 21-127 of SEQ ID NO: 333, amino acids 21-127 of SEQ ID NO: 335,
amino acids 21-127 of SEQ ID NO: 337, amino acids 21-127 of SEQ ID NO: 339,
amino acids 21-128 of SEQ ID NO: 341, amino acids 21-131 of SEQ ID NO: 343,
amino acids 21-127 of SEQ ID NO: 345, amino acids 21-131 of SEQ ID NO: 347,
amino acids 21-132 of SEQ ID NO: 349, amino acids 21-127 of SEQ ID NO: 351,
amino acids 21-127 of SEQ ID NO: 353, amino acids 21-127 of SEQ ID NO: 355,
amino acids 21-127 of SEQ ID NO: 357, amino acids 21-127 of SEQ ID NO: 359,
amino acids 21-132 of SEQ ID NO: 361, amino acids 21-133 of SEQ ID NO: 363,
amino acids 21-132 of SEQ ID NO: 365, amino acids 21-126 of SEQ ID NO: 367,
amino acids 21-127 of SEQ ID NO: 369, amino acids 21-132 of SEQ ID NO: 371,
amino acids 21-127 of SEQ ID NO: 373, amino acids 21-132 of SEQ ID NO: 375,
amino acids 21-132 of SEQ ID NO: 377, amino acids 21-128 of SEQ ID NO: 379,
amino acids 21-127 of SEQ ID NO: 381, amino acids 21-127 of SEQ ID NO: 383,
amino acids 21-127 of SEQ ID NO: 385, amino acids 21-127 of SEQ ID NO: 387,
amino acids 21-127 of SEQ ID NO: 389, amino acids 21-127 of SEQ ID NO: 391,
amino acids 21-133 of SEQ ID NO: 393, amino acids 21-127 of SEQ ID NO: 395,
amino acids 21-127 of SEQ ID NO: 397, amino acids 21-132 of SEQ ID NO: 399,
amino acids 21-127 of SEQ ID NO: 401, amino acids 21-127 of SEQ ID NO: 403,
amino acids 21-127 of SEQ ID NO: 405, amino acids 21-132 of SEQ ID NO: 407,
amino acids 21-127 of SEQ ID NO: 409, amino acids 21-127 of SEQ ID NO: 411,
amino acids 21-127 of SEQ ID NO: 413, amino acids 21-127 of SEQ ID NO: 415,
amino acids 21-127 of SEQ ID NO: 417, amino acids 21-132 of SEQ ID NO: 419,
amino acids 21-127 of SEQ ID NO: 421, amino acids 21-133 of SEQ ID NO: 423,
amino acids 21-132 of SEQ ID NO: 425, amino acids 21-128 of SEQ ID NO: 427,
amino acids 21-132 of SEQ ID NO: 429, amino acids 21-132 of SEQ ID NO: 431,
amino acids 21-127 of SEQ ID NO: 433, amino acids 21-127 of SEQ ID NO: 435,
amino acids 21-127 of SEQ ID NO: 437, amino acids 21-127 of SEQ ID NO: 439,
amino acids 21-126 of SEQ ID NO: 441, amino acids 21-132 of SEQ ID NO: 443,
amino acids 21-127 of SEQ ID NO: 445, amino acids 21-127 of SEQ ID NO: 447,
amino acids 21-127 of SEQ ID NO: 449, amino acids 21-128 of SEQ ID NO: 451,
amino acids 21-127 of SEQ ID NO: 453, amino acids 21-127 of SEQ ID NO: 455 and
amino acids 21-133 of SEQ ID NO: 457.

7. A recombinant antibody or an antigen-binding fragment thereof that specifically binds ST2, wherein the antibody, or antigen-binding fragment thereof, comprises six complementarity determining regions (CDRs): CDRH1, CDRH2, CDRH3, CDRL1, CDRL2, and CDRL3,
wherein CDRH1 comprises an amino acid sequence that has at least 85% sequence identity to a CDRH1 amino acid sequence selected from the group consisting of:
SEQ ID NO: 458, SEQ ID NO: 464, SEQ ID NO: 470, SEQ ID NO: 476, SEQ ID NO: 482,
SEQ ID NO: 488, SEQ ID NO: 494, SEQ ID NO: 500, SEQ ID NO: 506, SEQ ID NO: 512,
SEQ ID NO: 518, SEQ ID NO: 524, SEQ ID NO: 530, SEQ ID NO: 536, SEQ ID NO: 542,
SEQ ID NO: 548, SEQ ID NO: 554, SEQ ID NO: 560, SEQ ID NO: 566, SEQ ID NO: 572,
SEQ ID NO: 578, SEQ ID NO: 584, SEQ ID NO: 590, SEQ ID NO: 596, SEQ ID NO: 602,
SEQ ID NO: 608, SEQ ID NO: 614, SEQ ID NO: 620, SEQ ID NO: 626, SEQ ID NO: 632,
SEQ ID NO: 638, SEQ ID NO: 644, SEQ ID NO: 650, SEQ ID NO: 656, SEQ ID NO: 662,
SEQ ID NO: 668, SEQ ID NO: 674, SEQ ID NO: 680, SEQ ID NO: 686, SEQ ID NO: 692,
SEQ ID NO: 698, SEQ ID NO: 704, SEQ ID NO: 710, SEQ ID NO: 716, SEQ ID NO: 722,
SEQ ID NO: 728, SEQ ID NO: 734, SEQ ID NO: 740, SEQ ID NO: 746, SEQ ID NO: 752,
SEQ ID NO: 758, SEQ ID NO: 764, SEQ ID NO: 770, SEQ ID NO: 776, SEQ ID NO: 782,
SEQ ID NO: 788, SEQ ID NO: 794, SEQ ID NO: 800, SEQ ID NO: 806, SEQ ID NO: 812,
SEQ ID NO: 818, SEQ ID NO: 824, SEQ ID NO: 830, SEQ ID NO: 836, SEQ ID NO: 842,
SEQ ID NO: 848, SEQ ID NO: 854, SEQ ID NO: 860, SEQ ID NO: 866, SEQ ID NO: 872,
SEQ ID NO: 878, SEQ ID NO: 884, SEQ ID NO: 890, SEQ ID NO: 896, SEQ ID NO: 902,
SEQ ID NO: 908, SEQ ID NO: 914, SEQ ID NO: 920, SEQ ID NO: 926, SEQ ID NO: 932,
SEQ ID NO: 938, SEQ ID NO: 944, SEQ ID NO: 950, SEQ ID NO: 956, SEQ ID NO: 962,
SEQ ID NO: 968, SEQ ID NO: 974, SEQ ID NO: 980, SEQ ID NO: 986, SEQ ID NO: 992,
SEQ ID NO: 998, SEQ ID NO: 1004, SEQ ID NO: 1010, SEQ ID NO: 1016, SEQ ID NO: 1022,
SEQ ID NO: 1028, SEQ ID NO: 1034, SEQ ID NO: 1040, SEQ ID NO: 1046, SEQ ID NO: 1052,
SEQ ID NO: 1058, SEQ ID NO: 1064, SEQ ID NO: 1070, SEQ ID NO: 1076, SEQ ID NO: 1082,
SEQ ID NO: 1088, SEQ ID NO: 1094, SEQ ID NO: 1100 and SEQ ID NO: 1106;
wherein CDRH2 comprises an amino acid sequence that has at least 85% sequence identity to a CDRH2 amino acid sequence selected from the group consisting of:
SEQ ID NO: 459, SEQ ID NO: 465, SEQ ID NO: 471, SEQ ID NO: 477, SEQ ID NO: 483,
SEQ ID NO: 489, SEQ ID NO: 495, SEQ ID NO: 501, SEQ ID NO: 507, SEQ ID NO: 513,
SEQ ID NO: 519, SEQ ID NO: 525, SEQ ID NO: 531, SEQ ID NO: 537, SEQ ID NO: 543,
SEQ ID NO: 549, SEQ ID NO: 555, SEQ ID NO: 561, SEQ ID NO: 567, SEQ ID NO: 573,
SEQ ID NO: 579, SEQ ID NO: 585, SEQ ID NO: 591, SEQ ID NO: 597, SEQ ID NO: 603,
SEQ ID NO: 609, SEQ ID NO: 615, SEQ ID NO: 621, SEQ ID NO: 627, SEQ ID NO: 633,
SEQ ID NO: 639, SEQ ID NO: 645, SEQ ID NO: 651, SEQ ID NO: 657, SEQ ID NO: 663,
SEQ ID NO: 669, SEQ ID NO: 675, SEQ ID NO: 681, SEQ ID NO: 687, SEQ ID NO: 693,
SEQ ID NO: 699, SEQ ID NO: 705, SEQ ID NO: 711, SEQ ID NO: 717, SEQ ID NO: 723,
SEQ ID NO: 729, SEQ ID NO: 735, SEQ ID NO: 741, SEQ ID NO: 747, SEQ ID NO: 753,
SEQ ID NO: 759, SEQ ID NO: 765, SEQ ID NO: 771, SEQ ID NO: 777, SEQ ID NO: 783,
SEQ ID NO: 789, SEQ ID NO: 795, SEQ ID NO: 801, SEQ ID NO: 807, SEQ ID NO: 813,
SEQ ID NO: 819, SEQ ID NO: 825, SEQ ID NO: 831, SEQ ID NO: 837, SEQ ID NO: 843,
SEQ ID NO: 849, SEQ ID NO: 855, SEQ ID NO: 861, SEQ ID NO: 867, SEQ ID NO: 873,
SEQ ID NO: 879, SEQ ID NO: 885, SEQ ID NO: 891, SEQ ID NO: 897, SEQ ID NO: 903,
SEQ ID NO: 909, SEQ ID NO: 915, SEQ ID NO: 921, SEQ ID NO: 927, SEQ ID NO: 933,
SEQ ID NO: 939, SEQ ID NO: 945, SEQ ID NO: 951, SEQ ID NO: 957, SEQ ID NO: 963,
SEQ ID NO: 969, SEQ ID NO: 975, SEQ ID NO: 981, SEQ ID NO: 987, SEQ ID NO: 993,
SEQ ID NO: 999, SEQ ID NO: 1005, SEQ ID NO: 1011, SEQ ID NO: 1017, SEQ ID NO: 1023,
SEQ ID NO: 1029, SEQ ID NO: 1035, SEQ ID NO: 1041, SEQ ID NO: 1047, SEQ ID NO: 1053,
SEQ ID NO: 1059, SEQ ID NO: 1065, SEQ ID NO: 1071, SEQ ID NO: 1077, SEQ ID NO: 1083,
SEQ ID NO: 1089, SEQ ID NO: 1095, SEQ ID NO: 1101 and SEQ ID NO: 1107,
wherein CDRH3 comprises an amino acid sequence that has at least 85% sequence identity to a CDRH3 amino acid sequence selected from the group consisting of:
SEQ ID NO: 460, SEQ ID NO: 466, SEQ ID NO: 472, SEQ ID NO: 478, SEQ ID NO: 484,
SEQ ID NO: 490, SEQ ID NO: 496, SEQ ID NO: 502, SEQ ID NO: 508, SEQ ID NO: 514,
SEQ ID NO: 520, SEQ ID NO: 526, SEQ ID NO: 532, SEQ ID NO: 538, SEQ ID NO: 544,
SEQ ID NO: 550, SEQ ID NO: 556, SEQ ID NO: 562, SEQ ID NO: 568, SEQ ID NO: 574,
SEQ ID NO: 580, SEQ ID NO: 586, SEQ ID NO: 592, SEQ ID NO: 598, SEQ ID NO: 604,
SEQ ID NO: 610, SEQ ID NO: 616, SEQ ID NO: 622, SEQ ID NO: 628, SEQ ID NO: 634,
SEQ ID NO: 640, SEQ ID NO: 646, SEQ ID NO: 652, SEQ ID NO: 658, SEQ ID NO: 664,
SEQ ID NO: 670, SEQ ID NO: 676, SEQ ID NO: 682, SEQ ID NO: 688, SEQ ID NO: 694,
SEQ ID NO: 700, SEQ ID NO: 706, SEQ ID NO: 712, SEQ ID NO: 718, SEQ ID NO: 724,
SEQ ID NO: 730, SEQ ID NO: 736, SEQ ID NO: 742, SEQ ID NO: 748, SEQ ID NO: 754,
SEQ ID NO: 760, SEQ ID NO: 766, SEQ ID NO: 772, SEQ ID NO: 778, SEQ ID NO: 784,
SEQ ID NO: 790, SEQ ID NO: 796, SEQ ID NO: 802, SEQ ID NO: 808, SEQ ID NO: 814,
SEQ ID NO: 820, SEQ ID NO: 826, SEQ ID NO: 832, SEQ ID NO: 838, SEQ ID NO: 844,
SEQ ID NO: 850, SEQ ID NO: 856, SEQ ID NO: 862, SEQ ID NO: 868, SEQ ID NO: 874,
SEQ ID NO: 880, SEQ ID NO: 886, SEQ ID NO: 892, SEQ ID NO: 898, SEQ ID NO: 904,
SEQ ID NO: 910, SEQ ID NO: 916, SEQ ID NO: 922, SEQ ID NO: 928, SEQ ID NO: 934,
SEQ ID NO: 940, SEQ ID NO: 946, SEQ ID NO: 952, SEQ ID NO: 958, SEQ ID NO: 964,
SEQ ID NO: 970, SEQ ID NO: 976, SEQ ID NO: 982, SEQ ID NO: 988, SEQ ID NO: 994,
SEQ ID NO: 1000, SEQ ID NO: 1006, SEQ ID NO: 1012, SEQ ID NO: 1018, SEQ ID NO: 1024,
SEQ ID NO: 1030, SEQ ID NO: 1036, SEQ ID NO: 1042, SEQ ID NO: 1048, SEQ ID NO: 1054,
SEQ ID NO: 1060, SEQ ID NO: 1066, SEQ ID NO: 1072, SEQ ID NO: 1078, SEQ ID NO: 1084,
SEQ ID NO: 1090, SEQ ID NO: 1096, SEQ ID NO: 1102 and SEQ ID NO: 1108,
wherein CDRL1 comprises an amino acid sequence that has at least 85% sequence identity to a CDRL1 amino acid sequence selected from the group consisting of:
SEQ ID NO: 461, SEQ ID NO: 467, SEQ ID NO: 473, SEQ ID NO: 479, SEQ ID NO: 485,
SEQ ID NO: 491, SEQ ID NO: 497, SEQ ID NO: 503, SEQ ID NO: 509, SEQ ID NO: 515,
SEQ ID NO: 521, SEQ ID NO: 527, SEQ ID NO: 533, SEQ ID NO: 539, SEQ ID NO: 545,
SEQ ID NO: 551, SEQ ID NO: 557, SEQ ID NO: 563, SEQ ID NO: 569, SEQ ID NO: 575,
SEQ ID NO: 581, SEQ ID NO: 587, SEQ ID NO: 593, SEQ ID NO: 599, SEQ ID NO: 605,
SEQ ID NO: 611, SEQ ID NO: 617, SEQ ID NO: 623, SEQ ID NO: 629, SEQ ID NO: 635,
SEQ ID NO: 641, SEQ ID NO: 647, SEQ ID NO: 653, SEQ ID NO: 659, SEQ ID NO: 665,
SEQ ID NO: 671, SEQ ID NO: 677, SEQ ID NO: 683, SEQ ID NO: 689, SEQ ID NO: 695,
SEQ ID NO: 701, SEQ ID NO: 707, SEQ ID NO: 713, SEQ ID NO: 719, SEQ ID NO: 725,
SEQ ID NO: 731, SEQ ID NO: 737, SEQ ID NO: 743, SEQ ID NO: 749, SEQ ID NO: 755,
SEQ ID NO: 761, SEQ ID NO: 767, SEQ ID NO: 773, SEQ ID NO: 779, SEQ ID NO: 785,
SEQ ID NO: 791, SEQ ID NO: 797, SEQ ID NO: 803, SEQ ID NO: 809, SEQ ID NO: 815,
SEQ ID NO: 821, SEQ ID NO: 827, SEQ ID NO: 833, SEQ ID NO: 839, SEQ ID NO: 845,
SEQ ID NO: 851, SEQ ID NO: 857, SEQ ID NO: 863, SEQ ID NO: 869, SEQ ID NO: 875,
SEQ ID NO: 881, SEQ ID NO: 887, SEQ ID NO: 893, SEQ ID NO: 899, SEQ ID NO: 905,
SEQ ID NO: 911, SEQ ID NO: 917, SEQ ID NO: 923, SEQ ID NO: 929, SEQ ID NO: 935,
SEQ ID NO: 941, SEQ ID NO: 947, SEQ ID NO: 953, SEQ ID NO: 959, SEQ ID NO: 965,
SEQ ID NO: 971, SEQ ID NO: 977, SEQ ID NO: 983, SEQ ID NO: 989, SEQ ID NO: 995,
SEQ ID NO: 1001, SEQ ID NO: 1007, SEQ ID NO: 1013, SEQ ID NO: 1019, SEQ ID NO: 1025,
SEQ ID NO: 1031, SEQ ID NO: 1037, SEQ ID NO: 1043, SEQ ID NO: 1049, SEQ ID NO: 1055,
SEQ ID NO: 1061, SEQ ID NO: 1067, SEQ ID NO: 1073, SEQ ID NO: 1079, SEQ ID NO: 1085,
SEQ ID NO: 1091, SEQ ID NO: 1097, SEQ ID NO: 1103 and SEQ ID NO: 1109,
wherein CDRL2 comprises an amino acid sequence that that has at least 85% sequence identity to a CDRL2 sequence selected from the group consisting of:
SEQ ID NO: 462, SEQ ID NO: 468, SEQ ID NO: 474, SEQ ID NO: 480, SEQ ID NO: 486,
SEQ ID NO: 492, SEQ ID NO: 498, SEQ ID NO: 504, SEQ ID NO: 510, SEQ ID NO: 516,
SEQ ID NO: 522, SEQ ID NO: 528, SEQ ID NO: 534, SEQ ID NO: 540, SEQ ID NO: 546,
SEQ ID NO: 552, SEQ ID NO: 558, SEQ ID NO: 564, SEQ ID NO: 570, SEQ ID NO: 576,
SEQ ID NO: 582, SEQ ID NO: 588, SEQ ID NO: 594, SEQ ID NO: 600, SEQ ID NO: 606,
SEQ ID NO: 612, SEQ ID NO: 618, SEQ ID NO: 624, SEQ ID NO: 630, SEQ ID NO: 636,
SEQ ID NO: 642, SEQ ID NO: 648, SEQ ID NO: 654, SEQ ID NO: 660, SEQ ID NO: 666,
SEQ ID NO: 672, SEQ ID NO: 678, SEQ ID NO: 684, SEQ ID NO: 690, SEQ ID NO: 696,
SEQ ID NO: 702, SEQ ID NO: 708, SEQ ID NO: 714, SEQ ID NO: 720, SEQ ID NO: 726,
SEQ ID NO: 732, SEQ ID NO: 738, SEQ ID NO: 744, SEQ ID NO: 750, SEQ ID NO: 756,
SEQ ID NO: 762, SEQ ID NO: 768, SEQ ID NO: 774, SEQ ID NO: 780, SEQ ID NO: 786,
SEQ ID NO: 792, SEQ ID NO: 798, SEQ ID NO: 804, SEQ ID NO: 810, SEQ ID NO: 816,
SEQ ID NO: 822, SEQ ID NO: 828, SEQ ID NO: 834, SEQ ID NO: 840, SEQ ID NO: 846,
SEQ ID NO: 852, SEQ ID NO: 858, SEQ ID NO: 864, SEQ ID NO: 870, SEQ ID NO: 876,
SEQ ID NO: 882, SEQ ID NO: 888, SEQ ID NO: 894, SEQ ID NO: 900, SEQ ID NO: 906,
SEQ ID NO: 912, SEQ ID NO: 918, SEQ ID NO: 924, SEQ ID NO: 930, SEQ ID NO: 936,
SEQ ID NO: 942, SEQ ID NO: 948, SEQ ID NO: 954, SEQ ID NO: 960, SEQ ID NO: 966,
SEQ ID NO: 972, SEQ ID NO: 978, SEQ ID NO: 984, SEQ ID NO: 990, SEQ ID NO: 996,
SEQ ID NO: 1002, SEQ ID NO: 1008, SEQ ID NO: 1014, SEQ ID NO: 1020, SEQ ID NO: 1026, SEQ ID NO: 1032, SEQ ID NO: 1038, SEQ ID NO: 1044, SEQ ID NO: 1050, SEQ ID NO: 1056, SEQ ID NO: 1062, SEQ ID NO: 1068, SEQ ID NO: 1074, SEQ ID NO: 1080, SEQ ID NO: 1086, SEQ ID NO: 1092, SEQ ID NO: 1098, SEQ ID NO: 1104 and SEQ ID NO: 1110,
wherein CDRL3 comprises an amino acid sequence that has at least 85% sequence identity to a CDRL3 sequence selected from the group consisting of:
SEQ ID NO: 463, SEQ ID NO: 469, SEQ ID NO: 475, SEQ ID NO: 481, SEQ ID NO: 487,
SEQ ID NO: 493, SEQ ID NO: 499, SEQ ID NO: 505, SEQ ID NO: 511, SEQ ID NO: 517,
SEQ ID NO: 523, SEQ ID NO: 529, SEQ ID NO: 535, SEQ ID NO: 541, SEQ ID NO: 547,
SEQ ID NO: 553, SEQ ID NO: 559, SEQ ID NO: 565, SEQ ID NO: 571, SEQ ID NO: 577,
SEQ ID NO: 583, SEQ ID NO: 589, SEQ ID NO: 595, SEQ ID NO: 601, SEQ ID NO: 607,
SEQ ID NO: 613, SEQ ID NO: 619, SEQ ID NO: 625, SEQ ID NO: 631, SEQ ID NO: 637,
SEQ ID NO: 643, SEQ ID NO: 649, SEQ ID NO: 655, SEQ ID NO: 661, SEQ ID NO: 667,
SEQ ID NO: 673, SEQ ID NO: 679, SEQ ID NO: 685, SEQ ID NO: 691, SEQ ID NO: 697,
SEQ ID NO: 703, SEQ ID NO: 709, SEQ ID NO: 715, SEQ ID NO: 721, SEQ ID NO: 727,
SEQ ID NO: 733, SEQ ID NO: 739, SEQ ID NO: 745, SEQ ID NO: 751, SEQ ID NO: 757,
SEQ ID NO: 763, SEQ ID NO: 769, SEQ ID NO: 775, SEQ ID NO: 781, SEQ ID NO: 787,
SEQ ID NO: 793, SEQ ID NO: 799, SEQ ID NO: 805, SEQ ID NO: 811, SEQ ID NO: 817,
SEQ ID NO: 823, SEQ ID NO: 829, SEQ ID NO: 835, SEQ ID NO: 841, SEQ ID NO: 847,
SEQ ID NO: 853, SEQ ID NO: 859, SEQ ID NO: 865, SEQ ID NO: 871, SEQ ID NO: 877,
SEQ ID NO: 883, SEQ ID NO: 889, SEQ ID NO: 895, SEQ ID NO: 901, SEQ ID NO: 907,
SEQ ID NO: 913, SEQ ID NO: 919, SEQ ID NO: 925, SEQ ID NO: 931, SEQ ID NO: 937,
SEQ ID NO: 943, SEQ ID NO: 949, SEQ ID NO: 955, SEQ ID NO: 961, SEQ ID NO: 967,
SEQ ID NO: 973, SEQ ID NO: 979, SEQ ID NO: 985, SEQ ID NO: 991, SEQ ID NO: 997,
SEQ ID NO: 1003, SEQ ID NO: 1009, SEQ ID NO: 1015, SEQ ID NO: 1021, SEQ ID NO: 1027,
SEQ ID NO: 1033, SEQ ID NO: 1039, SEQ ID NO: 1045, SEQ ID NO: 1051, SEQ ID NO: 1057,
SEQ ID NO: 1063, SEQ ID NO: 1069, SEQ ID NO: 1075, SEQ ID NO: 1081, SEQ ID NO: 1087,
SEQ ID NO: 1093, SEQ ID NO: 1099, SEQ ID NO: 1105 and SEQ ID NO: 1111.

8. The recombinant antibody, or the antigen-binding fragment thereof of claim 1, which is a fully human antibody.

9. The antigen-binding fragment of claim 1, wherein the fragment is selected from the group consisting of a Fab fragment, a F(ab′)2 fragment, a Fd fragment, a Fv fragment, a dAb fragment, a single chain Fv (scFv), a dimerized variable region (V region) fragment (diabody), and a disulfide-stabilized V region fragment (dsFv).

10. A fusion protein comprising:
a) a human IL-2 protein domain;
b) an immunoglobulin Fc protein domain; and
c) an ST2-binding domain comprising the antibody specific for ST2, or the antigen-binding fragment thereof of claim 1.

11. The fusion protein of claim 10, further comprising at least one peptide linker domain.

12. The fusion protein of claim 10, wherein the human IL-2 protein domain comprises human IL-2 with a substitution selected from the group consisting of: T3A, N88R, N88G, D20H, C125S, Q126L, and Q126F.

13. The fusion protein of claim 10, wherein the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9.

14. The fusion protein of claim 10, wherein the peptide linker domain comprises the amino acid sequence of SEQ ID NO: 6.

15. The fusion protein of claim 10, comprising a first peptide linker domain and a second peptide linker domain.

16. The fusion protein of claim 15, wherein each domain has an amino-terminus (N-terminus) and a carboxy terminus (C-terminus); and wherein the fusion protein is configured so that
a) the C-terminus of the human IL-2 protein domain is fused through a peptide bond to the N-terminus of the first peptide linker domain;
b) the N-terminus of the IgG Fc protein domain is fused through a peptide bond to the C-terminus of the first peptide linker domain;
c) the N-terminus of the second peptide linker domain is fused through a peptide bond to the C-terminus of the IgG Fc protein domain; and
d) the N-terminus of the ST2-binding domain is fused through a peptide bond to the C-terminus of the second peptide linker domain.

17. A nucleic acid encoding the fusion protein of claim 10.

18. A dimeric protein comprising the fusion protein of claim 10.

19. A dimeric protein comprising a first fusion protein and a second fusion protein, wherein:
a. each fusion protein comprises an immunoglobulin (IgG) Fc protein domain and at least one additional protein domain selected from the group consisting of
i. a human IL-2 protein domain; and
ii. an ST2-binding domain comprising the antibody or antigen-binding fragment of claim 1; and
b. the dimeric protein comprises at least one of the human IL-2 protein domain and at least one of the ST2-binding domain.

20. The dimeric protein of claim 19, wherein
a. the first fusion protein comprises the human IL-2 protein domain, a first immunoglobulin Fc protein domain, and a first peptide linker; and
b. the second fusion protein comprises the ST2-binding domain, a second immunoglobulin Fc protein domain, and a second peptide linker domain.

21. The dimeric protein of claim 19, wherein
a. each domain has an amino-terminus (N-terminus) and a carboxy terminus (C-terminus);
b. the first fusion protein is configured so that
i. the C-terminus of the human IL-2 protein domain is fused through a peptide bond to the N-terminus of the first peptide linker domain; and
ii. the N-terminus of the first IgG Fc protein domain is fused through a peptide bond to the C-terminus of the first peptide linker domain; and
c. the second fusion protein is configured so that
i. the C-terminus of the second IgG Fc protein domain is fused through a peptide bond to the N-terminus of the second peptide linker domain; and
ii. the N-terminus of the ST2-binding domain is fused through a peptide bond to the C-terminus of the second peptide linker domain.

22. The dimeric protein of claim 19, wherein at least one of the fusion proteins further comprises at least one peptide linker domain.

23. The dimeric protein of claim 19, wherein the peptide linker domain comprises the amino acid sequence of SEQ ID NO: 6.

24. The dimeric protein of claim 19, wherein the human IL-2 protein domain comprises human IL-2 with a substitution selected from the group consisting of: T3A, N88R, N88G, D20H, C125S, Q126L, and Q126F, relative to the amino acid sequence of SEQ ID NO: 2.

25. The dimeric protein of claim 19, wherein the immunoglobulin Fc protein domain comprises an amino acid sequence selected from the group consisting of the human IgG1 Fc variant of SEQ ID NO: 4, SEQ ID NO: 5, SEQ ID NO: 7, SEQ ID NO: 8 or SEQ ID NO: 9.

26. A pharmaceutical composition comprising the dimeric protein of claim 19.

27. A method for treating a condition, the method comprising administering to a subject in need thereof a therapeutically-effective amount of the pharmaceutical composition of claim 26.

28. The method of claim 27, wherein the condition is selected from the group consisting of an inflammatory myopathy, muscular dystrophy, polymyositis, dermatomyositis, an inflammatory condition of adipose tissue, an inflammatory condition of the colon, an inflammatory condition of the lung, Graft-vs-Host Disease, Pemphigus Vulgaris, Systemic Lupus Erythematosus, Scleroderma, Ulcerative Colitis, Crohn's Disease, Psoriasis, Type 1 Diabetes, Multiple Sclerosis, Amyotrophic Lateral Sclerosis, Alopecia Areata, Uveitis, Neuromyelitis Optica, and Duchenne Muscular Dystrophy.

29. The method of claim 27, wherein the administration is subcutaneous.

30. A method of selectively activating an ST2⁺ regulatory T cell relative to an ST2⁻ regulatory T cell in a subject, the method comprising administering to the subject a therapeutically effective amount of the pharmaceutical composition of claim 26.
